(19) United States
(12) Patent Application Publication
(10) Pub. No.: US 2003/0073634 A1
(43) Pub. Date: Apr. 17, 2003

(54) Methods of treating obesity

(76) Inventor: Martin Myers, Jamaica Plain, MA (US)

Correspondence Address: FISH & RICHARDSON PC, 225 FRANKLIN ST, BOSTON, MA, 02110, US

(21) Appl. No.: 10/226,579

(22) Filed: Aug. 23, 2002

Related U.S. Application Data
(60) Provisional application No. 60/314,976, filed on Aug. 24, 2001.

(51) Int. Cl. A61K 38/17

(52) U.S. Cl. 514/12

(57) ABSTRACT

The invention provides methods of modulating appetite, body weight and/or reproductive function in a mammal by modulating leptin receptor long form (LRb)-STAT3 signaling.

### CROSS REFERENCE TO RELATED APPLICATIONS

This application claims the benefit of provisional application No. 60/314,976, filed Aug. 24, 2001, the contents of which are incorporated herein by reference in their entirety.

### BACKGROUND OF THE INVENTION

Leptin secretion by adipocytes communicates the status of body energy stores to the central nervous system by activating the leptin receptor long form (LRb), thereby regulating food intake, metabolic rate, and endocrine function. Disruption of the entire LRb in db/db mice results in obesity and endocrine dysfunction, e.g., infertility and shortness (Chua et al., 1996, Science 271, 994-996; Lee et al., 1996, Nature 379, 632-635; Chen et al., 1996, Cell 84, 491-495; Tartaglia et al., 1995, Cell 83, 1263-1271). The LRb form of the leptin receptor has a cytoplasmic domain of 302 amino acids that includes several motifs for protein-protein interaction. Leptin binding to LRb activates Jak2, leading to the phosphorylation of Tyr⁹⁸⁵ and Tyr¹¹³⁸, residues unique to LRb. Tyr⁹⁸⁵ recruits the binding of SHP-2 and SOCS3 and controls the activation of ERK, while Tyr¹¹³⁸ activates STAT3 (Bjorbaek et al., 1997, J. Biol. Chem. 272, 32686-32695 (1997); Banks et al., 2000, J Biol Chem 275, 14563-14572; Stahl et al., 1995, Science 267, 1349-1353); White et al., 1997, J. Biol. Chem. 272, 4065-4071).

Stat3 is a transcription factor that acts as a key signaling molecule for many cytokines and growth-factor receptors (Akira, 2000, Oncogene 19(21):2607-11). Stat3 is activated by tyrosine phosphorylation at Tyr705, which induces dimerization, nuclear translocation and DNA binding. Transcriptional activation appears to be activated by serine phosphorylation at Ser727.

### SUMMARY OF THE INVENTION

The invention is based, in part, on the discovery that certain signaling pathways of the leptin receptor, e.g., the LRb-STAT3 pathway, play a role in a subset of leptin functions (e.g., control of body weight) whereas other leptin receptor signaling pathways play a role in other leptin functions (e.g., fertility). It was found that when the leptin receptor gene (lep-r) has been replaced with an allele containing a substitution of LRb Tyr¹¹³⁸ to Ser1138 (leprS1138), the LRb-STAT3 signal is specifically disrupted. Similar to db/db mice, animals homozygous for leprS1138 (s/s) are obese, hyperglycemic, and have elevated glucocorticoid levels. In marked contrast to db/db mice, it was unexpectedly found that the s/s mice described herein are fertile and of normal length. Furthermore, while both the hypothalamic melanocortin and neuropeptide Y (NPY) systems are dysregulated in db/db mice, melanocortin levels are altered in s/s mice, but NPY levels are normal. While not wanting to be bound by theory, it is believed that the LRb-STAT3 pathway controls a subset of leptin functions involved specifically in control of appetite and body weight. In particular, the LRb-STAT3 pathway is required to mediate satiety and body energy homeostasis via the melanocortin system (and thereby to regulate body weight), but is not required for control of fertility, height, normal control of NPY function and the hypothalamic-gonadal axis.

Accordingly, in one aspect, the present invention features a method of modulating body weight and/or appetite and/or caloric intake in a mammal. The method includes modulating LRb-STAT3 signaling to thereby modulate body weight, appetite and/or caloric intake. Preferably, body weight, appetite and/or caloric intake is modulated without an effect on a reproductive function, e.g., fertility. In one embodiment, the method includes: optionally, identifying a mammal in need having its body weight modulated, but preferably without effecting fertility or height; and administering to the mammal an agent that modulates leptin receptor long form (LRb)-STAT3 signaling. In a preferred embodiment, the method includes evaluating a reproductive function of the mammal, e.g., evaluating fertility, e.g., evaluating sperm or egg production or viability, evaluating the timing or function of the female reproductive cycle, evaluating gonad histology.

The mammal can be a primate, e.g., an ape or a human; a rodent, e.g., a rat, mouse, or an animal model for a leptin related disorder, e.g., a ob/ob mouse, a db/db mouse, a Zucker rat, or a nod mouse.

In one aspect, the agent promotes or increases LRb-STAT3 signaling, to thereby decrease appetite and/or body weight and/or caloric intake. In a preferred embodiment, the mammal can be overweight, or obese and/or the mammal can have a compulsive eating behavior or other eating disorder. Preferably, appetite, body weight and/or caloric intake are decreased without an effect on a reproductive function, e.g., fertility. In a preferred embodiment, LRb-STAT3 signaling is increased by administering an agent that promotes an LRb-STAT3 signaling activity. An agent that promotes an LRb-STAT3 signaling activity can be one or more of: an agent that increases or mimics LRb Tyr¹¹³⁸ (or a corresponding tyrosine, e.g., Tyr¹¹⁴¹ of human LRb) phosphorylation; an agent that increases STAT3 levels and/or activity; an agent that increases proopiomelanocortin (POMC) levels and/or activity; an agent that increases αMSH levels and/or activity; an agent that increases MC-3 and/or MC-4 levels and/or activity.

In a preferred embodiment, the agent increases or mimics the phosphorylation of the tyrosine located at about amino acid 1138 of LRb (e.g., mouse LRb Tyr¹¹³⁸ or the corresponding tyrosine residue in another mammal, e.g., human LRb Tyr¹¹⁴¹). The agent can be, e.g., a kinase that phosphorylates Tyr¹¹³⁸ or a corresponding tyrosine of LRb (e.g., Jak2 or a functional fragment or analog thereof); an analog of LRb that mimics phosphorylated LRb, e.g., an LRb analog in which Tyr¹¹³⁸ or a corresponding tyrosine is substituted with Asp or Glu; a kinase agonist, e.g., a Jak2 agonist that increases Jak2 kinase activity.

In a preferred embodiment, the agent increases STAT3 activity. The agent can be, e.g., a STAT3 polypeptide or a functional fragment or analog thereof, preferably a transcriptionally active STAT3 polypeptide or a functional fragment or analog thereof; a peptide or protein agonist of STAT3 that increases the activity, e.g., the transcriptional activity, of STAT3 (e.g., by increasing phosphorylation of STAT3 (e.g., at Tyr705 or Ser727), by promoting or stabilizing dimerization of STAT3, by increasing nuclear translocation of STAT3, by promoting or stabilizing STAT3 binding to DNA); a small molecule that increases expression of STAT3, e.g., by binding to the promoter region of the STAT3 gene; an antibody, e.g., an antibody that binds to and stabilizes or assists the binding of STAT3 to a STAT3 binding partner (e.g., LRb, another STAT3 molecule, or DNA); or a nucleotide sequence encoding a STAT3 polypeptide or functional fragment or analog thereof. The nucleotide sequence can be a genomic sequence or a cDNA sequence. The nucleotide sequence can include: a STAT3 coding region; a promoter sequence, e.g., a promoter sequence from a STAT3 gene or from another gene; an enhancer sequence; untranslated regulatory sequences, e.g., a 5′ untranslated region (UTR), e.g., a 5′UTR from a STAT3 gene or from another gene, a 3′ UTR, e.g., a 3′UTR from a STAT3 gene or from another gene; a polyadenylation site; an insulator sequence. In another preferred embodiment, the level of STAT3 protein is increased by increasing the level of expression of an endogenous STAT3 gene, e.g., by increasing transcription of the STAT3 gene or increasing STAT3 mRNA stability. In a preferred embodiment, transcription of the STAT3 gene is increased by: altering the regulatory sequence of the endogenous STAT3 gene, e.g., by the addition of a positive regulatory element (such as an enhancer or a DNA-binding site for a transcriptional activator); the deletion of a negative regulatory element (such as a DNA-binding site for a transcriptional repressor) and/or replacement of the endogenous regulatory sequence, or elements therein, with that of another gene, thereby allowing the coding region of the STAT3 gene to be transcribed more efficiently.

In another preferred embodiment, the agent increases POMC and/or αMSH levels and/or activity. The agent can be, e.g.: a POMC polypeptide or a functional fragment or analog thereof; an αMSH peptide or functional fragment or analog thereof; a peptide or protein that increases POMC activity, e.g., a peptide or protein that increases formation of αMSH from POMC; a small molecule that increases expression of POMC, e.g., by binding to the promoter region of the POMC gene; an antibody, e.g., an antibody that binds to and stabilizes or assists the binding of POMC or αMSH to a POMC or αMSH binding partner (e.g., an MC-3 or MC-4 polypeptide); or a nucleotide sequence encoding a POMC polypeptide or functional fragment or analog thereof. The nucleotide sequence can be a genomic sequence or a cDNA sequence. The nucleotide sequence can include: a POMC coding region; a promoter sequence, e.g., a promoter sequence from a POMC gene or from another gene; an enhancer sequence; untranslated regulatory sequences, e.g., a 5′ untranslated region (UTR), e.g., a 5′UTR from a POMC gene or from another gene, a 3′ UTR, e.g., a 3′UTR from a POMC gene or from another gene; a polyadenylation site; an insulator sequence. In another preferred embodiment, the level of POMC protein is increased by increasing the level of expression of an endogenous POMC gene, e.g., by increasing transcription of the POMC gene or increasing POMC mRNA stability. In a preferred embodiment, transcription of the POMC gene is increased by: altering the regulatory sequence of the endogenous POMC gene, e.g., by the addition of a positive regulatory element (such as an enhancer or a DNA-binding site for a transcriptional activator); the deletion of a negative regulatory element (such as a DNA-binding site for a transcriptional repressor) and/or replacement of the endogenous regulatory sequence, or elements therein, with that of another gene, thereby allowing the coding region of the POMC gene to be transcribed more efficiently.

In another preferred embodiment, the agent increases MC-3 and/or MC-4 levels or activity. The agent can be, e.g.: a MC-3 and/or MC-4 polypeptide or a functional fragment or analog thereof; a peptide or protein that increases MC-3 or MC-4 activity, e.g., a peptide or protein that binds and activates MC-3 and/or MC-4; a small molecule that increases expression of MC-3 and/or MC-4, e.g., by binding to the promoter region of the MC-3 and/or MC-4 gene; an antibody, e.g., an antibody that binds to and stabilizes or assists the binding of MC-3 and/or MC-4 to a MC-3 and/or MC-4 binding partner, e.g., αMSH; or a nucleotide sequence encoding a MC-3 and/or MC-4 polypeptide or functional fragment or analog thereof. The nucleotide sequence can be a genomic sequence or a cDNA sequence. The nucleotide sequence can include: a MC-3 and/or MC-4 coding region; a promoter sequence, e.g., a promoter sequence from a MC-3 or MC-4 gene or from another gene; an enhancer sequence; untranslated regulatory sequences, e.g., a 5′ untranslated region (UTR), e.g., a 5′UTR from a MC-3 and/or MC-4 gene or from another gene, a 3′ UTR, e.g., a 3′UTR from a MC-3 and/or MC-4 gene or from another gene; a polyadenylation site; an insulator sequence. In another preferred embodiment, the level of MC-3 and/or MC-4 protein is increased by increasing the level of expression of an endogenous MC-3 and/or MC-4 gene, e.g., by increasing transcription of the MC-3 and/or MC-4 gene or increasing MC-3 and/or MC-4 mRNA stability. In a preferred embodiment, transcription of the MC-3 and/or MC-4 gene is increased by: altering the regulatory sequence of the endogenous MC-3 and/or MC-4 gene, e.g., by the addition of a positive regulatory element (such as an enhancer or a DNA-binding site for a transcriptional activator); the deletion of a negative regulatory element (such as a DNA-binding site for a transcriptional repressor) and/or replacement of the endogenous regulatory sequence, or elements therein, with that of another gene, thereby allowing the coding region of the MC-3 and/or MC-4 gene to be transcribed more efficiently.

In a preferred embodiment, a pharmaceutical composition including one or more of the agents described herein is administered in a pharmaceutically effective dose.

In a preferred embodiment, the administration of an agent which promotes LRb-STAT3 signaling can be initiated: when the subject begins to show signs of unwanted eating behavior or gain in weight, e.g., as evidenced by an increase of more than 5, 10, 20, or 30% in body weight or when the subject is 5, 10, 20, or 30% above normal body weight; when an increase in appetite is diagnosed; at the time a treatment which promotes eating, appetite, or weight gain or maintenance, is begun or begins to exert its effects; or generally, as is needed to maintain health or acceptable weight levels.

The period over which the agent is administered (or the period over which clinically effective levels are maintained in the subject) can be long term, e.g., for six months or more or a year or more, or short term, e.g., for less than a year, six months, one month, two weeks or less.

In another aspect, LRb-STAT3 signaling is decreased, to thereby increase appetite and/or body weight and/or caloric intake. In one embodiment, the subject is under weight or exhibits less than normal eating behavior, e.g., the subject suffers from an immune system disorder (e.g., AIDS, or is HIV positive), the subject suffers from anorexia nervosa, or renal disease (e.g., chronic renal disease), or the subject is, has or will be administered a treatment which results in weight loss or decreased appetite such as chemotherapy, radiation therapy or dialysis. Preferably, body weight, appetite and/or caloric intake is increased without an effect on a reproductive function, e.g., fertility or height. In a preferred embodiment, the method includes evaluating a reproductive function of the mammal, e.g., a reproductive function described herein. In a preferred embodiment, LRb-STAT3 signaling is decreased by administering an agent that decreases an LRb-STAT3 signaling activity. An agent that decreases an LRb-STAT3 signaling activity can be one or more of: an agent that inhibits LRb Tyr¹¹³⁸ (or a corresponding tyrosine, e.g., Tyr¹¹⁴¹ of human LRb) phosphorylation; an agent that inhibits STAT3 levels and/or activity (see, e.g., Turkson at al. (2000) Oncogene 19(56):6613-26; Bowman et al. (1999) Cancer Control: Journal of the Moffett Cancer Center 6:615); an agent that inhibits proopiomelanocortin (POMC) levels and/or activity; an agent that inhibits αMSH levels and/or activity.

In a preferred embodiment, the agent inhibits or decreases phosphorylation of the tyrosine located at about amino acid 1138, e.g., LRb Tyr¹¹³⁸ in mouse or the corresponding tyrosine residue in another mammal, e.g., LRb Tyr¹¹⁴¹ in human. The agent can be, e.g., a phosphatase that dephosphorylates a phosphorylated Tyr¹¹³⁸ of LRb (or corresponding tyrosine, e.g., Tyr¹¹⁴¹ of human LRb); an analog of LRb that mimics dephosphorylated LRb, e.g., an LRb analog in which Tyr¹¹³⁸ (or corresponding tyrosine, e.g., Tyr¹¹⁴¹ of human LRb) is absent and/or substituted with an amino acid residue that cannot be phosphorylated; a kinase antagonist, e.g., a Jak2 antagonist that decreases Jak2 kinase activity, and agent that binds to LRb such that phosphorylation of Tyr¹¹³⁸ (or a corresponding tyrosine, e.g., Tyr¹¹⁴¹ of human LRb) is inhibited, e.g., an antibody or peptide that binds to LRb and blocks Tyr¹¹³⁸ (or corresponding tyrosine, e.g., LRb Tyr¹¹⁴¹ of human LRb) phosphorylation of LRb, e.g., a Stat3 SH2 domain-binding phosphopeptide, e.g., PY*LKTK, can disrupt activated Stat3 (Turkson et al. (2001) J. Biol. Chem. 276:45443-45455).

In a preferred embodiment, the agent that inhibits STAT3 levels and/or activity can be one or more of: a STAT3 binding protein, e.g., a soluble STAT3 binding protein that binds and inhibits a STAT3 activity, e.g., dimerization activity, nuclear translocation activity, DNA binding activity, or transcriptional activation activity; an antibody that specifically binds to the STAT3 protein, e.g., an antibody that disrupts STAT3's ability to bind LRb, to dimerize, to translocate to the nucleus, or bind DNA, or blocks the phosphorylation of STAT3 (e.g., at Tyr 705 or Ser 727 of STAT3 or a corresponding tyrosine or serine); a mutated inactive STAT3 or fragment thereof which, e.g., binds to a STAT3 binding partner (e.g., LRb, another STAT3 molecule or DNA) but disrupts a STAT3 activity, e.g., dimerization, nuclear translocation activity or transcriptional activation activity (e.g., a dominant negative STAT3, see, e.g., Bowman, supra); a STAT3 nucleic acid molecule that can bind to a cellular STAT3 nucleic acid sequence, e.g., mRNA, and inhibit expression of the protein, e.g., an antisense molecule or STAT3 ribozyme; an agent which decreases STAT3 gene expression, e.g., a small molecule which binds the promoter of STAT3 and decreases STAT3 gene expression. In another preferred embodiment, STAT3 is inhibited by decreasing the level of expression of an endogenous STAT3 gene, e.g., by decreasing transcription of the STAT3 gene. In a preferred embodiment, transcription of the STAT3 gene can be decreased by: altering the regulatory sequences of the endogenous STAT3 gene, e.g., by the addition of a negative regulatory sequence (such as a DNA-biding site for a transcriptional repressor), or by the removal of a positive regulatory sequence (such as an enhancer or a DNA-binding site for a transcriptional activator).

In a preferred embodiment, the agent that inhibits POMC and/or αMSH levels and/or activity can be one or more of: a POMC or αMSH binding protein, e.g., a soluble POMC or αMSH binding protein that binds and inhibits a POMC or αMSH activity; an antibody that specifically binds to POMC or αMSH, e.g., an antibody that disrupts the ability of POMC to form αMSH or disrupts the ability of αMSH too function as a peptide hormone; a mutated inactive POMC or αMSH or fragment thereof which, e.g., binds to a αMSH binding partner (e.g., an MC3 or MC4 receptor) but disrupts an αMSH activity, e.g., αMSH neuronal signaling activity; a POMC nucleic acid molecule that can bind to a cellular POMC nucleic acid sequence, e.g., mRNA, and inhibit expression of the protein, e.g., an antisense molecule or POMC ribozyme; an agent which decreases POMC gene expression, e.g., a small molecule which binds the promoter of POMC and decreases POMC gene expression. In another preferred embodiment, POMC is inhibited by decreasing the level of expression of an endogenous POMC gene, e.g., by decreasing transcription of the POMC gene. In a preferred embodiment, transcription of the POMC gene can be decreased by: altering the regulatory sequences of the endogenous POMC gene, e.g., by the addition of a negative regulatory sequence (such as a DNA-biding site for a transcriptional repressor), or by the removal of a positive regulatory sequence (such as an enhancer or a DNA-binding site for a transcriptional activator).

In a preferred embodiment, a pharmaceutical composition including one or more of the agents described herein is administered in a therapeutically effective dose.

In preferred embodiments, the method further includes diagnosing the subject as being at risk for: a disorder or unwanted condition related to an eating, appetite, or weight-related disorder; less than normal eating behavior; wasting; or being underweight.

In another aspect, the invention features a method of modulating lactation in a mammal. The method includes: identifying a subject in need of having lactation modulated; and administering to the mammal an agent that modulates LRb-STAT3 signaling. Preferably, lactation is modulated without an effect on fertility or height. The mammal can be a primate, e.g., an ape or a human; a rodent, e.g., a rat, mouse, or an animal model for a leptin related disorder, e.g., a ob/ob mouse, a db/db mouse, a Zucker rat, or a nod mouse.

In one aspect, LRb-STAT3 signaling is promoted or increased, to thereby decrease lactation. In a preferred embodiment, LRb-STAT3 signaling is increased by administering an agent that promotes an LRb-STAT3 signaling activity. An agent that promotes an LRb-STAT3 signaling activity can be one or more of: an agent that increases or mimics LRb Tyr¹¹³⁸ (or a corresponding tyrosine, e.g., Tyr¹¹⁴¹ of human LRb) phosphorylation; an agent that increases a STAT3 levels and/or activity; an agent that increases proopiomelanocortin (POMC) levels and/or activity; an agent that increases αMSH levels or activity an agent that increases MC-3 and/or MC-4 levels and/or activity.

In a preferred embodiment, the agent increases or mimics the phosphorylation of the tyrosine located at about amino acid 1138 of LRb (e.g., mouse LRb Tyr¹¹³⁸ or the corresponding tyrosine residue in another mammal, e.g., human LRb Tyr¹¹⁴¹). The agent can be, e.g., a kinase that phosphorylates Tyr¹³⁸ or a corresponding tyrosine of LRb, e.g., Jak2 or a functional fragment or analog thereof; an analog of LRb that mimics phosphorylated LRb, e.g., an LRb analog in which Tyr¹¹³⁸ or a corresponding tyrosine, e.g., Tyr¹¹⁴¹ of human LRb, is substituted with Asp or Glu; a kinase agonist, e.g., a Jak2 agonist that increases Jak2 kinase activity.

In a preferred embodiment, the agent increases STAT3 activity. The agent can be, e.g., a STAT3 polypeptide or a functional fragment or analog thereof, preferably a transcriptionally active STAT3 polypeptide or a functional fragment or analog thereof; a peptide or protein agonist of STAT3 that increases the activity, e.g., the transcriptional activity, of STAT3 (e.g., by increasing phosphorylation of STAT3, e.g., at Tyr705 or Ser727, by promoting or stabilizing dimerization of STAT3, by increasing nuclear translocation of STAT3, by promoting or stabilizing STAT3 binding to DNA); a small molecule that increases expression of STAT3, e.g., by binding to the promoter region of the STAT3 gene; an antibody, e.g., an antibody that binds to and stabilizes or assists the binding of STAT3 to a STAT3 binding partner (e.g., LRb, another STAT3 molecule, or DNA); or a nucleotide sequence encoding a STAT3 polypeptide or functional fragment or analog thereof. The nucleotide sequence can be a genomic sequence or a cDNA sequence. The nucleotide sequence can include: a STAT3 coding region; a promoter sequence, e.g., a promoter sequence from a STAT3 gene or from another gene; an enhancer sequence; untranslated regulatory sequences, e.g., a 5′ untranslated region (UTR), e.g., a 5′UTR from a STAT3 gene or from another gene, a 3′ UTR, e.g., a 3′UTR from a STAT3 gene or from another gene; a polyadenylation site; an insulator sequence. In another preferred embodiment, the level of STAT3 protein is increased by increasing the level of expression of an endogenous STAT3 gene, e.g., by increasing transcription of the STAT3 gene or increasing STAT3 mRNA stability. In a preferred embodiment, transcription of the STAT3 gene is increased by: altering the regulatory sequence of the endogenous STAT3 gene, e.g., by the addition of a positive regulatory element (such as an enhancer or a DNA-binding site for a transcriptional activator); the deletion of a negative regulatory element (such as a DNA-binding site for a transcriptional repressor) and/or replacement of the endogenous regulatory sequence, or elements therein, with that of another gene, thereby allowing the coding region of the STAT3 gene to be transcribed more efficiently.

In another preferred embodiment, the agent increases POMC and/or αMSH levels and/or activity. The agent can be, e.g.: a POMC polypeptide or a functional fragment or analog thereof; an αMSH peptide or functional fragment or analog thereof; a peptide or protein that increases POMC activity, e.g., a peptide or protein that increases formation of αMSH from POMC; a small molecule that increases expression of POMC, e.g., by binding to the promoter region of the POMC gene; an antibody, e.g., an antibody that binds to and stabilizes or assists the binding of POMC or αMSH to a POMC or αMSH binding partner (e.g., an MC-3 and/or MC-4 polypeptide); or a nucleotide sequence encoding a POMC polypeptide or functional fragment or analog thereof. The nucleotide sequence can be a genomic sequence or a cDNA sequence. The nucleotide sequence can include: a POMC coding region; a promoter sequence, e.g., a promoter sequence from a POMC gene or from another gene; an enhancer sequence; untranslated regulatory sequences, e.g., a 5′ untranslated region (UTR), e.g., a 5′UTR from a POMC gene or from another gene, a 3′ UTR, e.g., a 3′UTR from a POMC gene or from another gene; a polyadenylation site; an insulator sequence. In another preferred embodiment, the level of POMC protein is increased by increasing the level of expression of an endogenous POMC gene, e.g., by increasing transcription of the POMC gene or increasing POMC mRNA stability. In a preferred embodiment, transcription of the POMC gene is increased by: altering the regulatory sequence of the endogenous POMC gene, e.g., by the addition of a positive regulatory element (such as an enhancer or a DNA-binding site for a transcriptional activator); the deletion of a negative regulatory element (such as a DNA-binding site for a transcriptional repressor) and/or replacement of the endogenous regulatory sequence, or elements therein, with that of another gene, thereby allowing the coding region of the POMC gene to be transcribed more efficiently.

In another aspect, LRb-STAT3 signaling is decreased, to thereby increase lactation. Preferably, lactation is increased without an effect on fertility. In a preferred embodiment, LRb-STAT3 signaling is decreased by administering an agent that decreases an LRb-STAT3 signaling activity. An agent that decreases an LRb-STAT3 signaling activity can be one or more of: an agent that inhibits LRb Tyr¹¹³⁸ (or a corresponding tyrosine, e.g., Tyr¹¹⁴¹ of human LRb) phosphorylation; an agent that inhibits STAT3 levels and/or activity; an agent that inhibits proopiomelanocortin (POMC) levels and/or activity; an agent that inhibits αMSH levels and/or activity.

In a preferred embodiment, the agent inhibits or decreases phosphorylation of the tyrosine located at about amino acid 1138, e.g., LRb Tyr¹¹³⁸ in mouse or the corresponding tyrosine residue in another mammal, e.g., LRb Tyr¹¹⁴¹ in human. The agent can be, e.g., a phosphatase that dephosphorylates a phosphorylated Tyr¹¹³⁸ (or corresponding tyrosine, e.g., Tyr₁₁₄₁ of human LRb) of LRb; an analog of LRb that mimics dephosphorylated LRb, e.g., an LRb analog in which Tyr¹¹³⁸ (or corresponding tyrosine, e.g., Tyr¹¹⁴¹ of human LRb) is absent and/or substituted with an amino acid residue that cannot be phosphorylated; a kinase antagonist, e.g., a Jak2 antagonist that decreases Jak2 kinase activity, and agent that binds to LRb such that phosphorylation of Tyr¹¹³⁸ (or a corresponding tyrosine, e.g., Tyr¹¹⁴¹ of human LRb) is inhibited, e.g., an antibody or peptide that binds to LRb and blocks Tyr¹¹³⁸, or a corresponding tyrosine, phosphorylation of LRb.

In a preferred embodiment, the agent that inhibits STAT3 levels and/or activity can be one or more of: a STAT3 binding protein, e.g., a soluble STAT3 binding protein that binds and inhibits a STAT3 activity, e.g., LRb binding activity, dimerization activity, nuclear translocation activity, DNA binding activity, or transcriptional activation activity; an antibody that specifically binds to the STAT3 protein, e.g., an antibody that disrupts STAT3's ability to bind LRb, to dimerize, to translocate to the nucleus, to bind DNA, to be phosphorylated (e.g., at Tyr705 and/or Ser727 or a corresponding tyrosine or serine of STAT3); a mutated inactive STAT3 or fragment thereof which, e.g., binds to a STAT3 binding partner (e.g., LRb, another STAT3 molecule or DNA) but disrupts a STAT3 activity, e.g., dimerization, nuclear translocation activity or transcriptional activation activity; a STAT3 nucleic acid molecule that can bind to a cellular STAT3 nucleic acid sequence, e.g., mRNA, and inhibit expression of the protein, e.g., an antisense molecule or STAT3 ribozyme; an agent which decreases STAT3 gene expression, e.g., a small molecule which binds the promoter of STAT3 and decreases STAT3 gene expression. In another preferred embodiment, STAT3 is inhibited by decreasing the level of expression of an endogenous STAT3 gene, e.g., by decreasing transcription of the STAT3 gene. In a preferred embodiment, transcription of the STAT3 gene can be decreased by: altering the regulatory sequences of the endogenous STAT3 gene, e.g., by the addition of a negative regulatory sequence (such as a DNA-biding site for a transcriptional repressor), or by the removal of a positive regulatory sequence (such as an enhancer or a DNA-binding site for a transcriptional activator).

In a preferred embodiment, the agent that inhibits POMC and/or αMSH levels and/or activity can be one or more of: a POMC or αMSH binding protein, e.g., a soluble POMC or αMSH binding protein that binds and inhibits a POMC or αMSH activity; an antibody that specifically binds to POMC or αMSH, e.g., an antibody that disrupts the ability of POMC to form αMSH or disrupts the ability of αMSH to function as a peptide hormone; a mutated inactive POMC or αMSH or fragment thereof which, e.g., binds to a αMSH binding partner (e.g., an MC3 or MC4 receptor) but disrupts an αMSH activity, e.g., αMSH neuronal signaling activity; a POMC nucleic acid molecule that can bind to a cellular POMC nucleic acid sequence, e.g., mRNA, and inhibit expression of the protein, e.g., an antisense molecule or POMC ribozyme; an agent which decreases POMC gene expression, e.g., a small molecule which binds the promoter of POMC and decreases POMC gene expression. In another preferred embodiment, POMC is inhibited by decreasing the level of expression of an endogenous POMC gene, e.g., by decreasing transcription of the POMC gene. In a preferred embodiment, transcription of the POMC gene can be decreased by: altering the regulatory sequences of the endogenous POMC gene, e.g., by the addition of a negative regulatory sequence (such as a DNA-biding site for a transcriptional repressor), or by the removal of a positive regulatory sequence (such as an enhancer or a DNA-binding site for a transcriptional activator).

In a preferred embodiment, a pharmaceutical composition including one or more of the agents described herein is administered in a therapeutically effective dose.

In another aspect, the invention features a transgenic non-human mammal, e.g., a primate, a rodent, e.g., a rat, mouse, or guinea pig, having a disruption in the LRb gene wherein the disruption causes a reduction in LRb-STAT3 signaling, but preferably not in other LRb signaling pathways (e.g., a Tyr⁹⁸⁵ mediated pathway, e.g., a pathway involving SHP-2 and/or SOCS3). The disruption in the LRb gene can be a deletion, addition, or substitution, but is preferably not a knock-out of the entire LRb gene. In one embodiment, the disruption causes the mutation, substitution, or deletion of Tyr1138 (or a corresponding tyrosine) of LRb, e.g., Tyr¹¹³⁸ is replaced with an amino acid residue other than Tyr or with a Tyr analog (e.g., a Tyr analog that is not phosphorylated). The disruption can be of a portion of the LRb gene which includes Tyr1138 (or a corresponding tyrosine), or the disruption can be of Tyr 1138 (or a corresponding tyrosine). In a preferred embodiment, Tyr985 (or a corresponding tyrosine) is unaffected.

In a preferred embodiment, the transgenic non-human mammal displays one or more of the following phenotypes: (1) it has a body weight at least 10% heavier than wild-type mammal, e.g., it is obese compared to a wild-type mammal; (2) it is hyperglycemic compared to a wild-type mammal; (3) it has elevated glucocorticoid levels compared to a wild-type mammal; (4) it has elevated leptin levels compared to a wild-type mammal; (5) it has increased whole body triglyceride levels compared to a wild-type mammal; (6) it has increased adiposity compared to a wild-type mammal; (7) it exhibits increased feeding behavior compared to a wild-type mammal; (8) it exhibits decreased energy expenditure compared to a wild-type mammal; (9) it exhibits a faster rate of weight gain compared to a wild-type mammal; (10) it is hyperinsulinemic compared to a wild-type mammal; (11) it is hyperglycemic compared to a wild-type mammal; (12) it has altered melanocortin levels compared to a wild-type mammal; (13) if a female, it has reduced ability to lactate postpartum; (14) it is fertile; (15) if a female, it undergoes estrous cycles; (16) it has a grossly normal reproductive tract; (17) it has the ability to activate ERK through LRb; (18) it has a normal length and/or height compared to a wild-type mammal.

In a preferred embodiment, the transgenic mammal is obese and fertile. In a preferred embodiment, the transgenic mammal can be used in the screening assays described herein to identify a treatment which modulates body weight, appetite and/or caloric intake, preferably without effecting fertility or height.

In a preferred embodiment, the disruption is a substitution of one or more nucleotides. In a preferred embodiment, the substitution includes a substitution of the codon encoding a tyrosine located at about amino acid 1138 of LRb, e.g., mouse LRb Tyr¹¹³⁸ or human LRb Tyr¹¹⁴¹. The substituted codon can be a codon encoding any amino acid other than Tyr, e.g., Ser, Asp or Glu, or can be a Tyr analog, e.g., a Tyr analog which is not phosphorylated.

In another preferred embodiment, the disruption is a deletion of between 3 and 300 nucleotides, more preferably between 3 and 150 nucleotides, between 3 and 90 nucleotides, between 3 and 30 nucleotides, or between 3 and 9 nucleotides. In a preferred embodiment, the deletion encompasses Tyr1138 (or a corresponding tyrosine) of LRb. Preferably, the deletion does not alter the reading frame of the LRb gene.

In a preferred embodiment, the disruption is homozygous.

In another preferred embodiment, the disruption is heterozygous.

In another aspect, the invention features a transgenic non-human mammal, e.g., a primate, a rodent, e.g., a rat, mouse, or guinea pig, having a transgene encoding human LRb, e.g., a human LRb having disruption in the LRb gene wherein the disruption causes a reduction in LRb-STAT3 signaling, but preferably not in other LRb signaling pathways (e.g., a Tyr⁹⁸⁶ mediated pathway, e.g., a pathway involving SHP-2 and/or SOCS3). The disruption in the human LRb gene can be a deletion, addition, or substitution, but is preferably not a knock-out of the entire human LRb gene. In one embodiment, the disruption causes the mutation, substitution, or deletion of Tyr1141 of LRb, e.g., Tyr¹¹⁴¹ is replaced with an amino acid residue other than Tyr or with a Tyr analog (e.g., a Tyr analog that is not phosphorylated). The disruption can be of a portion of the LRb gene which includes Tyr1141, or the disruption can be of Tyr 1141. In a preferred embodiment, Tyr986 is unaffected.

In one embodiment, the transgenic non-human mammal is capable of expressing endogenous wild-type LRb. For example, the transgenic mammal can be a mouse which is capable of expressing wild-type murine LRb. In another preferred embodiment, the transgenic non-human mammal can have a disruption in its endogenous LRb gene. For example, the transgenic mammal can have the entire endogenous LRb gene knocked out or can have a disruption in the LRb gene wherein the disruption causes a reduction in an LRb signaling pathway, e.g., in LRb-STAT3 signaling. The disruption in the endogenous LRb gene can be any disruption described herein.

In a preferred embodiment, the transgenic non-human mammal displays one or more of the following phenotypes: (1) it has a body weight at least 10% heavier than wild-type mammal, e.g., it is obese compared to a wild-type mammal; (2) it is hyperglycemic compared to a wild-type mammal; (3) it has elevated glucocorticoid levels compared to a wild-type mammal; (4) it has elevated leptin levels compared to a wild-type mammal; (5) it has increased whole body triglyceride levels compared to a wild-type mammal; (6) it has increased adiposity compared to a wild-type mammal; (7) it exhibits increased feeding behavior compared to a wild-type mammal; (8) it exhibits decreased energy expenditure compared to a wild-type mammal; (9) it exhibits a faster rate of weight gain compared to a wild-type mammal; (10) it is hyperinsulinemic compared to a wild-type mammal; (11) it is hyperglycemic compared to a wild-type mammal; (12) it has altered melanocortin levels compared to a wild-type mammal; (13) if a female, it has reduced ability to lactate post-partum; (14) it is fertile; (15) if a female, it undergoes estrous cycles; (16) it has a grossly normal reproductive tract; (17) it has the ability to activate ERK through LRb; (18) it has a normal length and/or height compared to a wild-type mammal.

In a preferred embodiment, the transgenic mammal is a knockout for its endogenous LRb gene and includes a transgene encoding a human LRb wherein the human LRb has a disruption which causes a reduction in LRb-STAT3 signaling but preferably not other LRb signaling pathways, e.g., a pathway mediated by Tyr⁹⁸⁶. In a preferred embodiment, the transgenic mammal can be used in the screening assays described herein to identify a treatment which modulates body weight, appetite and/or caloric intake, preferably without effecting fertility or height.

In a preferred embodiment, the disruption of the human Tyr is a substitution of one or more nucleotides. In a preferred embodiment, the substitution includes a substitution of the codon encoding a tyrosine located at about amino acid 1141 of LRb. The substituted codon can be a codon encoding any amino acid other than Tyr, e.g., Ser, Asp or Glu, or can be a Tyr analog, e.g., a Tyr analog which is not phosphorylated.

In another preferred embodiment, the disruption is a deletion of between 3 and 300 nucleotides, more preferably between 3 and 150 nucleotides, between 3 and 90 nucleotides, between 3 and 30 nucleotides, or between 3 and 9 nucleotides. In a preferred embodiment, the deletion encompasses Tyr1141 of LRb. Preferably, the deletion does not alter the reading frame of the LRb gene.

In a preferred embodiment, the disruption of the human Tyr1141 is homozygous.

In another preferred embodiment, the disruption of the human Tyr1141 is heterozygous.

In another aspect, the invention features a transgenic non-human mammal, e.g., a primate, a rodent, e.g., a rat, mouse, or guinea pig, having a transgene which includes a transcriptional control region from a polypeptide associated with the LRb-STAT3 pathway and a sequence encoding a protein functionally unrelated to that polypeptide, e.g., a sequence encoding a reporter molecule. In a preferred embodiment, the transcriptional control region is: a STAT3 regulatory control region; a POMC regulatory control region; a Jak3 regulatory control region, an MC-3 regulatory control region; an MC-4 regulatory control region. The regulatory control region can include a promoter or a functional fragment thereof from a polypeptide involved in the LRb-STAT3 signaling pathway. The regulatory control region can further include an enhancer sequence, an untranslated regulatory sequence, e.g., a 5′ untranslated region (UTR), from the LRb-STAT3 signaling polypeptide or from another gene.

In preferred embodiments, the transgenic mammal further includes a disruption in the gene naturally encoding the polypeptide from which the regulatory control region is derived, e.g., the mammal includes a disruption in: the STAT3 gene; the Jak3 gene; the POMC gene; the MC-3 gene; and/or the MC-4 gene. In one embodiment, the disruption is a mutation which results from, a chromosomal alteration or which results from, any of an alteration resulting from homologous recombination, site-specific recombination, nonhomologous recombination. The mutation is, or results from, any of an inversion, deletion, insertion, translocation, or reciprocal translocation or from, any of a deletion of one or more nucleotides from the gene, an addition of one or more nucleotides to the gene, a change of identity of one or more nucleotides of the gene. In one embodiment, the sequence encoding the reporter molecule can replace the sequence encoding the polypeptide of the LRb-STAT3 pathway. In another embodiment, the sequence encoding be placed within the sequence encoding the polypeptide of the LRb-STAT3 pathway such that the reporter molecule is expressed. For example, the sequence encoding the reporter molecule can be placed in front of or within (e.g., within an intron) of the sequence encoding the polypeptide of the LRb-STAT3 pathway, e.g., the sequence encoding the reporter molecule can include a stop codon at its 3′ end such that the polypeptide of the LRb-STAT3 pathway is not expressed or the sequence encoding the reporter molecule can include, e.g., an IRES sequence such that it is expressed as a fusion protein with the polypeptide of the LRb-STAT3 pathway.

In a preferred embodiment, the unrelated protein is a reporter molecule, e.g., a colored, luminescent or fluorescent molecule (e.g., green fluorescent protein (GFP) or a variant thereof or red fluorescent protein (RFP) or a variant thereof). Preferably, the reporter molecule can be detected in a live mammal, e.g., can be a fluorescent protein detectable, e.g., by a confocal microscope.

In another preferred embodiment, the transgenic mammal can further include a second transgene, e.g., a transgene which includes a transcription control region from a polypeptide associated with the LRb-ERK pathway and a sequence encoding a protein functionally unrelated to that polypeptide, e.g., a sequence encoding a reporter molecule. In a preferred embodiment, the transcriptional control region is: a ERK regulatory control region; a SHP-2 regulatory control region; a Jak3 regulatory control region, a SOCS3 regulatory control region. The regulatory control region can include a promoter or a functional fragment thereof from a polypeptide involved in the LRb-ERK signaling pathway. The regulatory control region can further include an enhancer sequence, an untranslated regulatory sequence, e.g., a 5′ untranslated region (UTR), from the LRb-ERK signaling polypeptide or from another gene. In a preferred embodiment, the reporter molecule encoded by the second transgene is a different reporter molecule than is under the control of the LRb-STAT3 transcription control region. For example, the first transgene can include a sequence encoding GFP operably linked to an LRb-STAT3 transcription control region and the second transgene can include a sequence encoding RFP operably linked to an LRb-ERK transcription control region, or visa versa.

In preferred embodiments, when the transgenic mammal includes a second transgene which includes a transcription control region from a LRb-ERK pathway and a sequence encoding a reporter molecule, the transgenic mammal can further include a disruption in the gene naturally encoding the polypeptide from which the LRb-ERK regulatory control region is derived, e.g., the mammal includes a disruption in: the ERK gene; the Jak3 gene; the SHP-2 gene; and/or the SOCS3 gene. In one embodiment, the disruption is a mutation which results from, a chromosomal alteration or which results from, any of an alteration resulting from homologous recombination, site-specific recombination, nonhomologous recombination. The mutation is, or results from, any of an inversion, deletion, insertion, translocation, or reciprocal translocation or from, any of a deletion of one or more nucleotides from the gene, an addition of one or more nucleotides to the gene, a change of identity of one or more nucleotides of the gene. In one embodiment, the sequence encoding the reporter molecule can replace the sequence encoding the polypeptide of the LRb-ERK pathway. In another embodiment, the sequence encoding be placed within the sequence encoding the polypeptide of the LRb-ERK pathway such that the reporter molecule is expressed. For example, the sequence encoding the reporter molecule can be placed in front of or within (e.g., within an intron) of the sequence encoding the polypeptide of the LRb-ERK pathway, e.g., the sequence encoding the reporter molecule can include a stop codon at its 3′ end such that the polypeptide of the LRb-ERK pathway is not expressed or the sequence encoding the reporter molecule can include, e.g., an IRES sequence such that it is expressed as a fusion protein with the polypeptide of the LRb-ERK pathway.

In a preferred embodiment, the transgenic mammal can be used in the screening assays described herein to identify a treatment which modulates body weight, appetite and/or caloric intake, preferably without effecting fertility or height, e.g., by detecting expression of the reporter molecule. In another preferred embodiment, the transgenic mammal including both the first and second transgenes can be used in the screening methods described herein to identify a treatment which modulates body weight/appetite/caloric intake, or fertility, or both. In another preferred embodiment, a cell from such a transgenic mammal can be used in the screening assays described herein.

In another aspect, the invention features a cell, e.g., a primate, a human, a rodent (e.g., a rat, mouse, or guinea pig) cell, having a disruption in the LRb gene wherein the disruption causes a reduction in LRb-STAT3 signaling within the cell but preferably not in other LRb signaling pathways (e.g., a Tyr⁹⁸⁵ mediated pathway, e.g., a pathway involving SHP-2 and/or SOCS3). The disruption in the LRb gene can be a deletion, addition, or substitution, but is preferably not a knock-out of the entire LRb gene. In one embodiment, the disruption causes the mutation, substitution, or deletion of Tyr1138 of LRb (or a corresponding tyrosine, e.g., Tyr¹¹⁴¹ of human LRb), e.g., Tyr¹¹³⁸ is replaced with an amino acid residue other than Tyr or with a Tyr analog (e.g., a Tyr analog that is not phosphorylated). The disruption can be of a portion of the LRb gene which includes Tyr1138 (or a corresponding tyrosine), or the disruption can be of Tyr 1138 (or a corresponding tyrosine). In a preferred embodiment, Tyr985 (or a corresponding tyrosine) is unaffected.

In a preferred embodiment, the cell displays one or more of the following characteristics: (1) it has elevated glucocorticoid levels compared to a wild-type cell; (2) it has elevated leptin levels compared to a wild-type cell; (3) it is hyperinsulinemic compared to a wild-type cells; (4) it is hyperglycemic compared to a wild-type cells; (4) it has altered melanocortin levels compared to a wild-type cell; (5) it has the ability to activate ERK through LRb.

In a preferred embodiment, the cell can be used in the screening assays described herein to identify a treatment which modulates body weight, appetite and/or caloric intake, preferably without effecting fertility or height.

In a preferred embodiment, the disruption is a substitution of one or more nucleotides. In a preferred embodiment, the substitution includes a substitution of the codon encoding a tyrosine located at about amino acid 1138 of LRb, e.g., mouse LRb Tyr¹¹³⁸ or human LRb Tyr¹¹⁴¹. The substituted codon can be a codon encoding any amino acid other than Tyr, e.g., Ser, Asp or Glu, or can be a Tyr analog, e.g., a Tyr analog that is not phosphorylated.

In another preferred embodiment, the disruption is a deletion of between 3 and 300 nucleotides, more preferably between 3 and 150 nucleotides, between 3 and 90 nucleotides, between 3 and 30 nucleotides, or between 3 and 9 nucleotides. In a preferred embodiment, the deletion encompasses Tyr1138 (or a corresponding tyrosine) of LRb. Preferably, the deletion does not alter the reading frame of the LRb gene.

In a preferred embodiment, the disruption is homozygous.

In another preferred embodiment, the disruption is heterozygous.

In another aspect, the invention features a method of evaluating a treatment for the ability to modulate appetite, body weight and/or caloric intake. Preferably, a treatment can be evaluated for its ability to modulate appetite, weight gain or caloric intake without modulating a reproductive function, e.g., fertility, or height. The method includes: administering a test treatment to a transgenic mammal having a disruption in the LRb gene wherein the disruption causes a reduction in LRb-STAT3 signaling (e.g., a transgenic mammal described hereinabove); and determining whether the test treatment affects appetite and/or body weight in the transgenic mammal, e.g., decreases appetite and/or weight gain.

In a preferred embodiment, determining whether the test treatment affects appetite or body weight in the transgenic mammal includes evaluating one or more of the following parameters: (1) body weight; (2) glucose levels; (3) glucocorticoid levels; (4) leptin levels; (5) whole body triglyceride levels; (6) adiposity; (7) feeding behavior; (8) energy expenditure; (9) rate of weight gain; (10) insulin levels; (11) melanocortin levels. A decrease in one or more of: body weight, glucose levels, glucocorticoid levels, leptin levels, whole body triglycerides, adiposity, feeding behavior, rate or gain, insulin levels, and mellanocortin levels, and/or an increase in energy expenditure is indicative of a compound which decreases weight and/or appetite. The method can also include evaluating one or more of: Jak2 activity; LRb tyrosine phosphorylation; the presence or absence of an interaction, e.g., binding, between LRb and STAT3; STAT3 levels, expression, or activity; POMC levels, expression, or activity; αMSH levels or activity. The method can further include evaluating one or more of: ability to lactate post-partum; fertility; presence of estrous cycles; morphology of reproductive tract; activation of ERK through LRb; animal length or height.

In a preferred embodiment, the test treatment is one or more of: an agent that increases or mimics LRb Tyr¹¹³⁸ (or a corresponding tyrosine) phosphorylation; an agent that increases STAT3 levels and/or activity; an agent that increases POMC levels or activity; an agent that increases αMSH levels or activity; an agent that increases MC-3 and/or MC-4 levels and/or activity; an agent that inhibits LRb Tyr¹¹³⁸ phosphorylation; an agent that inhibits STAT3 levels and/or activity; an agent that inhibits POMC levels or activity; an agent that inhibits αMSH levels or activity; or any agent described herein that modulates LRb-STAT3 signaling. The test agent can be, e.g., a nucleic acid (e.g., an antisense, ribozyme), a polypeptide (e.g., an antibody or antigen-binding fragment thereof), a peptide fragment, a peptidomimetic, or a small molecule (e.g., a small organic molecule with a molecular weight of less than 2000 daltons). In another preferred embodiment, the test agent is a member of a combinatorial library, e.g., a peptide or organic combinatorial library, or a natural product library. In a preferred embodiment, a plurality of test agents, e.g., library members, is tested. Preferably, the test agents of the plurality, e.g., library, share structural or functional characteristics.

In a preferred embodiment, appetite and/or body weight is decreased.

In another embodiment, appetite and/or body weight is increased

In a preferred embodiment, the transgenic mammal has a disruption, e.g., a deletion or substitution mutation, in the codon of the LRb gene encoding Tyr1138 (or a corresponding tyrosine).

In another aspect, the invention features a method of evaluating a treatment for the ability to modulate appetite, body weight and/or caloric intake. Preferably, a treatment can be evaluated for its ability to modulate appetite, weight gain or caloric intake without modulating a reproductive function, e.g., fertility, or height. The method includes: administering a test treatment to a mammal, e.g., a non-human mammal, e.g., a rodent, and determining whether the test treatment affects the LRb-STAT3 pathway, to thereby identify treatments which can modulate body weight, appetite and/or caloric intake. Preferably, the treatment affects the LRb-STAT3 pathway but does not affect the LRb-ERK pathway, thereby indicating a treatment which modulates body weight, appetite and/or caloric intake without modulating reproductive function, e.g., fertility, or height.

In a preferred embodiment, determining whether the test treatment affects body weight, appetite and/or caloric intake includes evaluating one or more of the following parameters: 1) LRb1138 (or a corresponding tyrosine) phosphorylation; 2) Jak3 expression levels and/or activity; 3) STAT3 expression levels or activity; 4) POMC expression levels or activity; 5) αMSH expression levels or activity. Preferably, the methods further includes evaluating one or more of the following parameters: 1) LRb985 (or a corresponding tyrosine) phosphorylation; 2) ERK expression levels or activity; 3) SHP-2 expression levels or activity; 4) SOCS3 expression levels or activity.

In another preferred embodiment, the method includes administering the test treatment to a transgenic non-human mammal described herein having a transgene which includes a transcriptional control region from a polypeptide associated with the LRb-STAT3 pathway and a sequence encoding a protein functionally unrelated to that polypeptide, e.g., a sequence encoding a reporter molecule. The method includes determining the level of reporter molecule expression in the presence and absence of the test compound. An increase or decrease in reporter molecule expression is indicative of a treatment which modulates body weight, appetite and/or caloric intake.

In another preferred embodiment, the transgenic mammal is a transgenic mammal described herein which further includes a second transgene, e.g., a second transgene which includes a transcriptional control region from a polypeptide associated with the LRb-ERK pathway and a sequence encoding a protein functionally unrelated to that polypeptide, e.g., a sequence encoding a reporter molecule, preferably a reporter molecule other than the reporter molecule expressed by the transcriptional control region of the LRb-STAT3 pathway polypeptide. The method can include determining the level of both reporter molecules in the absence and presence of the test treatment, to thereby determine if the treatment modulates eating behavior (body weight, appetite and/or caloric intake) or reproductive function (e.g., fertility), or both. In a preferred embodiment, this screening assay can be used to identify a treatment which modulates eating behavior without affecting reproductive function. In another preferred embodiment, this screening method can be used to identify a treatment which modulates reproductive function without affecting eating behavior. Preferably, both reporter molecules are fluorescent molecules which can be evaluated on a live mammal, e.g., using a confocal microscope. For example, one reporter molecule can be GFP or a variant thereof, and the other can be RFP or a variant thereof.

In a preferred embodiment, the test treatment is one or more of: an agent that increases or mimics LRb Tyr¹¹³⁸ (or a corresponding tyrosine) phosphorylation; an agent that increases STAT3 levels and/or activity; an agent that increases POMC levels or activity; an agent that increases αMSH levels or activity; an agent that increases MC-3 and/or MC-4 levels and/or activity; an agent that inhibits LRb Tyr¹¹³⁸ phosphorylation; an agent that inhibits STAT3 levels and/or activity; an agent that inhibits POMC levels or activity; an agent that inhibits αMSH levels or activity; any agent described herein that modulates LRb-STAT3 signaling; an agent that increases or mimics LRb Tyr⁹⁸⁵ (or a corresponding tyrosine) phosphorylation; an agent that increases ERK levels and/or activity; an agent that increases SHP-2 levels or activity; an agent that increases SOCS3 levels or activity; an agent that inhibits LRb Tyr⁹⁸⁵ phosphorylation; an agent that inhibits ERK levels and/or activity; an agent that inhibits SHP-2 levels or activity; an agent that inhibits SOCS3 levels or activity; any agent described herein that modulates LRb-ERK signaling. The test agent can be, e.g., a nucleic acid (e.g., an antisense, ribozyme), a polypeptide (e.g., an antibody or antigen-binding fragment thereof), a peptide fragment, a peptidomimetic, or a small molecule (e.g., a small organic molecule with a molecular weight of less than 2000 daltons). In another preferred embodiment, the test agent is a member of a combinatorial library, e.g., a peptide or organic combinatorial library, or a natural product library. In a preferred embodiment, a plurality of test agents, e.g., library members, is tested. Preferably, the test agents of the plurality, e.g., library, share structural or functional characteristics.

In a preferred embodiment, appetite and/or body weight is decreased.

In another embodiment, appetite and/or body weight is increased.

In another preferred embodiment, fertility and/or height is decreased In yet another preferred embodiment, fertility and/or height is increased.

In another aspect, the invention features a method of screening for agents that modulate appetite and/or body weight in a mammal. The method includes screening for agents that modulate LRb-STAT3 signaling in a cell, tissue, or subject.

In one embodiment, the method includes: providing a test cell, tissue, or subject; administering a test agent to the cell, tissue, or subject; and determining whether the test agent modulates LRb-STAT3 signaling in the cell, tissue, or subject. An agent that is found to modulate LRb-STAT3 signaling in the cell, tissue, or subject is indicative of an agent that can modulate appetite and/or body weight in a mammal.

In a preferred embodiment, the test cell, tissue, or subject is a wild-type cell, tissue or subject.

In another preferred embodiment, the cell or tissue is from a transgenic mammal described herein, or the subject is a transgenic mammal described herein.

In a preferred embodiment, the method further includes administering the test agent to an animal and determining the effect of the test agent on the animal.

In a preferred embodiment, the test agent is one or more of: a nucleic acid (e.g., an antisense, ribozyme), a polypeptide (e.g., an antibody or antigen-binding fragment thereof), a peptide fragment, a peptidomimetic, or a small molecule (e.g., a small organic molecule with a molecular weight of less than 2000 daltons). In another preferred embodiment, the test agent is a member of a combinatorial library, e.g., a peptide or organic combinatorial library, or a natural product library. In a preferred embodiment, a plurality of test agents, e.g., library members, is tested. Preferably, the test agents of the plurality, e.g., library, share structural or functional characteristics.

The effect of the test agent on LRb-STAT3 signaling in the cell, tissue or subject can be assayed by numerous methods known in the art. LRb interactions with other proteins can be assayed, e.g., by standard immunoprecipitation and protein separation techniques, e.g., using anti-LRb antibodies available commercially. STAT3 levels and/or activity can be assayed, e.g., by using an anti-STAT3 antibody, e.g., an antibody specific to activated (e.g., phosphorylated) STAT3. Such antibodies are available commercially. STAT3 dimerization or binding to LRb can be detected by standard size exclusion, size separation, or immunoprecipitation techniques. STAT3 subcellular localization can be detected, e.g., using standard immunofluorescence techniques to distinguish cytoplasmic vs. nuclear localization of STAT3. As another example, a construct comprising a nucleotide sequence encoding a STAT3 responsive regulatory element operably linked to a nucleotide sequence encoding a reporter molecule can be introduced into the test cell, tissue or subject and STAT3 transcriptional activation activity can be measured using the reporter molecule as a surrogate. Tyrosine phosphorylation, e.g., Tyr1138 phosphorylation, of LRb can be assayed, e.g., using antibodies specific for phosphotyrosine.

In a preferred embodiment, a test compound found to modulate LRb-STAT3 signaling can further be evaluated in a subject, e.g., a non-human subject. The subject can be evaluated for one or more of the following parameters: (1) body weight; (2) glucose levels; (3) glucocorticoid levels; (4) leptin levels; (5) whole body triglyceride levels; (6) adiposity; (7) feeding behavior; (8) energy expenditure; (9) rate of weight gain; (10) insulin levels; (11) melanocortin levels. The subject can also be evaluated for one or more of: ability to lactate post-partum; fertility; presence of estrous cycles; morphology of reproductive tract; activation of ERK through LRb; length or height.

In another aspect, the invention features a method of determining if a subject, e.g., a human, is at risk for obesity. The method includes: evaluating a LRb-STAT3 activity, e.g., a LRb-STAT3 activity described herein, in the subject, e.g., in a cell or tissue of the subject, and comparing the LRb-STAT3 activity in the cell or tissue of the subject to a control, e.g., a cell or tissue from a non-obese subject. A lower LRb-STAT3 activity in the subject compared to a control indicates that the subject is at risk for obesity.

In another aspect, invention features a method of determining if a subject, e.g., a human, is at risk for obesity. The method includes determining the presence or absence of a genetic lesion in the LRb gene, wherein the genetic lesion disrupts the tyrosine located at about amino acid 1138 of LRb (e.g., LRb tyr¹¹³⁸ in mouse or LRb tyr¹¹⁴¹ in human) in a biological sample. Such a genetic lesion is indicative of risk for obesity in the subject. The subject is preferably a mammal, e.g., a human.

In a preferred embodiment, the method includes detecting, in a tissue of the subject, the presence or absence of any of: a deletion of one or more nucleotides from the region of the LRb gene encoding a tyrosine located at about amino acid 1138 of LRb; an insertion of one or more nucleotides into the gene, which insertion disrupts the region of the LRb gene encoding a tyrosine located at about amino acid 1138 of LRb; a point mutation, e.g., a substitution of one or more nucleotides of the gene, wherein the substitution disrupts the region of the LRb gene encoding a tyrosine located at about amino acid 1138 of LRb; a gross chromosomal rearrangement of the gene, e.g., a translocation, inversion, duplication or deletion, wherein the gross chromosomal rearrangement disrupts the region of the LRb gene encoding a tyrosine located at about amino acid 1138 of LRb.

For example, detecting the mutation can include: (i) providing a probe/primer, e.g., a labeled probe/primer, which includes a region of nucleotide sequence which hybridizes to a sense or antisense sequence from the LRb gene region that encodes a tyrosine located at about amino acid 1138 of LRb; (ii) exposing the probe/primer to nucleic acid of the tissue; and detecting, by hybridization, e.g., in situ hybridization, of the probe/primer to the nucleic acid, the presence or absence of the genetic lesion.

Methods of the invention can be used prenatally or to determine if a subject's offspring will be at risk for a disorder.

In a preferred embodiment, the method includes determining the structure of a LRb gene, an abnormal structure of the LRb region encoding a tyrosine located at about amino acid 1138 of LRb being indicative of risk for obesity.

In a preferred embodiment, the method includes contacting a sample from the subject with an antibody to the LRb protein or a nucleic acid, which hybridizes specifically with a portion of the gene that encodes a tyrosine located at about amino acid 1138 of LRb.

In yet another aspect, the present invention features a method of modulating fertility and/or height in a mammal. The method includes modulating LRb-STAT3 signaling to thereby modulate fertility and/or height. Preferably, fertility and/or height are modulated without an effect on body weight or appetite. In one embodiment, the method includes: optionally identifying a mammal in need having its fertility and/or height modulated; and administering to the mammal an agent that modulates leptin receptor long form (LRb)-ERK signaling. The mammal can be a primate, e.g., an ape or a human; a rodent, e.g., a rat, mouse, or an animal model for a fertility disorder or height related condition.

In one aspect, the agent promotes or increases LRb-ERK signaling, to thereby increase fertility and/or height. Preferably, fertility and/or height are increased without an effect on body weight. In a preferred embodiment, LRb-ERK signaling is increased by administering an agent that promotes an LRb-ERK signaling activity. The agent that promotes an LRb-ERK signaling activity can be one or more of: an agent that increases or mimics LRb Tyr985 phosphorylation; an agent that increases ERK levels and/or activity; an agent that increases SOCS 3 levels or activity.

In a preferred embodiment, the agent increases or mimics the phosphorylation of the tyrosine located at about amino acid 985 of LRb (e.g., mouse LRb Tyr985 or the corresponding tyrosine residue in another mammal, e.g., human LRb Tyr986). The agent can be, e.g., a kinase that phosphorylates Tyr985 or a corresponding tyrosine of LRb, e.g., Jak2 or a functional fragment or analog thereof; an analog of LRb that mimics phosphorylated LRb, e.g., an LRb analog in which Tyr985 or a corresponding tyrosine is substituted with Asp or Glu; a kinase agonist, e.g., a Jak2 agonist that increases Jak2 kinase activity.

In a preferred embodiment, the agent increases SHP-2 activity. The agent can be, e.g., an SHP-2 polypeptide or a functional fragment or analog thereof, preferably a transcriptionally active SHP-2 polypeptide or a functional fragment or analog thereof; a peptide or protein agonist of SHP-2 that increases the activity of SHP-2, e.g., by increasing phosphorylation of SHP-2, and/or by promoting binding of LRb and SHP-2); a small molecule that increases expression of SHP-2, e.g., by binding to the promoter region of the SHP-2 gene; an antibody, e.g., an antibody that binds to and stabilizes or assists the binding of SHP-2 to a SHP-2 binding partner (e.g., LRb); or a nucleotide sequence encoding a SHP-2 polypeptide or functional fragment or analog thereof. The nucleotide sequence can be a genomic sequence or a cDNA sequence. The nucleotide sequence can include: a SHP-2 coding region; a promoter sequence, e.g., a promoter sequence from a SHP-2 gene or from another gene; an enhancer sequence; untranslated regulatory sequences, e.g., a 5′ untranslated region (UTR), e.g., a 5′UTR from a SHP-2 gene or from another gene, a 3′ UTR, e.g., a 3′UTR from a SHP-2 gene or from another gene; a polyadenylation site; an insulator sequence. In another preferred embodiment, the level of SHP-2 protein is increased by increasing the level of expression of an endogenous SHP-2 gene, e.g., by increasing transcription of the SHP-2 gene or increasing SHP-2 mRNA stability. In a preferred embodiment, transcription of the SHP-2 gene is increased by: altering the regulatory sequence of the endogenous SHP-2 gene, e.g., by the addition of a positive regulatory element (such as an enhancer or a DNA-binding site for a transcriptional activator); the deletion of a negative regulatory element (such as a DNA-binding site for a transcriptional repressor) and/or replacement of the endogenous regulatory sequence, or elements therein, with that of another gene, thereby allowing the coding region of the SHP-2 gene to be transcribed more efficiently.

In another preferred embodiment, the agent increases ERK levels and/or activity. The agent can be, e.g.: an ERK polypeptide or a functional fragment or analog thereof, a peptide or protein that increases ERK activity; a small molecule that increases expression of ERK, e.g., by binding to the promoter region of the ERK gene; an antibody; or a nucleotide sequence encoding an ERK polypeptide or functional fragment or analog thereof. The nucleotide sequence can be a genomic sequence or a cDNA sequence. The nucleotide sequence can include: an ERK coding region; a promoter sequence, e.g., a promoter sequence from an ERK gene or from another gene; an enhancer sequence; untranslated regulatory sequences, e.g., a 5′ untranslated region (UTR), e.g., a 5′UTR from an ERK gene or from another gene, a 3′ UTR, e.g., a 3′UTR from an ERK gene or from another gene; a polyadenylation site; an insulator sequence. In another preferred embodiment, the level of ERK protein is increased by increasing the level of expression of an endogenous ERK gene, e.g., by increasing transcription of the ERK gene or increasing ERK mRNA stability. In a preferred embodiment, transcription of the ERK gene is increased by: altering the regulatory sequence of the endogenous ERK gene, e.g., by the addition of a positive regulatory element (such as an enhancer or a DNA-binding site for a transcriptional activator); the deletion of a negative regulatory element (such as a DNA-binding site for a transcriptional repressor) and/or replacement of the endogenous regulatory sequence, or elements therein, with that of another gene, thereby allowing the coding region of the ERK gene to be transcribed more efficiently.

In another preferred embodiment, the agent increases SOCS 3 levels and/or activity. The agent can be, e.g.: a SOCS 3 polypeptide or a functional fragment or analog thereof, a peptide or protein that increases SOCS 3 activity; a small molecule that increases expression of SOCS 3, e.g., by binding to the promoter region of the SOCS 3 gene; an antibody; or a nucleotide sequence encoding a SOCS 3 polypeptide or functional fragment or analog thereof. The nucleotide sequence can be a genomic sequence or a cDNA sequence. The nucleotide sequence can include: a SOCS 3 coding region; a promoter sequence, e.g., a promoter sequence from a SOCS 3 gene or from another gene; an enhancer sequence; untranslated regulatory sequences, e.g., a 5′ untranslated region (UTR), e.g., a 5′UTR from a SOCS 3 gene or from another gene, a 3′ UTR, e.g., a 3′UTR from a SOCS 3 gene or from another gene; a polyadenylation site; an insulator sequence. In another preferred embodiment, the level of SOCS 3 protein is increased by increasing the level of expression of an endogenous SOCS 3 gene, e.g., by increasing transcription of the SOCS 3 gene or increasing SOCS 3 mRNA stability. In a preferred embodiment, transcription of the SOCS 3 gene is increased by: altering the regulatory sequence of the endogenous SOCS 3 gene, e.g., by the addition of a positive regulatory element (such as an enhancer or a DNA-binding site for a transcriptional activator); the deletion of a negative regulatory element (such as a DNA-binding site for a transcriptional repressor) and/or replacement of the endogenous regulatory sequence, or elements therein, with that of another gene, thereby allowing the coding region of the SOCS 3 gene to be transcribed more efficiently.

In another aspect, LRb-ERK signaling is decreased, to thereby decrease fertility and/or height. Preferably, fertility and/or height are decreased without an effect on body weight or appetite. In a preferred embodiment, LRb-ERK signaling is decreased by administering an agent that decreases an LRb-ERK signaling activity. The agent that decreases an LRb-ERK signaling activity can be one or more of: an agent that inhibits LRb Tyr985 (or a corresponding tyrosine) phosphorylation; an agent that inhibits SHP-2 levels and/or activity; an agent that inhibits ERK levels and/or activity; an agent that inhibits SOCS 3 levels and/or activity.

In a preferred embodiment, the agent inhibits or decreases phosphorylation of the tyrosine located at about amino acid 985, e.g., LRb Tyr985 in mouse or the corresponding tyrosine residue in another mammal, e.g., LRb Tyr986 in human. The agent can be, e.g., a phosphatase that dephosphorylates a phosphorylated Tyr985 (or corresponding tyrosine) of LRb; an analog of LRb that mimics dephosphorylated LRb, e.g., an LRb analog in which Tyr985 (or corresponding tyrosine) is absent and/or substituted with an amino acid residue that cannot be phosphorylated; a kinase antagonist, e.g., a Jak2 antagonist that decreases Jak2 kinase activity, and agent that binds to LRb such that phosphorylation of Tyr985 (or a corresponding tyrosine) is inhibited, e.g., an antibody or peptide that binds to LRb and blocks Tyr985 phosphorylation of LRb.

In a preferred embodiment, the agent that inhibits SHP-2 levels and/or activity can be one or more of: a SHP-2 binding protein, e.g., a soluble SHP-2 binding protein that binds and inhibits a SHP-2 activity, e.g., SHP-2 binding activity, SHP-2 phosphorylation; an antibody that specifically binds to the SHP-2 protein, e.g., an antibody that disrupts SHP-2's ability to bind LRb; a mutated inactive SHP-2 or fragment thereof which, e.g., binds to a SHP-2 binding partner (e.g., LRb) but disrupts a SHP-2 activity; a SHP-2 nucleic acid molecule that can bind to a cellular SHP-2 nucleic acid sequence, e.g., mRNA, and inhibit expression of the protein, e.g., an antisense molecule or SHP-2 ribozyme; an agent which decreases SHP-2 gene expression, e.g., a small molecule which binds the promoter of SHP-2 and decreases SHP-2 gene expression. In another preferred embodiment, SHP-2 is inhibited by decreasing the level of expression of an endogenous SHP-2 gene, e.g., by decreasing transcription of the SHP-2 gene. In a preferred embodiment, transcription of the SHP-2 gene can be decreased by: altering the regulatory sequences of the endogenous SHP-2 gene, e.g., by the addition of a negative regulatory sequence (such as a DNA-biding site for a transcriptional repressor), or by the removal of a positive regulatory sequence (such as an enhancer or a DNA-binding site for a transcriptional activator).

In a preferred embodiment, the agent that inhibits ERK levels and/or activity can be one or more of: an ERK binding protein, e.g., a soluble ERK binding protein that binds and inhibits an ERK activity; an antibody that specifically binds to ERK, e.g., an antibody that disrupts an ERK activity; a mutated inactive ERK or fragment thereof which, e.g., binds to an ERK binding partner but disrupts an ERK activity; an ERK nucleic acid molecule that can bind to a cellular ERK nucleic acid sequence, e.g., mRNA, and inhibit expression of the protein, e.g., an antisense molecule or ERK ribozyme; an agent which decreases ERK gene expression, e.g., a small molecule which binds the promoter of ERK and decreases ERK gene expression. In another preferred embodiment, ERK is inhibited by decreasing the level of expression of an endogenous ERK gene, e.g., by decreasing transcription of the ERK gene. In a preferred embodiment, transcription of the ERK gene can be decreased by: altering the regulatory sequences of the endogenous ERK gene, e.g., by the addition of a negative regulatory sequence (such as a DNA-biding site for a transcriptional repressor), or by the removal of a positive regulatory sequence (such as an enhancer or a DNA-binding site for a transcriptional activator).

In a preferred embodiment, the agent that inhibits SOCS3 levels and/or activity can be one or more of: a SOCS3 binding protein, e.g., a soluble SOCS3 binding protein that binds and inhibits a SOCS3 activity; an antibody that specifically binds to SOCS3, e.g., an antibody that disrupts a SOCS3 activity; a mutated inactive SOCS3 or fragment thereof which, e.g., binds to a SOCS3 binding partner but disrupts a SOCS3 activity; a SOCS3 nucleic acid molecule that can bind to a cellular SOCS3 nucleic acid sequence, e.g., mRNA, and inhibit expression of the protein, e.g., an antisense molecule or SOCS3 ribozyme; an agent which decreases SOCS3 gene expression, e.g., a small molecule which binds the promoter of SOCS3 and decreases SOCS 3 gene expression. In another preferred embodiment, SOCS3 is inhibited by decreasing the level of expression of an endogenous SOCS3 gene, e.g., by decreasing transcription of the SOCS3 gene. In a preferred embodiment, transcription of the SOCS 3 gene can be decreased by: altering the regulatory sequences of the endogenous SOCS 3 gene, e.g., by the addition of a negative regulatory sequence (such as a DNA-biding site for a transcriptional repressor), or by the removal of a positive regulatory sequence (such as an enhancer or a DNA-binding site for a transcriptional activator).

In a preferred embodiment, a pharmaceutical composition including one or more of the agents described herein is administered in a therapeutically effective dose.

In another aspect, the invention features a transgenic non-human mammal, e.g., a primate, a rodent, e.g., a rat, mouse, or guinea pig, having a disruption in the LRb gene wherein the disruption causes a reduction in LRb-ERK signaling. The disruption in the LRb gene can be a deletion, addition, or substitution, but is preferably not a knock-out of the entire LRb gene. In one embodiment, the disruption causes the mutation, substitution, or deletion of Tyr985 (or a corresponding tyrosine) of LRb. The disruption can be of a portion of the LRb gene which includes Tyr985 (or a corresponding tyrosine), or the disruption can be of Tyr 985 (or a corresponding tyrosine). In a preferred embodiment, Tyr1138 (or a corresponding tyrosine) is unaffected.

In a preferred embodiment, the transgenic non-human mammal displays one or more of the following phenotypes: (1) it is infertile; (2) it is shorter than a wild-type mammal; (3) it has decreased control of gonad function compared to a wild-type mammal; (4) it has an abnormal reproductive tract compared (e.g., it is small and/or atrophic) to a wild-type mammal; (5) in females, it has decrease or absence of estrous as compared to a wild-type mammal; (6) if female, it has a reduced number or absence of corpus lacteal as compared to a wild-type mammal; (7) it exhibits poor grooming behavior compared to a wild-type mammal; (8) if female, it has no or little evidence of ovulation; (9) it has altered NPY levels compared to a wild-type mammal; (10) it is not obese as compared to db/db animals; (11) it has normal adiposity; (12) it exhibits similar feeding behavior as compared to wild-type mammals; (13) it has the ability to activate STAT3 through LRb.

In a preferred embodiment, the transgenic mammal is not obese and is infertile.

In a preferred embodiment, the disruption is a substitution of one or more nucleotides. In a preferred embodiment, the substitution includes a substitution of the codon encoding a tyrosine located at about amino acid 985 of LRb, e.g., mouse LRb Tyr⁹⁸⁵ or human LRb Tyr⁹⁸⁶. The substituted codon can be a codon encoding any amino acid other than Tyr, e.g., Ser, Asp or Glu.

In another preferred embodiment, the disruption is a deletion of between 3 and 300 nucleotides, more preferably between 3 and 150 nucleotides, between 3 and 90 nucleotides, between 3 and 30 nucleotides, or between 3 and 9 nucleotides. In a preferred embodiment, the deletion encompasses Tyr985 (or a corresponding tyrosine) of LRb. Preferably, the deletion does not alter the reading frame of the LRb gene.

In a preferred embodiment, the disruption is homozygous.

In another preferred embodiment, the disruption is heterozygous.

In another aspect, the invention features a transgenic non-human mammal, e.g., a primate, a rodent, e.g., a rat, mouse, or guinea pig, having a transgene encoding human LRb, e.g., a human LRb having disruption in the LRb gene wherein the disruption causes a reduction in LRb-ERK signaling, but preferably not in other LRb signaling pathways (e.g., a Tyr¹¹⁴¹ mediated pathway, e.g., a pathway involving STAT3 and/or POMC). The disruption in the human LRb gene can be a deletion, addition, or substitution, but is preferably not a knock-out of the entire human LRb gene. In one embodiment, the disruption causes the mutation, substitution, or deletion of Tyr986 of LRb, e.g., Tyr⁹⁸⁶ is replaced with an amino acid residue other than Tyr or with a Tyr analog (e.g., a Tyr analog that is not phosphorylated). The disruption can be of a portion of the LRb gene which includes Tyr986, or the disruption can be of Tyr 986. In a preferred embodiment, Tyr1141 is unaffected.

In one embodiment, the transgenic non-human mammal is capable of expressing endogenous wild-type LRb. For example, the transgenic mammal can be a mouse which is capable of expressing wild-type murine LRb. In another preferred embodiment, the transgenic non-human mammal can have a disruption in its endogenous LRb gene. For example, the transgenic mammal can have the entire endogenous LRb gene knocked out or can have a disruption in the LRb gene wherein the disruption causes a reduction in an LRb signaling pathway, e.g., in LRb-ERK signaling. The disruption in the endogenous LRb gene can be any disruption described herein.

In a preferred embodiment, the transgenic non-human mammal displays one or more of the following phenotypes: (1) it is infertile; (2) it is shorter than a wild-type mammal; (3) it has decreased control of gonad function compared to a wild-type mammal; (4) it has an abnormal reproductive tract compared (e.g., it is small and/or atrophic) to a wild-type mammal; (5) in females, it has decrease or absence of estrous as compared to a wild-type mammal; (6) if female, it has a reduced number or absence of corpus lacteal as compared to a wild-type mammal; (7) it exhibits poor grooming behavior compared to a wild-type mammal; (8) if female, it has no or little evidence of ovulation; (9) it has altered NPY levels compared to a wild-type mammal; (10) it is not obese as compared to db/db animals; (11) it has normal adiposity; (12) it exhibits similar feeding behavior as compared to wild-type mammals; (13) it has the ability to activate STAT3 through LRb.

In a preferred embodiment, the transgenic mammal is a knockout for its endogenous LRb gene and includes a transgene encoding a human LRb wherein the human LRb has a disruption which causes a reduction in LRb-ERK signaling but preferably not other LRb signaling pathways, e.g., a pathway mediated by Tyr1138. In a preferred embodiment, the transgenic mammal can be used in the screening assays described herein to identify a treatment which modulates reproductive function (e.g., fertility) or height, preferably without effecting eating behavior (e.g., body weight, appetite and/or caloric intake).

In a preferred embodiment, the disruption of the human Tyr is a substitution of one or more nucleotides. In a preferred embodiment, the substitution includes a substitution of the codon encoding a tyrosine located at about amino acid 986 of LRb. The substituted codon can be a codon encoding any amino acid other than Tyr, e.g., Ser, Asp or Glu, or can be a Tyr analog, e.g., a Tyr analog which is not phosphorylated.

In another preferred embodiment, the disruption is a deletion of between 3 and 300 nucleotides, more preferably between 3 and 150 nucleotides, between 3 and 90 nucleotides, between 3 and 30 nucleotides, or between 3 and 9 nucleotides. In a preferred embodiment, the deletion encompasses Tyr986 of LRb. Preferably, the deletion does not alter the reading frame of the LRb gene.

In a preferred embodiment, the disruption of the human Tyr986 is homozygous.

In another preferred embodiment, the disruption of the human Tyr986 is heterozygous.

In another aspect, the invention features a transgenic non-human mammal, e.g., a primate, a rodent, e.g., a rat, mouse, or guinea pig, having a transgene which includes a transcriptional control region from a polypeptide associated with the LRb-ERK pathway and a sequence encoding a protein functionally unrelated to that polypeptide, e.g., a sequence encoding a reporter molecule. In a preferred embodiment, the transcriptional control region is: a ERK regulatory control region; an SHP-2 regulatory control region; a Jak3 regulatory control region, a SOCS3 regulatory control region. The regulatory control region can include a promoter or a functional fragment thereof from a polypeptide involved in the LRb-ERK signaling pathway. The regulatory control region can further include an enhancer sequence, an untranslated regulatory sequence, e.g., a 5′ untranslated region (UTR), from the LRb-ERK signaling polypeptide or from another gene.

In preferred embodiments, the transgenic mammal further includes a disruption in the gene naturally encoding the polypeptide from which the regulatory control region is derived, e.g., the mammal includes a disruption in: the ERK gene; the Jak3 gene; the SHP-2 gene; and/or the SOCS3 gene. In one embodiment, the disruption is a mutation which results from, a chromosomal alteration or which results from, any of an alteration resulting from homologous recombination, site-specific recombination, nonhomologous recombination. The mutation is, or results from, any of an inversion, deletion, insertion, translocation, or reciprocal translocation or from, any of a deletion of one or more nucleotides from the gene, an addition of one or more nucleotides to the gene, a change of identity of one or more nucleotides of the gene. In one embodiment, the sequence encoding the reporter molecule can replace the sequence encoding the polypeptide of the LRb-ERK pathway. In another embodiment, the sequence encoding be placed within the sequence encoding the polypeptide of the LRb-ERK pathway such that the reporter molecule is expressed. For example, the sequence encoding the reporter molecule can be placed in front of or within (e.g., within an intron) of the sequence encoding the polypeptide of the LRb-ERK pathway, e.g., the sequence encoding the reporter molecule can include a stop codon at its 3′ end such that the polypeptide of the LRb-ERK pathway is not expressed or the sequence encoding the reporter molecule can include, e.g., an IRES sequence such that it is expressed as a fusion protein with the polypeptide of the LRb-ERK pathway.

In a preferred embodiment, the unrelated protein is a reporter molecule, e.g., a colored, luminescent or fluorescent molecule (e.g., green fluorescent protein (GFP) or a variant thereof or red fluorescent protein (RFP) or a variant thereof). Preferably, the reporter molecule can be detected in a live mammal, e.g., can be a fluorescent protein detectable, e.g., by a confocal microscope.

In another preferred embodiment, the transgenic mammal can further include a second transgene, e.g., a transgene which includes a transcription control region from a polypeptide associated with the LRb-STAT3 pathway and a sequence encoding a protein functionally unrelated to that polypeptide, e.g., a sequence encoding a reporter molecule. In a preferred embodiment, the transcriptional control region is: a STAT3 regulatory control region; a POMC regulatory control region; a Jak3 regulatory control region; a MC-3 regulatory control region; a MC-4 regulatory control region. The regulatory control region can include a promoter or a functional fragment thereof from a polypeptide involved in the LRb-STAT3 signaling pathway. The regulatory control region can further include an enhancer sequence, an untranslated regulatory sequence, e.g., a 5′ untranslated region (UTR), from the LRb-STAT3 signaling polypeptide or from another gene. In a preferred embodiment, the reporter molecule encoded by the second transgene is a different reporter molecule than is under the control of the LRb-ERK transcription control region. For example, the first transgene can include a sequence encoding GFP operably linked to an LRb-ERK transcription control region and the second transgene can include a sequence encoding RFP operably linked to an LRb-STAT3 transcription control region, or visa versa.

In preferred embodiments, when the transgenic mammal includes a second transgene which includes a transcription control region from a LRb-STAT3 pathway and a sequence encoding a reporter molecule, the transgenic mammal can further include a disruption in the gene naturally encoding the polypeptide from which the LRb-STAT3 regulatory control region is derived, e.g., the mammal includes a disruption in: the STAT3 gene; the Jak3 gene; the POMC gene; the MC-3 gene and/or the MC-4 gene. In one embodiment, the disruption is a mutation which results from, a chromosomal alteration or which results from, any of an alteration resulting from homologous recombination, site-specific recombination, nonhomologous recombination. The mutation is, or results from, any of an inversion, deletion, insertion, translocation, or reciprocal translocation or from, any of a deletion of one or more nucleotides from the gene, an addition of one or more nucleotides to the gene, a change of identity of one or more nucleotides of the gene. In one embodiment, the sequence encoding the reporter molecule can replace the sequence encoding the polypeptide of the LRb-STAT3 pathway. In another embodiment, the sequence encoding be placed within the sequence encoding the polypeptide of the LRb-STAT3 pathway such that the reporter molecule is expressed. For example, the sequence encoding the reporter molecule can be placed in front of or within (e.g., within an intron) of the sequence encoding the polypeptide of the LRb-STAT3 pathway, e.g., the sequence encoding the reporter molecule can include a stop codon at its 3′ end such that the polypeptide of the LRb-STAT3 pathway is not expressed or the sequence encoding the reporter molecule can include, e.g., an IRES sequence such that it is expressed as a fusion protein with the polypeptide of the LRb-STAT3 pathway.

In a preferred embodiment, the transgenic mammal can be used in the screening assays described herein to identify a treatment which reproductive function (e.g., fertility) and/or height, preferably without effecting eating behavior (e.g., body weight, appetite and/or caloric intake), e.g., by detecting expression of the reporter molecule. In another preferred embodiment, the transgenic mammal including both the first and second transgenes can be used in the screening methods described herein to identify a treatment which modulates reproductive function, eating behavior, or both. In another preferred embodiment, a cell from such a mammal can be used in screening assays described herein.

In another aspect, the invention features a cell, e.g., a primate, a human, a rodent (e.g., a rat, mouse, or guinea pig) cell, having a disruption in the LRb gene wherein the disruption causes a reduction in LRb-ERK signaling within the cell but preferably not in other LRb signaling pathways (e.g., a Tyr¹¹³⁸ mediated pathway, e.g., a pathway involving STAT3 and/or POMC). The disruption in the LRb gene can be a deletion, addition, or substitution, but is preferably not a knock-out of the entire LRb gene. In one embodiment, the disruption causes the mutation, substitution, or deletion of Tyr985 of LRb (or a corresponding tyrosine, e.g., Tyr⁹⁸⁶ of human LRb), e.g., Tyr⁹⁸⁵ is replaced with an amino acid residue other than Tyr or with a Tyr analog (e.g., a Tyr analog that is not phosphorylated). The disruption can be of a portion of the LRb gene which includes Tyr985 (or a corresponding tyrosine), or the disruption can be of Tyr 985 (or a corresponding tyrosine). In a preferred embodiment, Tyr1138 (or a corresponding tyrosine) is unaffected.

In a preferred embodiment, the cell displays one or more of the following characteristics: (1) it has altered hormone levels, e.g., hormones involved in gonad function, e.g., ovulation; (2) it has altered NPY levels compared to a wild-type mammal; (3) it has the ability to activate STAT3 through LRb.

In a preferred embodiment, the cell can be used in the screening assays described herein to identify a treatment which modulates reproductive function (e.g., fertility), preferably without effecting eating behavior (e.g., body weight, appetite and/or caloric intake).

In a preferred embodiment, the disruption is a substitution of one or more nucleotides. In a preferred embodiment, the substitution includes a substitution of the codon encoding a tyrosine located at about amino acid 985 of LRb, e.g., mouse LRb Tyr⁹⁸⁵ or human LRb Tyr⁹⁸⁶. The substituted codon can be a codon encoding any amino acid other than Tyr, e.g., Ser, Asp or Glu, or can be a Tyr analog, e.g., a Tyr analog that is not phosphorylated.

In another preferred embodiment, the disruption is a deletion of between 3 and 300 nucleotides, more preferably between 3 and 150 nucleotides, between 3 and 90 nucleotides, between 3 and 30 nucleotides, or between 3 and 9 nucleotides. In a preferred embodiment, the deletion encompasses Tyr985 (or a corresponding tyrosine) of LRb. Preferably, the deletion does not alter the reading frame of the LRb gene.

In a preferred embodiment, the disruption is homozygous.

In another preferred embodiment, the disruption is heterozygous.

In yet another aspect, the invention features a method of evaluating a treatment for the ability to modulate fertility and/or height. Preferably, a treatment can be evaluated for its ability to modulate a reproductive function, e.g., infertility, and/or height without modulating appetite and/or weight gain. The method includes: administering a test treatment to a transgenic mammal having a disruption in the LRb gene wherein the disruption causes a reduction in LRb-ERK signaling (e.g., a transgenic mammal described hereinabove); and determining whether the test treatment affects fertility and/or height in the transgenic mammal, e.g., increased fertility and/or height.

In a preferred embodiment, determining whether the test treatment affects fertility in the transgenic mammal includes evaluating one or more of the following parameters: (1) ovulation; (2) number of corpus lacteal; (3) gross examination of the reproductive tract; (4) NPY levels. An increase in one or more of these parameters is indicative of a compound which increases fertility. The method can also include evaluating one or more of: Jak2 activity; LRb tyrosine phosphorylation; the presence or absence of an interaction, e.g., binding, between LRb and SHP-2; ERK levels, expression, or activity; SOCS3 levels, expression, or activity. The method can further include evaluating one or more of: eating behavior; activation of STAT3 through LRb; animal weight and/or adiposity.

In a preferred embodiment, the test treatment is one or more of: an agent that increases or mimics LRb Tyr⁹⁸⁵ (or a corresponding tyrosine) phosphorylation; an agent that increases SHP-2 levels and/or activity; an agent that increases ERK levels or activity; an agent that increases SOCS3 levels or activity; an agent that inhibits LRb Tyr⁹⁸⁵ phosphorylation; an agent that inhibits SHP-2 levels and/or activity; an agent that inhibits ERK levels or activity; an agent that inhibits SOCS3 levels or activity; or any agent described herein that modulates LRb-ERK signaling. The test agent can be, e.g., a nucleic acid (e.g., an antisense, ribozyme), a polypeptide (e.g., an antibody or antigen-binding fragment thereof), a peptide fragment, a peptidomimetic, or a small molecule (e.g., a small organic molecule with a molecular weight of less than 2000 daltons). In another preferred embodiment, the test agent is a member of a combinatorial library, e.g., a peptide or organic combinatorial library, or a natural product library. In a preferred embodiment, a plurality of test agents, e.g., library members, is tested. Preferably, the test agents of the plurality, e.g., library, share structural or functional characteristics.

In a preferred embodiment, fertility and/or height are increased.

In another embodiment, fertility and/or height is decreased.

In a preferred embodiment, the transgenic mammal has a disruption, e.g., a deletion or substitution mutation, in the codon of the LRb gene encoding Tyr985 (or a corresponding tyrosine).

In another aspect, the invention features a method of evaluating a treatment for the ability to modulate reproductive function and/or height. Preferably, a treatment can be evaluated for its ability to modulate reproductive function and/or height without modulating an eating behavior (e.g., body weight, appetite, and/or caloric intake). The method includes: administering a test treatment to a mammal, e.g., a non-human mammal, e.g., a rodent, and determining whether the test treatment affects the LRb-ERK pathway, to thereby identify treatments which can modulate reproductive function and/or height. Preferably, the treatment affects the LRb-ERK pathway but does not affect the LRb-STAT3 pathway, thereby indicating a treatment which modulates reproductive function and/or height without modulating eating behavior, e.g., body weight, appetite and/or caloric intake.

In a preferred embodiment, determining whether the test treatment affects reproductive function and/or weight includes evaluating one or more of the following parameters: 1) LRb985 (or a corresponding tyrosine) phosphorylation; 2) Jak3 expression levels and/or activity; 3) ERK expression levels or activity; 4) SHP-2 expression levels or activity; 5) SOCS3 expression levels or activity. Preferably, the methods further includes evaluating one or more of the following parameters: 1) LRb1138 (or a corresponding tyrosine) phosphorylation; 2) STAT3 expression levels or activity; 3) POMC expression levels or activity; 4) αMSH expression levels or activity.

In another preferred embodiment, the method includes administering the test treatment to a transgenic non-human mammal described herein having a transgene which includes a transcriptional control region from a polypeptide associated with the LRb-ERK pathway and a sequence encoding a protein functionally unrelated to that polypeptide, e.g., a sequence encoding a reporter molecule. The method includes determining the level of reporter molecule expression in the presence and absence of the test compound. An increase or decrease in reporter molecule expression is indicative of a treatment which modulates reproductive function and/or height.

In another preferred embodiment, the transgenic mammal is a transgenic mammal described herein which further includes a second transgene, e.g., a second transgene which includes a transcriptional control region from a polypeptide associated with the LRb-STAT3 pathway and a sequence encoding a protein functionally unrelated to that polypeptide, e.g., a sequence encoding a reporter molecule, preferably a reporter molecule other than the reporter molecule expressed by the transcriptional control region of the LRb-ERK pathway polypeptide. The method can include determining the level of both reporter molecules in the absence and presence of the test treatment, to thereby determine if the treatment modulates reproductive function (e.g., fertility), or eating behavior (body weight, appetite and/or caloric intake), or both. In a preferred embodiment, this screening assay can be used to identify a treatment which modulates reproductive function without affecting eating behavior. In another preferred embodiment, this screening method can be used to identify a treatment which modulates eating behavior without reproductive function. Preferably, both reporter molecules are fluorescent molecules which can be evaluated on a live mammal, e.g., using a confocal microscope. For example, one reporter molecule can be GFP or a variant thereof, and the other can be RFP or a variant thereof.

In a preferred embodiment, the test treatment is one or more of: an agent that increases or mimics LRb Tyr⁹⁸⁵ (or a corresponding tyrosine) phosphorylation; an agent that increases ERK levels and/or activity; an agent that increases SHP-2 levels or activity; an agent that increases SOCS3 levels or activity; an agent that inhibits LRb Tyr⁹⁸⁵ phosphorylation; an agent that inhibits ERK levels and/or activity; an agent that inhibits SHP-2 levels or activity; an agent that inhibits SOCS3 levels or activity; any agent described herein that modulates LRb-ERK signaling; an agent that increases or mimics LRb Tyr¹¹³⁸ (or a corresponding tyrosine) phosphorylation; an agent that increases STAT3 levels and/or activity; an agent that increases POMC levels or activity; an agent that increases αMSH levels or activity; an agent that increases MC-3 and/or MC-4 levels and/or activity; an agent that inhibits LRb Tyr¹¹³⁸ phosphorylation; an agent that inhibits STAT3 levels and/or activity; an agent that inhibits POMC levels or activity; an agent that inhibits αMSH levels or activity; any agent described herein that modulates LRb-STAT3 signaling. The test agent can be, e.g., a nucleic acid (e.g., an antisense, ribozyme), a polypeptide (e.g., an antibody or antigen-binding fragment thereof), a peptide fragment, a peptidomimetic, or a small molecule (e.g., a small organic molecule with a molecular weight of less than 2000 daltons). In another preferred embodiment, the test agent is a member of a combinatorial library, e.g., a peptide or organic combinatorial library, or a natural product library. In a preferred embodiment, a plurality of test agents, e.g., library members, is tested. Preferably, the test agents of the plurality, e.g., library, share structural or functional characteristics.

In preferred embodiment, fertility and/or height is decreased

In another preferred embodiment, fertility and/or height is increased.

In a preferred embodiment, appetite and/or body weight is decreased.

In yet another embodiment, appetite and/or body weight is increased.

In another aspect, the invention features a method of screening for agents that modulate fertility and/or body height in a mammal. The method includes screening for agents that modulate LRb-ERK signaling in a cell, tissue, or subject.

In one embodiment, the method includes: providing a test cell, tissue, or subject; administering a test agent to the cell, tissue, or subject; and determining whether the test agent modulates LRb-ERK signaling in the cell, tissue, or subject. An agent that is found to modulate LRb-ERK signaling in the cell, tissue, or subject is indicative of an agent that can modulate fertility and/or body height in a mammal.

In a preferred embodiment, the test cell, tissue, or subject is a wild-type cell, tissue or subject.

In another preferred embodiment, the cell or tissue is from a transgenic mammal described herein, or the subject is a transgenic mammal described herein.

In a preferred embodiment, the method further includes administering the test agent to an animal and determining the effect of the test agent on the animal.

In a preferred embodiment, the test agent is one or more of: a nucleic acid (e.g., an antisense, ribozyme), a polypeptide (e.g., an antibody or antigen-binding fragment thereof), a peptide fragment, a peptidomimetic, or a small molecule (e.g., a small organic molecule with a molecular weight of less than 2000 daltons). In another preferred embodiment, the test agent is a member of a combinatorial library, e.g., a peptide or organic combinatorial library, or a natural product library. In a preferred embodiment, a plurality of test agents, e.g., library members, is tested. Preferably, the test agents of the plurality, e.g., library, share structural or functional characteristics.

The effect of the test agent on LRb-ERK signaling in the cell, tissue or subject can be assayed by numerous methods known in the art. LRb interactions with other proteins can be assayed, e.g., by standard immunoprecipitation and protein separation techniques, e.g., using anti-LRb antibodies available commercially. SHP-2 levels and/or activity can be assayed, e.g., by using an anti-SHP-2 antibody, e.g., an antibody specific to activated (e.g., phosphorylated) SHP-2. SHP-2 binding to LRb can be detected by standard size exclusion, size separation, or immunoprecipitation techniques. As another example, a construct comprising a nucleotide sequence encoding a SHP-2 responsive regulatory element operably linked to a nucleotide sequence encoding a reporter molecule can be introduced into the test cell, tissue or subject and SHP-2 transcriptional activation activity can be measured using the reporter molecule as a surrogate. Similar methods can be used to evaluate ERK and/or SOCS expression and/or activity. Tyrosine phosphorylation, e.g., Tyr985 phosphorylation, of LRb can be assayed, e.g., using antibodies specific for phosphotyrosine.

In a preferred embodiment, a test compound found to modulate LRb-ERK signaling can further be evaluated in a subject, e.g., a non-human subject. The subject can be evaluated for one or more of the following parameters: (1) ovulation; (2) number of corpus lacteal; (3) gross examination of the reproductive tract; (4) NPY levels. The method can further include evaluating one or more of: eating behavior; activation of STAT3 through LRb; animal weight and/or adiposity.

In another aspect, the invention features a method of determining if a subject, e.g., a human, is at risk for infertility. The method includes: evaluating a LRb-ERK activity, e.g., a LRb-ERK activity described herein, in the subject, e.g., in a cell or tissue of the subject, and comparing the LRb-ERK activity in the cell or tissue of the subject to a control, e.g., a cell or tissue from a fertile subject. A lower LRb-ERK activity in the subject compared to a control indicates that the subject is at risk for infertility.

In another aspect, invention features a method of determining if a subject, e.g., a human, is at risk for infertility. The method includes determining the presence or absence of a genetic lesion in the LRb gene, wherein the genetic lesion disrupts the tyrosine located at about amino acid 985 of LRb (e.g., LRb tyr⁹⁸⁵ in mouse or LRb tyr⁹⁸⁶ in human) in a biological sample. Such a genetic lesion is indicative of risk for infertility in the subject. The subject is preferably a mammal, e.g., a human.

In a preferred embodiment, the method includes detecting, in a tissue of the subject, the presence or absence of any of: a deletion of one or more nucleotides from the region of the LRb gene encoding a tyrosine located at about amino acid 985 of LRb; an insertion of one or more nucleotides into the gene, which insertion disrupts the region of the LRb gene encoding a tyrosine located at about amino acid 985 of LRb; a point mutation, e.g., a substitution of one or more nucleotides of the gene, wherein the substitution disrupts the region of the LRb gene encoding a tyrosine located at about amino acid 985 of LRb; a gross chromosomal rearrangement of the gene, e.g., a translocation, inversion, duplication or deletion, wherein the gross chromosomal rearrangement disrupts the region of the LRb gene encoding a tyrosine located at about amino acid 985 of LRb.

For example, detecting the mutation can include: (i) providing a probe/primer, e.g., a labeled probe/primer, which includes a region of nucleotide sequence which hybridizes to a sense or antisense sequence from the LRb gene region that encodes a tyrosine located at about amino acid 985 of LRb; (ii) exposing the probe/primer to nucleic acid of the tissue; and detecting, by hybridization, e.g., in situ hybridization, of the probe/primer to the nucleic acid, the presence or absence of the genetic lesion.

Methods of the invention can be used prenatally or to determine if a subject's offspring will be at risk for a disorder.

In a preferred embodiment, the method includes determining the structure of a LRb gene, an abnormal structure of the LRb region encoding a tyrosine located at about amino acid 985 of LRb being indicative of risk for infertility.

In a preferred embodiment, the method includes contacting a sample from the subject with an antibody to the LRb protein or a nucleic acid, which hybridizes specifically with a portion of the gene that encodes a tyrosine located at about amino acid 985 of LRb.

In another aspect, the invention features an LRb amino acid sequence wherein at least one amino acid has been altered such that the LRb polypeptide has an affect on eating behavior, e.g., body weight, appetite and/or caloric intake but not on reproductive function, e.g., fertility. In one embodiment, the amino acid sequence is a murine LRb amino acid sequence, e.g., the amino acid sequence of SEQ ID NO:2, wherein the amino acid residue at position 1138 has been altered, but the amino acid position 985 has not been altered. In another embodiment, the amino acid sequence is a human LRb amino acid sequence, e.g., the amino acid sequence of SEQ ID NO:4, wherein the amino acid residue at position 1141 has been altered, but the amino acid position 986 has not been altered. The alteration in the LRb amino acid sequence can be a deletion, addition, or substitution which encompasses amino acid residue 1138 of murine LRb or amino acid residue 1141 of human LRb, or a corresponding tyrosine residue in LRb from other species. In one embodiment, the alteration of the LRb amino acid sequence can be a substitution of one or more amino acids (e.g., at least one but not more than 20, 15, 10, 5 amino acids). In a preferred embodiment, the substitution includes a substitution of the amino acid residue at position 1138 of SEQ ID NO:2 or the amino acid residue at position 1141 of SEQ ID NO:4, or a corresponding tyrosine in other species. The substituted amino acid at position 1138 of SEQ ID NO:2 or position 1141 of SEQ ID NO:4, or a corresponding tyrosine in other species can be any amino acid other than Tyr, e.g., Ser, Asp or Glu, or can be a Tyr analog, e.g., a Tyr analog which is not phosphorylated or a Tyr analog which remains phosphorylated. Examples of Tyr analogs include phosphotyrosine and analogs of phosphotyrosine having a hydrolysis resistant phosphorous moiety, e.g. phosphonomethylphenylalanine (Pmp), or a hydrolysis resistant phosphorous moiety which is more electronegative than the phosphate group of phosphotyrosine, for example, mono- or difluorophosphonomethylphenlalanine (FPmp or F₂Pmp, respectively).

In another preferred embodiment, the LRb amino acid sequence is altered by a deletion. Preferably, at least 1 but not more than 20, 15, 10, 5 amino acid(s) is deleted from the LRb sequence. In a preferred embodiment, the deletion encompasses Tyr1138 of murine LRb. In another preferred embodiment, the deletion encompasses Tyr 1141 of human LRb. In yet another preferred embodiment, the deletion can encompass a corresponding tyrosine from other species, e.g., rat.

In another aspect, the invention features an LRb amino acid sequence wherein at least one amino acid has been altered such that the LRb polypeptide has an affect on reproductive function, e.g., fertility, and/or height but not on eating behavior, e.g., body weight, appetite and/or caloric intake. In one embodiment, the amino acid sequence is a murine LRb amino acid sequence, e.g., the amino acid sequence of SEQ ID NO:2, wherein the amino acid residue at position 985 has been altered, but the amino acid position 1138 has not been altered. In another embodiment, the amino acid sequence is a human LRb amino acid sequence, e.g., the amino acid sequence of SEQ ID NO:4, wherein the amino acid residue at position 986 has been altered, but the amino acid position 1141 has not been altered. In another preferred embodiment, a corresponding tyrosine in LRb from another species (e.g., rat) has been altered. The alteration in the LRb amino acid sequence can be a deletion, addition, or substitution which encompasses amino acid residue 985 of murine LRb, or amino acid residue 986 of human LRb, or a corresponding tyrosine residue in LRb from other species. In one embodiment, the alteration of the LRb amino acid sequence can be a substitution of one or more amino acids (e.g., at least one but less than 20, 15, 10, 5 amino acids). In a preferred embodiment, the substitution includes a substitution of the amino acid residue at position 985 of SEQ ID NO:2, the amino acid residue at position 986 of SEQ ID NO:4, or a corresponding tyrosine from other species. The substituted amino acid at position 985 of SEQ ID NO:2, at position 986 of SEQ ID NO:4, or a corresponding tyrosine from other species can be any amino acid other than Tyr, e.g., Ser, Asp or Glu, or can be a Tyr analog, e.g., a Tyr analog which is not phosphorylated or a Tyr analog which remains phosphorylated. Examples of Tyr analogs include phosphotyrosine and analogs of phosphotyrosine having a hydrolysis resistant phosphorous moiety, e.g. phosphonomethylphenylalanine (Pmp), or a hydrolysis resistant phosphorous moiety which is more electronegative than the phosphate group of phosphotyrosine, for example, mono- or difluorophosphonomethylphenlalanine (FPmp or F₂Pmp, respectively).

In another preferred embodiment, the LRb amino acid sequence is altered by a deletion. Preferably, at least 1 but not more than 20, 15, 10, 5 amino acid(s) is deleted from the LRb sequence. In a preferred embodiment, the deletion encompasses Tyr985 of murine LRb. In another preferred embodiment, the deletion encompasses Tyr986 of human LRb. In yet another preferred embodiment, the deletion encompasses a corresponding tyrosine residue from another species, e.g., rat.

In another aspect, the invention features an LRb amino acid sequence wherein at least two amino acid have been altered such that the LRb polypeptide has an affect on reproductive function, e.g., fertility, and on eating behavior, e.g., body weight, appetite and/or caloric intake. In one embodiment, the amino acid sequence is a murine LRb amino acid sequence, e.g., the amino acid sequence of SEQ ID NO:2, wherein the amino acid residues at position 985 and 1138 have been altered. In another embodiment, the amino acid sequence is a human LRb amino acid sequence, e.g., the amino acid sequence of SEQ ID NO:4, wherein the amino acid residues at position 986 and 1141 have been altered. In another preferred embodiment, corresponding tyrosine residues in LRb from another species (e.g., rat) have been altered. The alteration in the LRb amino acid sequence can be a deletion, addition, or substitution which encompasses amino acid residues 985 and 1138 of murine LRb, or amino acid residues 986 and 1141 of human LRb, or corresponding tyrosine residues in LRb from other species. In one embodiment, the alteration of the LRb amino acid sequence can be a substitution of one or more amino acids (e.g., at least one but less than 20, 15, 10, 5 amino acids). In a preferred embodiment, the substitution includes a substitution of the amino acid residues at position 985 and 1138 of SEQ ID NO:2, the amino acid residues at position 986 and 1141 of SEQ ID NO:4, or corresponding tyrosine residues from other species. The substituted amino acid at position 985 and/or 1138 of SEQ ID NO:2, at position 986 and/or 1141 of SEQ ID NO:4, or one or both of the corresponding tyrosine residues from other species can be any amino acid other than Tyr, e.g., Ser, Asp or Glu, or can be a Tyr analog, e.g., a Tyr analog which is not phosphorylated or a Tyr analog which remains phosphorylated. Examples of Tyr analogs include phosphotyrosine and analogs of phosphotyrosine having a hydrolysis resistant phosphorous moiety, e.g. phosphonomethylphenylalanine (Pmp), or a hydrolysis resistant phosphorous moiety which is more electronegative than the phosphate group of phosphotyrosine, for example, mono- or difluorophosphonomethylphenlalanine (FPmp or F₂Pmp, respectively).

In another preferred embodiment, the LRb amino acid sequence is altered by a deletion. Preferably, at least 1 but not more than 20, 15, 10, 5 amino acid(s) is deleted from the LRb sequence. In a preferred embodiment, the deletion encompasses Tyr985 and/or Tyr 1138 of murine LRb. In another preferred embodiment, the deletion encompasses Tyr986 and/or Tyr 1141 of human LRb. In yet another preferred embodiment, the deletion encompasses one or both of the corresponding tyrosine residues from another species, e.g., rat.

In another aspect, the invention features an isolated portion of LRb, e.g., a biologically active portion thereof. In a preferred embodiment, the isolated LRb fragment is: a portion of murine LRb and includes Tyr985 but not Tyr1138 (e.g., a portion of SEQ ID NO:2 which includes Tyr985, but not Tyr1138); a portion from murine LRb and includes Tyr1138, but not Tyr985 (e.g., a portion of SEQ ID NO:2 which includes Tyr 1138, but not Tyr985); a portion of human LRb and includes Tyr986, but not Tyr1141 (e.g., a portion of SEQ ID NO:4 which includes Tyr986, but not Tyr1141); a portion of human LRb which includes Tyr1141, but not Tyr986 (e.g., a portion of SEQ ID NO:4 which includes Tyr 1141, but not Tyr986). In a preferred embodiment, the fragment comprises at least 10, 15, 20, 25, 30 amino acid residues of LRb.

The LRb fragments can be used to prepare anti-LRb antibodies, e.g., anti-LRb antibodies capable of binding Tyr985 but not Tyr1138 of murine LRb, anti-LRb antibodies capable of binding Tyr986 of human LRb but not Tyr1141 of human LRb, anti-LRb antibodies capable of binding Tyr1138 but not Tyr985 of murine LRb or anti-LRb antibodies capable of binding Tyr1141 but not Tyr986 of human LRb. Accordingly, the invention also provides an antigenic peptide of LRb which includes at least 8 amino acid residues of the amino acid sequence shown in SEQ ID NO:2 or SEQ ID NO:4. Preferably, the antigenic peptide includes at least 10 amino acid residues, more preferably at least 15 amino acid residues, even more preferably at least 20 amino acid residues, and most preferably at least 30, 50, 70, 80 amino acid residues. The invention further provides an antibody, e.g., a monoclonal antibody that specifically binds LRb, e.g., anti-LRb antibodies capable of binding Tyr985 but not Tyr1138 of murine LRb, anti-LRb antibodies capable of binding Tyr986 of human LRb but not Tyr1141 of human LRb, anti-LRb antibodies capable of binding Tyr1138 but not Tyr985 of murine LRb or anti-LRb antibodies capable of binding Tyr1141 but not Tyr986 of human LRb. In one embodiment, the antibody is monoclonal. In another embodiment, the antibody is coupled to a detectable substance. In yet another embodiment, the antibody is incorporated into a pharmaceutical composition comprising the antibody and a pharmaceutically acceptable carrier.

In another aspect, the invention features a nucleic acid encoding any of the LRb proteins or fragments thereof described herein.

In another aspect, the invention features a method of evaluating a sample to identify nucleic acids upregulated in one LRb pathway but not the other, e.g., upregulated in the LRb-STAT3 pathway but not the LRb-ERK pathway, or visa versa. The method includes providing a sample expression profile and at least one reference expression profile; and comparing the sample expression profile to at least one reference expression profile to thereby evaluate the sample. For example, the LRb-STAT3 pathway can be activated without activating the LRb-ERK pathway, and gene expression can be analyzed.

In a preferred embodiment, an expression profile includes a plurality of values, wherein each value corresponds to the level of expression of a different nucleic acid, splice-variant or allelic variant of a nucleic acid or a translation product thereof. The value can be a qualitative or quantitative assessment of the level of expression of the nucleic acid or the translation product of the nucleic acid, i.e., an assessment of the abundance of 1) an mRNA transcribed from the nucleic acid, or of 2) the polypeptide encoded by the nucleic acid.

In a preferred embodiment, the sample expression profile and the reference profile have a plurality of values.

In a preferred embodiment, a plurality of reference profiles is provided. A reference profile can be a profile obtained from a normal sample or a diseased sample. Preferably, the reference profile can be a population of nucleic acids, e.g., cDNA or RNA, from an animal, cell or population of cells described herein. For example, the reference can include a nucleic acid encoding an LRb having a mutation at Tyr985 (or a corresponding tyrosine) but not at Tyr1138 (or a corresponding tyrosine), a nucleic acid encoding an LRb having a mutation at Tyr1138 (or a corresponding tyrosine) but not at Tyr985 (or a corresponding tyrosine), or can have a mutation at both. In other aspects the reference profile can be from an animal, cell, or population of cells treated with an agent described herein.

In one preferred embodiment, the sample expression profile is compared to a reference profile to produce a difference profile. In a preferred embodiment, the sample expression profile is compared indirectly to the reference profile. For example, the sample expression profile is compared in multi-dimensional space to a cluster of reference profiles.

In a preferred embodiment, the sample expression profile is obtained from an array. For example, the method further includes providing an array as described above; contacting the array with a nucleic acid mixture (e.g., a mixture of nucleic acids obtained or amplified from a cell), and detecting binding of the nucleic acid mixture to the array to produce a sample expression profile. In another embodiment, the sample expression profile is determined using a method and/or apparatus that does not require an array (e.g., SAGE or quantitative PCR with multiple primers)

The method can further include harvesting mRNA from the sample and reverse transcribing the mRNA to produce cDNA, e.g., labeled or unlabelled cDNA. Optionally, the cDNA can be amplified, e.g., by a thermal cycling (e.g., polymerase chain reaction (PCR)) or an isothermal reaction (e.g., NASBA) to produce amplified nucleic acid for use as the nucleic acid mixture that is contacted to the array.

Also within the scope of the invention are agents identified using the methods described herein. The invention features a composition, e.g., a pharmaceutical composition, which includes an agent as identified and/or described herein, and a pharmaceutically acceptable carrier. The compositions of the invention can be administered alone, or in conjunction with other agents that are useful in regulating eating behavior and/or fertility.

The terms “peptides”, “proteins”, and “polypeptides” are used interchangeably herein.

The term “small molecule”, as used herein, includes peptides, peptidomimetics, or non-peptidic compounds, such as organic molecules, having a molecular weight less than 2000, preferably less than 1000.

The term “corresponding tyrosine” or “corresponding serine” as used herein refers to a tyrosine or serine residue at corresponding amino acid positions are compared. The sequences are aligned for optimal comparison purposes (e.g., gaps can be introduced in the sequence of one protein for optimal alignment with the other protein). When a position in one sequence (e.g., residue 1138 of SEQ ID NO:2) is occupied by the same amino acid residue as the corresponding position in the other sequence (e.g., residue 1141 of SEQ ID NO:4), then the amino acid residue of the second sequence is said to correspond to the position of the amino acid in the first sequence. The comparison of sequences and determination of a corresponding amino acid can be accomplished by alignment of the sequences using a mathematical algorithm. A preferred, non-limiting example of a mathematical algorithm utilized for the comparison of sequences is the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST and XBLAST programs (version 2.0) of Altschul, et al. J. Mol. Biol. 215:403-10, 1990. BLAST protein searches can be performed with the XBLAST program, score=50, wordlength=3 to compare two sequences. To obtain gapped alignments for comparison purposes. Gapped BLAST can be utilized as described in Altschul et al., Nucleic Acids Res. 25(17):3389-3402, 1997. When utilizing BLAST and Gapped BLAST programs, the default parameters of the XBLAST program can be used. See http://www.ncbi.nlm.nih.gov. Another non-limiting example of a mathematical algorithm utilized for the comparison of sequences is the algorithm of Myers and Miller, CABIOS (1989). Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. As used herein, the term “nucleic acid molecule” includes DNA molecules (e.g., a cDNA or genomic DNA) and RNA molecules (e.g., an mRNA) and analogs of the DNA or RNA generated, e.g., by the use of nucleotide analogs. The nucleic acid molecule can be single-stranded or double-stranded, but preferably is double-stranded DNA.

The term “isolated or purified nucleic acid molecule” includes nucleic acid molecules which are separated from other nucleic acid molecules which are present in the natural source of the nucleic acid. For example, with regards to genomic DNA, the term “isolated” includes nucleic acid molecules which are separated from the chromosome with which the genomic DNA is naturally associated. Preferably, an “isolated” nucleic acid is free of sequences which naturally flank the nucleic acid (i.e., sequences located at the 5′ and/or 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb or 0.1 kb of 5′ and/or 3′ nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material, or culture medium when produced by recombinant techniques, or substantially free of chemical precursors or other chemicals when chemically synthesized.

### DESCRIPTION OF THE FIGURES

FIG. 1. Generation of mice expressing LRbS1138. a. Cartoon of gene targeting strategy for the LRb-specific exon 18b. Shown in the mutant exon 18b-S1138 is the substitution mutation at Tyr1138 that also generates a novel Sph I site for genotyping. Correct recombination (dotted lines) delivers exon 18b-S 1138 and the Neo cassette into the 3′-end of the lepr locus (Genbank #AF098792), replacing wild type exon 18b while deleting the HSV-TK cassette. Also shown are the PCR primers used for tracking the mutation (arrows). b. Genotyping of knock-in mice. Tail snip genomic DNA from progeny of s/+×s/+ matings was PCR amplified using the primers shown in (A) and digested with Sph I to resolve wild-type and S1138 alleles of lepr. c. Activation of signals by leptin in the hypothalami of +/+ and s/s mice. Eight week old +/+ or s/s mice (pair-fed to +/+ animals to reduce leptin levels) were fasted for 48 hours before treatment with vehicle (−) or leptin (+) (Sigma, 100 mg/mouse IP) for 15 minutes. Mice were sacrificed and hypothalami were isolated and lysed as described 12. Lysates were immunoprecipitated with aSTAT3 (Santa Cruz) and immunoblotted with aphospho-STAT3(PY705) (BD-Pharmingen) (top panel) or directly resolved for immunoblotting with aphospho-ERK(PT/PY) (Cell Signaling) (bottom panel). d. Distribution of LRb and LRbS1138 expression. Fixed brain sections from +/+ and s/s mice were hybridized with LRb-specific probes as described in methods and exposed to a phosphorimager. LRb probe hybridization in the basomedial hypothalamus is indicated (arrows).

FIG. 2. Increased body weight of mice expressing LRbS1138. a. Obese phenotype of s/s mice. Shown is a photograph of littermate +/+ and s/s mice at 10 weeks of age. b,c. Serial body weight measurements for male (b) and female (c) mice with lepr mutations. Weights of C57BL/6Jx129Sv (solid lines)+/+ (filled circles), s/+ (filled triangles), and s/s (filled squares) mice, and C57BL/6J (dotted lines)+/+ (empty circles), db/+ (empty triangles), and db/db (empty squares) mice. Wild-type, heterozygotes, and homozygotes on each background different by Student's unpaired t-test (p<0.01) at all ages shown.

FIG. 3. Regulation of hypothalamic neuropeptide mRNA levels in the control of physiology by LRb. a-c. Levels of hypothalamic neuropeptide mRNA from 8 week old +/+, s/s, db/+, and db/db mice. Hypothalamic RNA was extracted from ad libidum-fed mice of the indicated genotypes and analyzed for relative content of POMC (a), NPY (b), and AgRP (c) mRNA by automated fluorescent RT-PCR. *p<0.01 versus control by ANOVA; NS=not significant versus control; n>6 for all genotypes tested. +/+ and s/s mice were analyzed on the same plate, as were db/+ and db/db animals. +/+ and db/+values were standardized to 100% for comparison with s/s and db/db, respectively. D. Model of LRb signals in the control of arcuate nucleus neuropeptide levels and physiology. Leptin increases POMC production in the LRb/POMC arcuate neuron via STAT3, generating a positive anorectic signal via aMSH and the melanocortin MC3 and MC4 receptors. In the LRb/NPY/AgRP neuron, leptin inhibits AgRP production via the LRb-STAT3 pathway, disinhibiting melanocortin signaling. Elevated NPY inhibits reproductive function and also blocks a component of the anorectic function of leptin directly and/or via cross-talk to the melanocortin system 20. The inhibition of NPY by LRb proceeds independently of STAT3 signaling.

FIG. 4 depicts the amino acid (SEQ ID NO:2) and the nucleic acid sequence (SEQ ID NO:1) encoding murine LRb.

FIG. 5 depicts the amino acid (SEQ ID NO:4) and the nucleic acid sequence (SEQ ID NO:3) encoding human LRb.

### DETAILED DESCRIPTION OF THE INVENTION

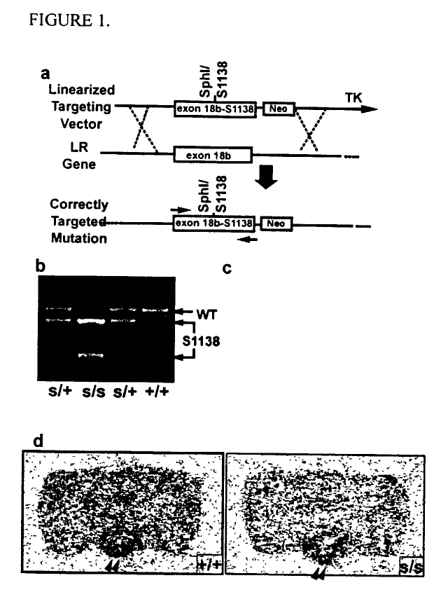

The inventors have created a transgenic mammal in which the gene for the leptin receptor has been replaced with an allele containing a substitution mutant of Tyr¹¹³⁸ (lepr^{S1138}) by homologous targeting. The transgenic mice (s/s) express LRb^{S1138} in a manner identical to wild-type LRb (FIG. 1A). LRb^{S1138} fails to activate STAT3 but mediates other signals normally⁷.

The signaling phenotype of LRbS1138 in the hypothalamus was confirmed.^{11,12} Leptin activated both STAT3 and ERK in wild-type mice, while only ERK was activated in s/s mice (FIG. 1C). Similar levels of LRb and LRb^{S1138} mRNA expression were detected by semi-quantitative RT-PCR of hypothalamic mRNA using exon-spanning primers. The pattern of LRb expression was analyzed by in situ hybridization histochemistry (FIG. 1D), which demonstrated similar LRb hybridization in the mediobasal hypothalami of +/+ and s/s mice.

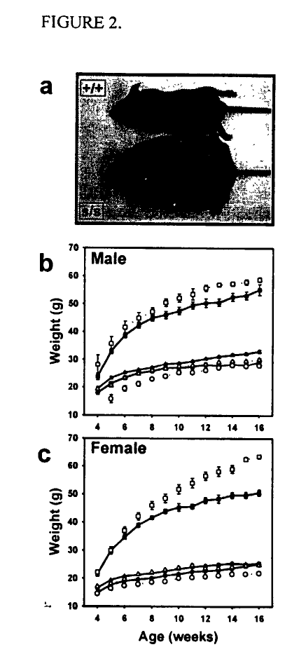

Upon visual inspection s/s mice, like db/db mice, were morbidly obese from an early age (FIG. 2A). The weight of s/s male mice (2-3 times wild-type) was similar to that of db/db mice (FIG. 2B). Furthermore, s/+ mice, like db/+ mice, were approximately 10% heavier than wild-type mice. The increased body weight of s/+ and s/s animals, as for db/+ and db/db mice, correlated with elevated leptin and whole-body triglyceride levels and thus reflected increased adiposity (Table 1). Also as in db/db mice, the obesity of s/s mice was the combined result of increased feeding (Table 1) and decreased energy expenditure, as s/s mice pair-fed to wild type mice gained weight and adipose mass more quickly than their wild type controls (data not shown). Commensurate with their level of obesity, s/s and db/db mice were hyperinsulinemic and hyperglycemic (Table 1). These results suggest that the LRb-STAT3 signal is central to the control of body energy homeostasis (the regulation of food intake and metabolic rate) by leptin. Furthermore, the amplitude of the LRb-STAT3 signal seems critical to this process, as s/+ heterozygotes consume more food and store more fat than wild-type mice do.

TABLE 1
Phenotypic data for mice carrying leprs1138 or leprdb.

| A. | | | | | | |
|---|---|---|---|---|---|---|
| Metabolic/Endocrine | +/+ | s/+ | s/s | +/+ | db/+ | db/db |
| Weight (g) | 25 8+/−30 | 27.1+/−2.5* | 45+/−5.0** | 22.9+/−1.3 | 26.0+/−1.9* | 47.2+/−2.7**,⁺⁺ |
| Leptin (ng/ml) | 3.4+/−1.1 | 3.8+/−1.1 | 55.5+/− | (ND) | 3.5+/−1.4 | 67.7+/−3.5++ |
| % Triglyceride | 6.9+/−1.1 | 12.0 +/−0.5** | 48.0+/− | (ND) | 14.6+/−0.8 | 58.6+/−1.4**,⁺⁺ |
| Feeding (g/animal/18 | 3.8+/−0.2 | 4.2+/−0.9 | 6.1+/−0.3**,⁺⁺ | (ND) | 4.1+/−0.4 | 7.6+/−0.9⁺⁺ |
| hr) | | | | | | |
| Insulin(pg/ml) | 501+/−114 | 688+/−107 | 6020+/− | 488+/−118 | 785+/−99 | 8103+/− |
| | | | 1095**,⁺⁺ | | | 1316**,⁺⁺ |
| Glucose (mM) | 7.4+/−0.4 | 8.1+/−0.7 | 15.1+/−2.2*,⁺ | 7.7+/−2.1 | 10.6+/−1.4 | 31.2+/−1.2** |
| Corticosterone (ng/dl) | 73.7+/−10.6 | 109+/−38.4 | 243+/−42.7*,+ | (ND) | 37.4+/−4.5 | 77.0+/−10.9⁺ |
| B. Reproductive | | | | | | |
| Reproduction (F) | 10/10 | 10/10 | 8/11 | 10/10 | 10/10 | 0/5 |
| Lactation (F) | + | + | − | + | + | (ND) |
| Median Onset of | 36 | (ND) | 42 | 36 | (ND) | Never (>90) |
| Estrous (days) | | | | | | |
| Corpora Lutea/Ovary | 3.6+/−0.4 | 4.0+/−0.2 | 1.9+/−0.3*,⁺ | (ND) | 3.5+/−0.4 | 0.1+/−0.1++ |
| (8 weeks) | | | | | | |
| Corpora Lutea/Ovary | 3.8+/−0.5 | 3.8+/−0.5 | 4.0+/−0.5 | (ND) | 4.2+/−0.4 | 0.1+/−0.1++ |
| (20 weeks) | | | | | | |

Table 1: Male mice of the indicated genotype were weighed and sacrificed between 1000-1200 hours during their eighth week of life. Blood was collected for glucose measurements using a glucometer and for serum determination of insulin (ELISA, Crystal Chem), leptin (ELISA, Crystal Chem), and corticosterone (RIA, ICN). The gastrointestinal tract was removed and carcasses were saponified for determination of triglyceride content to determine the percent body triglyceride of each animal 30. Food consumption was determined by weighing before the dark cycle and again eighteen hours later. All data are reported as mean +/− SEM. B. Reproductive function. Eight-week-old female mice of the indicated genotype were housed with eight-week-old male +/+ mice (1 pair/cage). The delivery of pups within six weeks was scored as reproductive success and is expressed as successfully fertile animals/animals assayed of each genotype. Lactation was determined by the ability of postpartum females to normally suckle pups to weaning and is reported as positive (+) or negative (−) for all fertile females of each genotype. Estrous cycling was determined by following vaginal smears from animals of each genotype (n>10) from the time of weaning and is reported as the median first day of fully estrogenized vaginal cells for mice of each genotype. Ovaries from 8 or 20 week old female mice (n=5 mice of each genotype) were fixed and stained with hematoxylin and eosin and scored for the number of ovulated follicles (corpora lutea) in each ovary. Mean number +/− SEM is reported. Student's unpaired t-test: *p<0.05 versus wild-type; ⁺p<0.05 versus heterozygote; **p<0.001 versus wild-type; ⁺⁺p<0.001 versus heterozygote.

As well as regulating satiety and metabolic function, leptin plays a permissive role in neuroendocrine function-signaling sufficiency of energy stores to block the endocrine starvation response¹³. Leptin inhibits adrenal cortical function and db/db mice display increased levels of corticosterone 14; similarly, adrenal corticosteroid levels were increased in s/s mice (Table 1). The control of adrenal cortical function by leptin thus also depends upon the integrity of the LRb-STAT3 signal.

However, while s/+ mice were heavier than db/+ mice (as well as wild-type mice), s/s males were approximately 5% lighter than db/db males and trended toward a less severe metabolic phenotype than that displayed by db/db mice (FIG. 2B, Table 1). Indeed, this disparity was more pronounced in females, where s/s animals were approximately 20% lighter than db/db mice of a comparable age (FIG. 2C). While not wanting to be bound by theory, the differences in energy homeostasis between s/s and db/db mice might be attributable to genetic background, or leptin-regulated pathways might not be universally disrupted in s/s mice.

Defects in leptin action impair fertility by interfering with the hypothalamic control of gonad function across genotypes and species—especially among females¹⁵⁻¹⁷. Controlled breeding studies with wild-type mates demonstrated that most female s/s mice produced progeny that survived to birth (Table 1), while db/db females failed to reproduce. Fertile s/s females failed to lactate normally post-partum, however, suggesting a role for LRb-STAT3 signaling in the control of lactation independent of fertility. In similar experiments, the majority of male s/s mice were also fertile (not shown). We quantified fertility in s/s females by examining the onset of estrous cycling and gonad histology (Table 1). While female db/db animals never commenced estrous cycles in the period observed, the onset of vaginal estrous was only slightly delayed in s/s females compared to wild type mice (Table 1). Similarly, the reproductive tracts of db/db females were small and atrophic, while the reproductive tracts of s/s females appeared grossly normal (not shown). Histologically, all s/s ovaries demonstrated evidence of ovulation, and although fewer corpora lutea were present in s/s ovaries compared with wild type ovaries at 8 weeks, numbers of corpora lutea in s/s ovaries normalized by 20 weeks. No evidence of ovulation was observed in db/db ovaries at either age. Thus, in contrast to the absence of gonad function in db/db females, gonad function in s/s females is essentially normal although slightly delayed. These results suggest that the hypothalamic control of reproduction by leptin is predominantly regulated independently of LRb-STAT3 signaling and depends on other LRb-mediated signals that are unperturbed in LRb^{S1138}. The lag in the onset of full ovarian function observed in s/s females may reflect a primary reproductive defect (i.e. a minor direct role for LRb-STAT3 signaling in reproductive control) or may be secondary to the abnormal metabolic and hormonal milieu in the s/s animals.

The similarity of the metabolic phenotypes and discordance of the reproductive phenotypes of s/s and db/db mice suggests that the LRb-STAT3 signal is required only for a subset of hypothalamic leptin responses. Two distinct populations of LRb-expressing neurons in the arcuate nucleus of the hypothalamus are thought to importantly effect leptin action^{18,19}. One population co-expresses LRb and proopiomelanocortin (POMC), the precursor molecule for αMSH, which mediates signals via the hypothalamic MC3 and MC4 melanocortin receptors^{18,20}. Leptin is thought to activate these LRb/POMC neurons, and this melanocortin action promotes anorexia. The second population of LRb-expressing arcuate neurons, co-expresses the appetite-enhancing (orexigenic) peptides neuropeptide Y (NPY) and agouti-related protein (AgRP, a MC3/MC4 receptor antagonist). This LRb/NPY/AgRP neuron is thought to be hyperpolarized and inhibited by leptin^{18,21,22}. In addition to stimulating feeding, NPY plays a role in the hypothalamic control of reproductive function: Chronically high levels of NPY block reproductive function and increased NPY action mediates some component of infertility in animals defective in leptin action²³⁻²⁵. One possible explanation for the split (metabolic versus reproductive) phenotype of s/s mice, therefore, is that the LRb-STAT3 signal might be critical to melanocortin function, but not involved in the regulation of NPY in the hypothalamus. Indeed, ob/ob NPY^{-/-} mice, like s/s mice, display moderately decreased obesity compared to ob/ob mice on a C57BL/6Jx129Sv background (with more dramatic differences observed in females than in males) 23. Furthermore, while ob/ob mice are infertile and groom poorly on this background, removal of NPY in ob/ob NPY−/− mice restores grooming and partially restores reproductive function; s/s mice groom normally as well as being fertile.

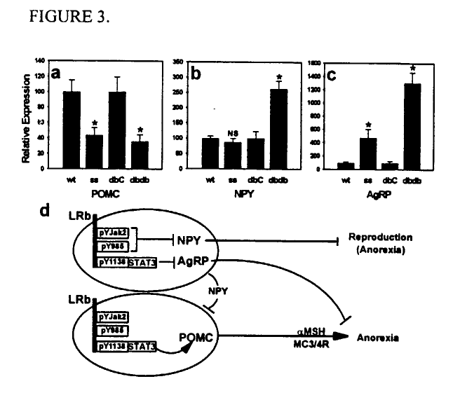

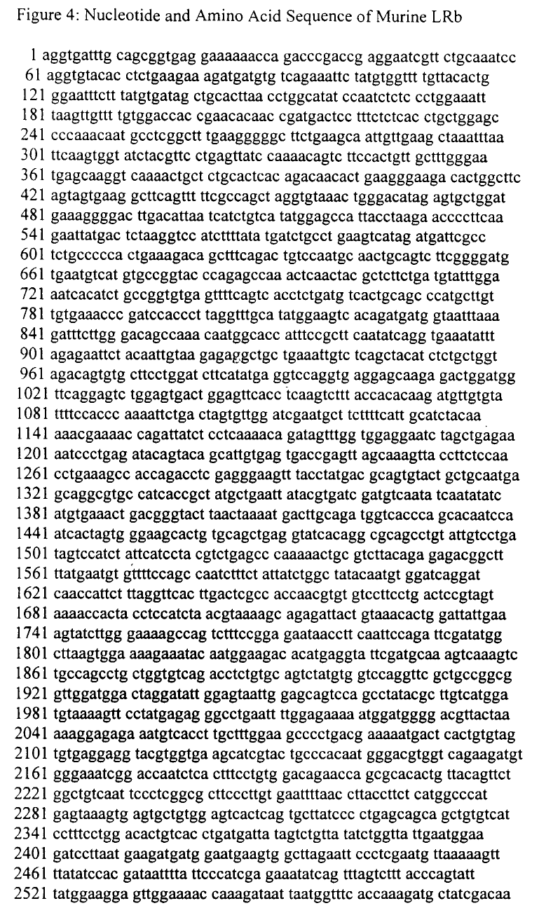

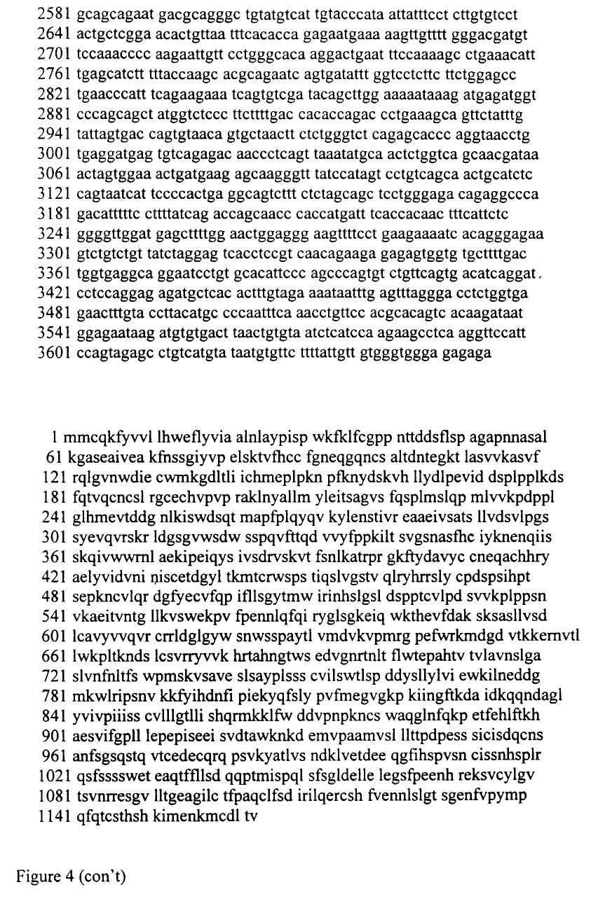

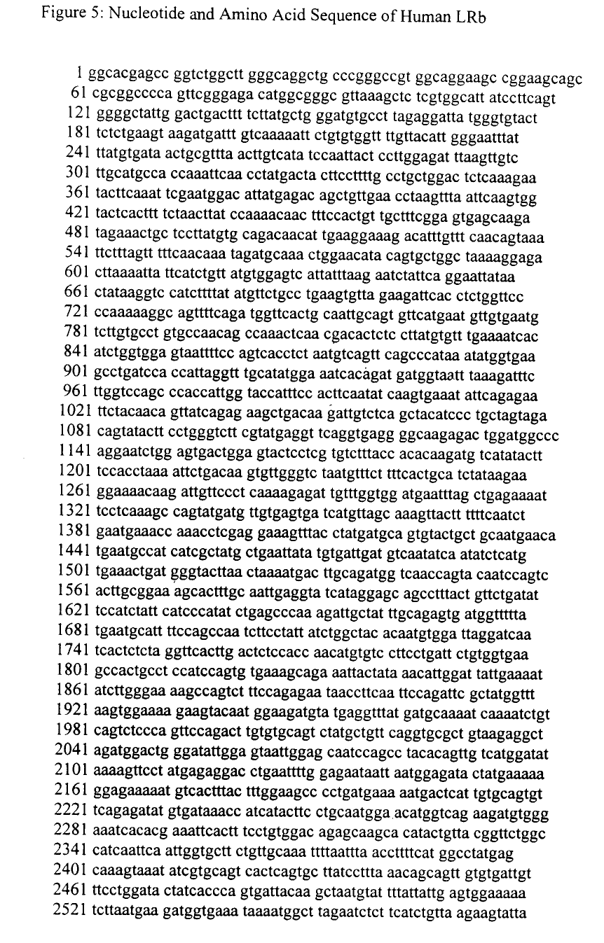

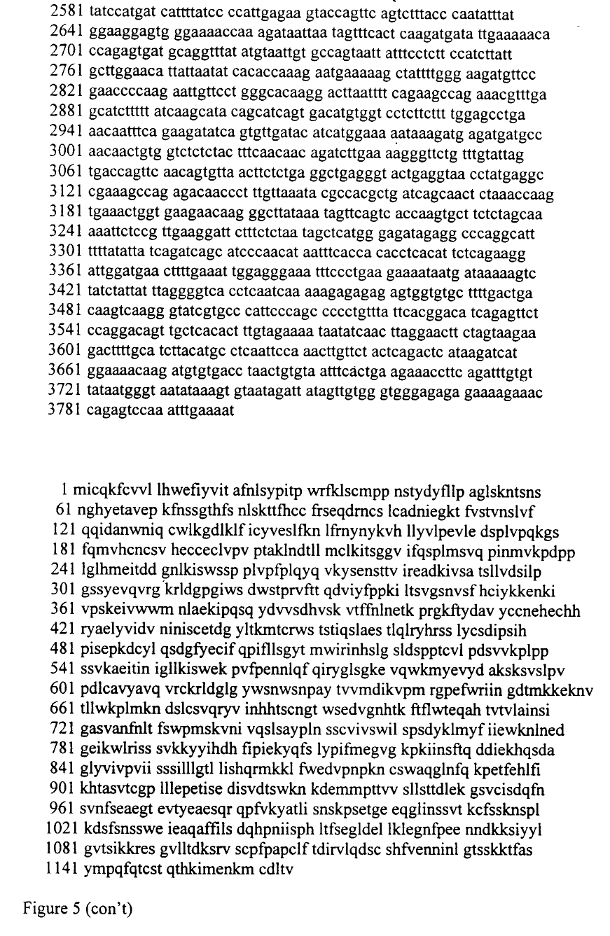

In order to test this model, we measured hypothalamic neuropeptide levels in s/s and db/db mice by RT-PCR (FIG. 3A-C). The expression of hypothalamic POMC mRNA was impaired in s/s mice as in db/db mice, confirming that LRb (similar to other cytokine receptors²⁶) regulates POMC levels via STAT3. In contrast, disruption of the LRb-STAT3 signal did not alter hypothalamic NPY mRNA levels, which were increased (as expected) in db/db mice, but slightly (although not significantly) decreased in s/s mice. AgRP mRNA levels were also increased in the hypothalami of s/s mice (as in db/db mice), suggesting that the LRb-STAT3 signal mediates repression of AgRP transcription. Thus, the function of the LRb-STAT3 transcriptional signal is important for the regulation of melanocortin signaling in both the LRb/POMC neuron and (via AgRP) in the LRb/NPY/AgRP neuron, but other LRb-mediated signals control non-melanocortin function (such as NPY regulation) in the LRb/NPY/AgRP neuron. The LRb-STAT3 signal is thus specific for the physiological endpoint, not the cell type, as it distinguishes the neural processes responsible for the control of appetite (primarily melanocortins) and fertility (primarily NPY). The presence of other neurotransmitters in the arcuate NPY/AgRP/LRb co-expressing neurons may explain the increased fertility of our mice compared to ob/ob NPY^{-/-} mice, as the regulation and release of these transmitters by leptin is likely to be dysregulated in ob/ob NPY^{-/-}, animals, but may be normal in s/s mice. The control of membrane potential by leptin in arcuate LRb neurons is rapid, suggesting that it is unlikely to be regulated downstream of new mRNA transcription (as by STAT3); indeed, PI 3′-kinase appears to regulate the control of membrane potential by leptin^{20,21,27}.

The results described herein confirm that the LRb-STAT3 pathway is an important regulator of some aspects of physiology by leptin, but also demonstrate that this signal controls only a subset of physiologic leptin functions by regulating specific neuronal processes. Not only do these results indicate that the LRb-STAT3 pathway represents a site of leptin resistance and potential therapeutic target in obesity, they more generally suggest that individual signals in other receptor systems may mediate distinguishable aspects of mammalian physiology.

Generation of Analogs: Production of Altered DNA and Peptide Sequences by Random Methods

Amino acid sequence variants of a protein, e.g., a LRb or STAT3 or ERK, can be prepared by random mutagenesis of DNA which encodes a protein or a particular domain or region of a protein. Useful methods include PCR mutagenesis and saturation mutagenesis. A library of random amino acid sequence variants can also be generated by the synthesis of a set of degenerate oligonucleotide sequences. (Methods for screening proteins in a library of variants, e.g., screening for LRb or STAT3 or ERK modulating activity, are elsewhere herein.)

PCR Mutagenesis

In PCR mutagenesis, reduced Taq polymerase fidelity is used to introduce random mutations into a cloned fragment of DNA (Leung et al., 1989, Technique 1:11-15). This is a very powerful and relatively rapid method of introducing random mutations. The DNA region to be mutagenized is amplified using the polymerase chain reaction (PCR) under conditions that reduce the fidelity of DNA synthesis by Taq DNA polymerase, e.g., by using a dGTP/dATP ratio of five and adding Mn²⁺ to the PCR reaction. The pool of amplified DNA fragments are inserted into appropriate cloning vectors to provide random mutant libraries.

Saturation Mutagenesis

Saturation mutagenesis allows for the rapid introduction of a large number of single base substitutions into cloned DNA fragments (Mayers et al., 1985, Science 229:242). This technique includes generation of mutations, e.g., by chemical treatment or irradiation of single-stranded DNA in vitro, and synthesis of a complimentary DNA strand. The mutation frequency can be modulated by modulating the severity of the treatment, and essentially all possible base substitutions can be obtained. Because this procedure does not involve a genetic selection for mutant fragments both neutral substitutions, as well as those that alter function, are obtained. The distribution of point mutations is not biased toward conserved sequence elements.

Degenerate Oligonucleotides

A library of homologs can also be generated from a set of degenerate oligonucleotide sequences. Chemical synthesis of a degenerate sequences can be carried out in an automatic DNA synthesizer, and the synthetic genes then ligated into an appropriate expression vector. The synthesis of degenerate oligonucleotides is known in the art (see for example, Narang, S A (1983) Tetrahedron 39:3; Itakura et al. (1981) Recombinant DNA, Proc 3rd Cleveland Sympos. Macromolecules, ed. A G Walton, Amsterdam: Elsevier pp273-289; Itakura et al. (1984) Annu. Rev. Biochem. 53:323; Itakura et al. (1984) Science 198:1056; Ike et al. (1983) Nucleic Acid Res. 11:477. Such techniques have been employed in the directed evolution of other proteins (see, for example, Scott et al. (1990) Science 249:386-390; Roberts et al. (1992) PNAS 89:2429-2433; Devlin et al. (1990) Science 249: 404-406; Cwirla et al. (1990) PNAS 87: 6378-6382; as well as U.S. Pat. Nos. 5,223,409, 5,198,346, and 5,096,815).

Generation of Analogs: Production of Altered DNA and Peptide Sequences by Directed Mutagenesis

Non-random or directed, mutagenesis techniques can be used to provide specific sequences or mutations in specific regions. These techniques can be used to create variants which include, e.g., deletions, insertions, or substitutions, of residues of the known amino acid sequence of a protein. The sites for mutation can be modified individually or in series, e.g., by (1) substituting first with conserved amino acids and then with more radical choices depending upon results achieved, (2) deleting the target residue, or (3) inserting residues of the same or a different class adjacent to the located site, or combinations of options 1-3.

Alanine Scanning Mutagenesis

Alanine scanning mutagenesis is a useful method for identification of certain residues or regions of the desired protein that are preferred locations or domains for mutagenesis, Cunningham and Wells (Science 244:1081-1085, 1989). In alanine scanning, a residue or group of target residues are identified (e.g., charged residues such as Arg, Asp, His, Lys, and Glu) and replaced by a neutral or negatively charged amino acid (most preferably alanine or polyalanine). Replacement of an amino acid can affect the interaction of the amino acids with the surrounding aqueous environment in or outside the cell. Those domains demonstrating functional sensitivity to the substitutions are then refined by introducing further or other variants at or for the sites of substitution. Thus, while the site for introducing an amino acid sequence variation is predetermined, the nature of the mutation per se need not be predetermined. For example, to optimize the performance of a mutation at a given site, alanine scanning or random mutagenesis may be conducted at the target codon or region and the expressed desired protein subunit variants are screened for the optimal combination of desired activity.

Oligonucleotide-Mediated Mutagenesis

Oligonucleotide-mediated mutagenesis is a useful method for preparing substitution, deletion, and insertion variants of DNA, see, e.g., Adelman et al., (DNA 2:183, 1983). Briefly, the desired DNA is altered by hybridizing an oligonucleotide encoding a mutation to a DNA template, where the template is the single-stranded form of a plasmid or bacteriophage containing the unaltered or native DNA sequence of the desired protein. After hybridization, a DNA polymerase is used to synthesize an entire second complementary strand of the template that will thus incorporate the oligonucleotide primer, and will code for the selected alteration in the desired protein DNA. Generally, oligonucleotides of at least 25 nucleotides in length are used. An optimal oligonucleotide will have 12 to 15 nucleotides that are completely complementary to the template on either side of the nucleotide(s) coding for the mutation. This ensures that the oligonucleotide will hybridize properly to the single-stranded DNA template molecule. The oligonucleotides are readily synthesized using techniques known in the art such as that described by Crea et al. (Proc. Natl. Acad. Sci. (1978) USA, 75: 5765).

Cassette Mutagenesis

Another method for preparing variants, cassette mutagenesis, is based on the technique described by Wells et al. (Gene, 34:315[1985]). The starting material is a plasmid (or other vector) which includes the protein subunit DNA to be mutated. The codon(s) in the protein subunit DNA to be mutated are identified. There must be a unique restriction endonuclease site on each side of the identified mutation site(s). If no such restriction sites exist, they may be generated using the above-described oligonucleotide-mediated mutagenesis method to introduce them at appropriate locations in the desired protein subunit DNA. After the restriction sites have been introduced into the plasmid, the plasmid is cut at these sites to linearize it. A double-stranded oligonucleotide encoding the sequence of the DNA between the restriction sites but containing the desired mutation(s) is synthesized using standard procedures. The two strands are synthesized separately and then hybridized together using standard techniques. This double-stranded oligonucleotide is referred to as the cassette. This cassette is designed to have 3′ and 5′ ends that are comparable with the ends of the linearized plasmid, such that it can be directly ligated to the plasmid. This plasmid now contains the mutated desired protein subunit DNA sequence.

Combinatorial Mutagenesis

Combinatorial mutagenesis can also be used to generate mutants. For example, the amino acid sequences for a group of homologs or other related proteins are aligned, preferably to promote the highest homology possible. All of the amino acids which appear at a given position of the aligned sequences can be selected to create a degenerate set of combinatorial sequences. The variegated library of variants is generated by combinatorial mutagenesis at the nucleic acid level, and is encoded by a variegated gene library. For example, a mixture of synthetic oligonucleotides can be enzymatically ligated into gene sequences such that the degenerate set of potential sequences are expressible as individual peptides, or alternatively, as a set of larger fusion proteins containing the set of degenerate sequences.

Primary High-Through-Put Methods for Screening Libraries of Peptide Fragments or Homologs

Various techniques are known in the art for screening generated mutant gene products. Techniques for screening large gene libraries often include cloning the gene library into replicable expression vectors, transforming appropriate cells with the resulting library of vectors, and expressing the genes under conditions in which detection of a desired activity, assembly into a trimeric molecules, binding to natural ligands, e.g., a receptor or substrates, facilitates relatively easy isolation of the vector encoding the gene whose product was detected. Each of the techniques described below is amenable to high through-put analysis for screening large numbers of sequences created, e.g., by random mutagenesis techniques.

A high throughput assay for inhibition of STAT3 signaling has been described, e.g., by Robert Fenton, R21 National Cancer Institute Grant No. CA91136, available from the NIH-NCI web site.

Two Hybrid Systems

Two hybrid (interaction trap) assays can be used to identify a protein that interacts with a LRb-STAT3 signaling molecule, e.g., LRb or STAT3 (or an LRb-ERK signaling molecule). These may include, e.g., agonists, superagonists, and antagonists of LRb or STAT3. (The subject protein and a protein it interacts with are used as the bait protein and fish proteins.). These assays rely on detecting the reconstitution of a functional transcriptional activator mediated by protein-protein interactions with a bait protein. In particular, these assays make use of chimeric genes which express hybrid proteins. The first hybrid comprises a DNA-binding domain fused to the bait protein. e.g., a LRb or STAT3 molecule or a fragment thereof. The second hybrid protein contains a transcriptional activation domain fused to a “fish” protein, e.g. an expression library. If the fish and bait proteins are able to interact, they bring into close proximity the DNA-binding and transcriptional activator domains. This proximity is sufficient to cause transcription of a reporter gene which is operably linked to a transcriptional regulatory site which is recognized by the DNA binding domain, and expression of the marker gene can be detected and used to score for the interaction of the bait protein with another protein.

Although many of the methods described herein are discussed with regards to LRb-STAT3 signaling molecules, they are also applicable to LRb-ERK signaling molecules.

Display Libraries

In one approach to screening assays, the candidate peptides are displayed on the surface of a cell or viral particle, and the ability of particular cells or viral particles to bind an appropriate receptor protein via the displayed product is detected in a “panning assay”. For example, the gene library can be cloned into the gene for a surface membrane protein of a bacterial cell, and the resulting fusion protein detected by panning (Ladner et al., WO 88/06630; Fuchs et al. (1991) Bio/Technology 9:1370-1371; and Goward et al. (1992) TIBS 18:136-140). In a similar fashion, a detectably labeled ligand can be used to score for potentially functional peptide homologs. Fluorescently labeled ligands, e.g., receptors, can be used to detect homolog which retain ligand-binding activity. The use of fluorescently labeled ligands, allows cells to be visually inspected and separated under a fluorescence microscope, or, where the morphology of the cell permits, to be separated by a fluorescence-activated cell sorter.

A gene library can be expressed as a fusion protein on the surface of a viral particle. For instance, in the filamentous phage system, foreign peptide sequences can be expressed on the surface of infectious phage, thereby conferring two significant benefits. First, since these phage can be applied to affinity matrices at concentrations well over 10¹³ phage per milliliter, a large number of phage can be screened at one time. Second, since each infectious phage displays a gene product on its surface, if a particular phage is recovered from an affinity matrix in low yield, the phage can be amplified by another round of infection. The group of almost identical E. coli filamentous phages M13, fd., and fl are most often used in phage display libraries. Either of the phage gIII or gVIII coat proteins can be used to generate fusion proteins without disrupting the ultimate packaging of the viral particle. Foreign epitopes can be expressed at the NH₂-terminal end of pIII and phage bearing such epitopes recovered from a large excess of phage lacking this epitope (Ladner et al. PCT publication WO 90/02909; Garrard et al., PCT publication WO 92/09690; Marks et al. (1992) J Biol. Chem. 267:16007-16010; Griffiths et al. (1993) EMBO J 12:725-734; Clackson et al. (1991) Nature 352:624-628; and Barbas et al. (1992) PNAS 89:4457-4461).

A common approach uses the maltose receptor of E. coli (the outer membrane protein, LamB) as a peptide fusion partner (Charbit et al. (1986) EMBO 5, 3029-3037). Oligonucleotides have been inserted into plasmids encoding the LamB gene to produce peptides fused into one of the extracellular loops of the protein. These peptides are available for binding to ligands, e.g., to antibodies, and can elicit an immune response when the cells are administered to animals. Other cell surface proteins, e.g., OmpA (Schorr et al. (1991) Vaccines 91, pp. 387-392), PhoE (Agterberg, et al. (1990) Gene 88, 37-45), and PAL (Fuchs et al. (1991) Bio/Tech 9, 1369-1372), as well as large bacterial surface structures have served as vehicles for peptide display. Peptides can be fused to pilin, a protein which polymerizes to form the pilus-a conduit for interbacterial exchange of genetic information (Thiry et al. (1989) Appl. Environ. Microbiol. 55, 984-993). Because of its role in interacting with other cells, the pilus provides a useful support for the presentation of peptides to the extracellular environment. Another large surface structure used for peptide display is the bacterial motive organ, the flagellum. Fusion of peptides to the subunit protein flagellin offers a dense array of may peptides copies on the host cells (Kuwajima et al. (1988) Bio/Tech. 6, 1080-1083). Surface proteins of other bacterial species have also served as peptide fusion partners. Examples include the Staphylococcus protein A and the outer membrane protease IgA of Neisseria (Hansson et al. (1992) J. Bacteriol. 174, 4239-4245 and Klauser et al. (1990) EMBO J. 9, 1991-1999).

In the filamentous phage systems and the LamB system described above, the physical link between the peptide and its encoding DNA occurs by the containment of the DNA within a particle (cell or phage) that carries the peptide on its surface. Capturing the peptide captures the particle and the DNA within. An alternative scheme uses the DNA-binding protein LacI to form a link between peptide and DNA (Cull et al. (1992) PNAS USA 89:1865-1869). This system uses a plasmid containing the LacI gene with an oligonucleotide cloning site at its 3′-end. Under the controlled induction by arabinose, a LacI-peptide fusion protein is produced. This fusion retains the natural ability of LacI to bind to a short DNA sequence known as LacO operator (LacO). By installing two copies of LacO on the expression plasmid, the LacI-peptide fusion binds tightly to the plasmid that encoded it. Because the plasmids in each cell contain only a single oligonucleotide sequence and each cell expresses only a single peptide sequence, the peptides become specifically and stably associated with the DNA sequence that directed its synthesis. The cells of the library are gently lysed and the peptide-DNA complexes are exposed to a matrix of immobilized receptor to recover the complexes containing active peptides. The associated plasmid DNA is then reintroduced into cells for amplification and DNA sequencing to determine the identity of the peptide ligands. As a demonstration of the practical utility of the method, a large random library of dodecapeptides was made and selected on a monoclonal antibody raised against the opioid peptide dynorphin B. A cohort of peptides was recovered, all related by a consensus sequence corresponding to a six-residue portion of dynorphin B. (Cull et al. (1992) Proc. Natl. Acad. Sci. U.S.A. 89-1869)

This scheme, sometimes referred to as peptides-on-plasmids, differs in two important ways from the phage display methods. First, the peptides are attached to the C-terminus of the fusion protein, resulting in the display of the library members as peptides having free carboxy termini. Both of the filamentous phage coat proteins, pIII and pVIII, are anchored to the phage through their C-termini, and the guest peptides are placed into the outward-extending N-terminal domains. In some designs, the phage-displayed peptides are presented right at the amino terminus of the fusion protein. (Cwirla, et al. (1990) Proc. Natl. Acad. Sci. U.S.A. 87, 6378-6382) A second difference is the set of biological biases affecting the population of peptides actually present in the libraries. The LacI fusion molecules are confined to the cytoplasm of the host cells. The phage coat fusions are exposed briefly to the cytoplasm during translation but are rapidly secreted through the inner membrane into the periplasmic compartment, remaining anchored in the membrane by their C-terminal hydrophobic domains, with the N-termini, containing the peptides, protruding into the periplasm while awaiting assembly into phage particles. The peptides in the LacI and phage libraries may differ significantly as a result of their exposure to different proteolytic activities. The phage coat proteins require transport across the inner membrane and signal peptidase processing as a prelude to incorporation into phage. Certain peptides exert a deleterious effect on these processes and are underrepresented in the libraries (Gallop et al. (1994) J. Med. Chem. 37(9):1233-1251). These particular biases are not a factor in the LacI display system.

The number of small peptides available in recombinant random libraries is enormous. Libraries of 10⁷-10⁹ independent clones are routinely prepared. Libraries as large as 10¹¹ recombinants have been created, but this size approaches the practical limit for clone libraries. This limitation in library size occurs at the step of transforming the DNA containing randomized segments into the host bacterial cells. To circumvent this limitation, an in vitro system based on the display of nascent peptides in polysome complexes has recently been developed. This display library method has the potential of producing libraries 3-6 orders of magnitude larger than the currently available phage/phagemid or plasmid libraries. Furthermore, the construction of the libraries, expression of the peptides, and screening, is done in an entirely cell-free format.

In one application of this method (Gallop et al. (1994) J. Med. Chem. 37(9):1233-1251), a molecular DNA library encoding 10¹² decapeptides was constructed and the library expressed in an E. coli S30 in vitro coupled transcription/translation system. Conditions were chosen to stall the ribosomes on the mRNA, causing the accumulation of a substantial proportion of the RNA in polysomes and yielding complexes containing nascent peptides still linked to their encoding RNA. The polysomes are sufficiently robust to be affinity purified on immobilized receptors in much the same way as the more conventional recombinant peptide display libraries are screened. RNA from the bound complexes is recovered, converted to cDNA, and amplified by PCR to produce a template for the next round of synthesis and screening. The polysome display method can be coupled to the phage display system. Following several rounds of screening, cDNA from the enriched pool of polysomes was cloned into a phagemid vector. This vector serves as both a peptide expression vector, displaying peptides fused to the coat proteins, and as a DNA sequencing vector for peptide identification. By expressing the polysome-derived peptides on phage, one can either continue the affinity selection procedure in this format or assay the peptides on individual clones for binding activity in a phage ELISA, or for binding specificity in a completion phage ELISA (Barret, et al. (1992) Anal. Biochem 204,357-364). To identify the sequences of the active peptides one sequences the DNA produced by the phagemid host.

Secondary Screens

The high through-put assays described above can be followed by secondary screens in order to identify further biological activities which will, e.g., allow one skilled in the art to differentiate agonists from antagonists. The type of a secondary screen used will depend on the desired activity that needs to be tested. For example, an assay can be developed in which the ability to inhibit an interaction between a protein of interest (e.g., LRb or STAT3) and a ligand (e.g., a LRb or STAT3 substrate) can be used to identify antagonists from a group of peptide fragments isolated though one of the primary screens described above.

Therefore, methods for generating fragments and analogs and testing them for activity are known in the art. Once the core sequence of interest is identified, it is routine to perform for one skilled in the art to obtain analogs and fragments.

Although many of the methods described herein are discussed with regards to LRb-STAT3 signaling molecules, they are also applicable to LRb-ERK signaling molecules.

Nucleic Acid Arrays

Arrays are useful molecular tools for characterizing a sample by multiple criteria. For example, an array having a capture probes for one or more nucleic acids described herein can be used to assess the activation of one LRb pathway but not the other. Arrays can have many addresses, e.g., locatable sites, on a substrate. The featured arrays can be configured in a variety of formats, non-limiting examples of which are described below.

The substrate can be opaque, translucent, or transparent. The addresses can be distributed, on the substrate in one dimension, e.g., a linear array; in two dimensions, e.g., a planar array; or in three dimensions, e.g., a three dimensional array. The solid substrate may be of any convenient shape or form, e.g., square, rectangular, ovoid, or circular. Non-limiting examples of two-dimensional array substrates include glass slides, quartz (e.g., UV-transparent quartz glass), single crystal silicon, wafers (e.g., silica or plastic), mass spectroscopy plates, metal coated substrates (e.g., gold), membranes (e.g., nylon and nitrocellulose), plastics and polymers (e.g., polystyrene, polypropylene, polyvinylidene difluoride, poly-tetrafluoroethylene, polycarbonate, PDMS, nylon, acrylic, and the like). Three-dimensional array substrates include porous matrices, e.g., gels or matrices. Potentially useful porous substrates include: agarose gels, acrylamide gels, sintered glass, dextran, meshed polymers (e.g., macroporous crosslinked dextran, sephacryl, and sepharose), and so forth.

The array can have a density of at least than 10, 50, 100, 200, 500, 1 000, 2 000, 10⁴, 10⁵, 10⁶, 10⁷, 10⁸, or 10⁹ or more addresses per cm² and ranges between. In a preferred embodiment, the plurality of addresses includes at least 10, 100, 500, 1 000, 5 000, 10 000, or 50 000 addresses. In a preferred embodiment, the plurality of addresses includes less than 9, 99, 499, 999, 4 999, 9 999, or 49 999 addresses. Addresses in addition to the address of the plurality can be disposed on the array. The center to center distance can be 5 mm, 1 mm, 100 um, 10 um, 1 um or less. The longest diameter of each address can be 5 mm, 1 mm, 100 um, 10 um, 1 um or less. Each addresses can contain 0 ug, 1 ug, 100 ng, 10 ng, 1 ng, 100 pg, 10 pg, 1 pg, 0.1 pg, or less of a capture agent, i.e. the capture probe. For example, each address can contain 100, 10³, 10⁴, 10⁵, 10⁶, 10⁷, 10⁸, or 10⁹ or more molecules of the nucleic acid.

Arrays can be fabricated by a variety of methods, e.g., photolithographic methods (see, e.g., U.S. Pat. Nos. 5,143,854; 5,510,270; and. 5,527,681), mechanical methods (e.g., directed-flow methods as described in U.S. Pat. No. 5,384,261), pin based methods (e.g., as described in U.S. Pat. No. 5,288,514), and bead based techniques (e.g., as described in PCT US/93/04145). The capture probe can be a single-stranded nucleic acid, a double-stranded nucleic acid (e.g., which is denatured prior to or during hybridization), or a nucleic acid having a single-stranded region and a double-stranded region. Preferably, the capture probe is single-stranded. The capture probe can be selected by a variety of criteria, and preferably is designed by a computer program with optimization parameters. The capture probe can be selected to hybridize to a sequence rich (e.g., non-homopolymeric) region of the nucleic acid. The Tₘ of the capture probe can be optimized by prudent selection of the complementarity region and length. Ideally, the Tₘ of all capture probes on the array is similar, e.g., within 20, 10, 5, 3, or 2° C. of one another. A database scan of available sequence information for a species can be used to determine potential cross-hybridization and specificity problems.

The isolated nucleic acid is preferably mRNA that can be isolated by routine methods, e.g., including DNase treatment to remove genomic DNA and hybridization to an oligo-dT coupled solid substrate (e.g., as described in Current Protocols in Molecular Biology, John Wiley & Sons, N.Y.). The substrate is washed, and the mRNA is eluted.

The isolated mRNA can be reversed transcribed and optionally amplified, e.g., by rtPCR, e.g., as described in (U.S. Pat. No. 4,683,202). The nucleic acid can be an amplification product, e.g., from PCR (U.S. Pat. Nos. 4,683,196 and 4,683,202); rolling circle amplification (“RCA,” U.S. Pat. No. 5,714,320), isothermal RNA amplification or NASBA (U.S. Pat. Nos. 5,130,238; 5,409,818; and 5,554,517), and strand displacement amplification (U.S. Pat. No. 5,455,166). The nucleic acid can be labeled during amplification, e.g., by the incorporation of a labeled nucleotide. Examples of preferred labels include fluorescent labels, e.g., red-fluorescent dye Cy5 (Amersham) or green-fluorescent dye Cy3 (Amersham), and chemiluminescent labels, e.g., as described in U.S. Pat. No. 4,277,437. Alternatively, the nucleic acid can be labeled with biotin, and detected after hybridization with labeled streptavidin, e.g., streptavidin-phycoerythrin (Molecular Probes).

The labeled nucleic acid can be contacted to the array. In addition, a control nucleic acid or a reference nucleic acid can be contacted to the same array. The control nucleic acid or reference nucleic acid can be labeled with a label other than the sample nucleic acid, e.g., one with a different emission maximum. Labeled nucleic acids can be contacted to an array under hybridization conditions. The array can be washed, and then imaged to detect fluorescence at each address of the array.

The expression data can be stored in a database, e.g., a relational database such as a SQL database (e.g., Oracle or Sybase database environments). The database can have multiple tables. For example, raw expression data can be stored in one table, wherein each column corresponds to a nucleic acid being assayed, e.g., an address or an array, and each row corresponds to a sample. A separate table can store identifiers and sample information, e.g., the batch number of the array used, date, and other quality control information.

Nucleic acids that are similarly regulated during activation of an LRb pathway (e.g., an LRb-STAT3 pathway or an LRb-ERK pathway) can be identified by clustering expression data to identify coregulated nucleic acids. Nucleic acids can be clustered using hierarchical clustering (see, e.g., Sokal and Michener (1958) Univ. Kans. Sci. Bull. 38:1409), Bayesian clustering, k-means clustering, and self-organizing maps (see, Tamayo et al. (1999) Proc. Natl. Acad. Sci. USA 96:2907).

Expression profiles obtained from nucleic acid expression analysis on an array can be used to compare samples and/or cells in a variety of states as described in Golub et al. ((1999) Science 286:531). In one embodiment, multiple expression profiles from different conditions and including replicates or like samples from similar conditions are compared to identify nucleic acids whose expression level is predictive of the sample and/or condition. Each candidate nucleic acid can be given a weighted “voting” factor dependent on the degree of correlation of the nucleic acid's expression and the sample identity. A correlation can be measured using a Euclidean distance or the Pearson correlation coefficient.

The similarity of a sample expression profile to a predictor expression profile (e.g., a reference expression profile that has associated weighting factors for each nucleic acid) can then be determined, e.g., by comparing the log of the expression level of the sample to the log of the predictor or reference expression value and adjusting the comparison by the weighting factor for all nucleic acids of predictive value in the profile.

Polypeptide Arrays

The expression level of a polypeptide described herein can be determined using an antibody specific for the polypeptide (e.g., using a Western blot or an ELISA assay). Moreover, the expression levels of multiple polypeptides encoded by the nucleic acids described herein can be rapidly determined in parallel using a polypeptide array having antibody capture probes for each of the polypeptides. Antibodies specific for a polypeptide can be generated by a method described herein (see “Antibodies”).

A low-density (96 well format) protein array has been developed in which proteins are spotted onto a nitrocellulose membrane Ge, H. (2000) Nucleic Acids Res. 28, e3, I-VII). A high-density protein array (100,000 samples within 222×222 nm) used for antibody screening was formed by spotting proteins onto polyvinylidene difluoride (PVDF) (Lueking et al. (1999) Anal. Biochem. 270, 103-111). Polypeptides can be printed on a flat glass plate that contained wells formed by an enclosing hydrophobic Teflon mask (Mendoza, et al. (1999). Biotechniques 27, 778-788.). Also, polypeptide can be covalently linked to chemically derivatized flat glass slides in a high-density array (1600 spots per square centimeter) (MacBeath, G., and Schreiber, S. L. (2000) Science 289, 1760-1763). De Wildt et al., describe a high-density array of 18,342 bacterial clones, each expressing a different single-chain antibody, in order to screening antibody-antigen interactions (De Wildt et al. (2000). Nature Biotech. 18, 989-994). These art-known methods and other can be used to generate an array of antibodies for detecting the abundance of polypeptides in a sample. The sample can be labeled, e.g., biotinylated, for subsequent detection with streptavidin coupled to a fluorescent label. The array can then be scanned to measure binding at each address.

Peptide Mimetics

The invention also provides for reduction of the protein binding domains of the subject polypeptides, e.g., LRb or STAT3 or ERK, to generate mimetics, e.g. peptide or non-peptide agents. See, for example, “Peptide inhibitors of human papillomavirus protein binding to retinoblastoma gene protein” European patent applications EP 0 412 762 and EP 0 031 080.

Non-hydrolyzable peptide analogs of critical residues can be generated using benzodiazepine (e.g., see Freidinger et al. in Peptides: Chemistry and Biology, G. R. Marshall ed., ESCOM Publisher: Leiden, Netherlands, 1988), azepine (e.g., see Huffman et al. in Peptides: Chemistry and Biology, G. R. Marshall ed., ESCOM Publisher: Leiden, Netherlands, 1988), substituted gama lactam rings (Garvey et al. in Peptides: Chemistry and Biology, G. R. Marshall ed., ESCOM Publisher: Leiden, Netherlands, 1988), keto-methylene pseudopeptides (Ewenson et al. (1986) J Med Chem 29:295; and Ewenson et al. in Peptides: Structure and Function (Proceedings of the 9th American Peptide Symposium) Pierce Chemical Co. Rockland, Ill., 1985), β-turn dipeptide cores (Nagai et al. (1985) Tetrahedron Lett 26:647; and Sato et al. (1986) J Chem Soc Perkin Trans 1:1231), and β-aminoalcohols (Gordon et al. (1985) Biochem Biophys Res Commun 126:419; and Dann et al. (1986) Biochem Biophys Res Commun 134:71).

Antibodies

The invention also includes antibodies specifically reactive with an LRb-STAT3 signaling molecule, e.g., an LRb or STAT3 described herein, or an LRb-ERK signaling molecule, e.g., an LRb or ERK described herein. An antibody can be an antibody or a fragment thereof, e.g., an antigen binding portion thereof. As used herein, the term “antibody” refers to a protein comprising at least one, and preferably two, heavy (H) chain variable regions (abbreviated herein as VH), and at least one and preferably two light (L) chain variable regions (abbreviated herein as VL). The VH and VL regions can be further subdivided into regions of hypervariability, termed “complementarity determining regions” (“CDR”), interspersed with regions that are more conserved, termed “framework regions” (FR). The extent of the framework region and CDR's has been precisely defined (see, Kabat, E. A., et al. (1991) Sequences of Proteins of Immunological Interest, Fifth Edition, U.S. Department of Health and Human Services, NIH Publication No. 91-3242, and Chothia, C. et al. (1987) J. Mol. Biol. 196:901-917, which are incorporated herein by reference). Each VH and VL is composed of three CDR's and four FRs, arranged from amino-terminus to carboxy-terminus in the following order: FR1, CDR1, FR2, CDR2, FR3, CDR3, FR4.

The antibody can further include a heavy and light chain constant region, to thereby form a heavy and light immunoglobulin chain, respectively. In one embodiment, the antibody is a tetramer of two heavy immunoglobulin chains and two light immunoglobulin chains, wherein the heavy and light immunoglobulin chains are inter-connected by, e.g., disulfide bonds. The heavy chain constant region is comprised of three domains, CH1, CH2 and CH3. The light chain constant region is comprised of one domain, CL. The variable region of the heavy and light chains contains a binding domain that interacts with an antigen. The constant regions of the antibodies typically mediate the binding of the antibody to host tissues or factors, including various cells of the immune system (e.g., effector cells) and the first component (Clq) of the classical complement system.

The term “antigen-binding fragment” of an antibody (or simply “antibody portion,” or “fragment”), as used herein, refers to one or more fragments of a full-length antibody that retain the ability to specifically bind to an antigen (e.g., a polypeptide encoded by a nucleic acid of Group I or II). Examples of binding fragments encompassed within the term “antigen-binding fragment” of an antibody include (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Furthermore, although the two domains of the Fv fragment, VL and VH, are coded for by separate nucleic acids, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding fragment” of an antibody. These antibody fragments are obtained using conventional techniques known to those with skill in the art, and the fragments are screened for utility in the same manner as are intact antibodies. The term “monoclonal antibody” or “monoclonal antibody composition”, as used herein, refers to a population of antibody molecules that contain only one species of an antigen binding site capable of immunoreacting with a particular epitope. A monoclonal antibody composition thus typically displays a single binding affinity for a particular protein with which it immunoreacts.

Anti-protein/anti-peptide antisera or monoclonal antibodies can be made as described herein by using standard protocols (See, for example, Antibodies: A Laboratory Manual ed. by Harlow and Lane (Cold Spring Harbor Press: 1988)).

LRb-STAT3 signaling molecules, e.g., a LRb or STAT3 or a portion or fragment thereof, or LRb-ERK signaling molecules, e.g., LRb or ERK or portions or fragments thereof, can be used as an immunogen to generate antibodies that bind the component using standard techniques for polyclonal and monoclonal antibody preparation. The full-length component protein can be used or, alternatively, antigenic peptide fragments of the component can be used as immunogens.

Typically, a peptide is used to prepare antibodies by immunizing a suitable subject, (e.g., rabbit, goat, mouse or other mammal) with the immunogen. An appropriate immunogenic preparation can contain, for example, a recombinant LRb-STAT3 signaling molecule, e.g., a LRb or STAT3, peptide, or a chemically synthesized LRb-STAT3 signaling molecule, e.g., a LRb or STAT3, peptide or anagonist. See, e.g., U.S. Pat. No. 5,460,959; and co-pending U.S. applications U.S. Ser. No. 08/334,797; U.S. Ser. No. 08/231,439; U.S. Ser. No. 08/334,455; and U.S. Ser. No. 08/928,881 which are hereby expressly incorporated by reference in their entirety. The nucleotide and amino acid sequences of the LRb-STAT3 signaling molecules and the LRb-ERK signaling molecules described herein are known. The preparation can further include an adjuvant, such as Freund's complete or incomplete adjuvant, or similar immunostimulatory agent. Immunization of a suitable subject with an immunogenic LRb-STAT3 signaling molecule (or LRb-ERK signaling molecule) preparation induces a polyclonal anti-LRb-STAT3 signaling molecule antibody response.

Additionally, antibodies produced by genetic engineering methods, such as chimeric and humanized monoclonal antibodies, comprising both human and non-human portions, which can be made using standard recombinant DNA techniques, can be used. Such chimeric and humanized monoclonal antibodies can be produced by genetic engineering using standard DNA techniques known in the art, for example using methods described in Robinson et al. International Application No. PCT/US86/02269; Akira, et al. European Patent Application 184,187; Taniguchi, M., European Patent Application 171,496; Morrison et al. European Patent Application 173,494; Neuberger et al. PCT International Publication No. WO 86/01533; Cabilly et al. U.S. Pat. No. 4,816,567; Cabilly et al. European Patent Application 125,023; Better et al., Science 240:1041-1043, 1988; Liu et al., PNAS 84:3439-3443, 1987; Liu et al., J. Immunol. 139:3521-3526, 1987; Sun et al. PNAS 84:214-218, 1987; Nishimura et al., Canc. Res. 47:999-1005, 1987; Wood et al., Nature 314:446-449, 1985; and Shaw et al., J. Natl. Cancer Inst. 80:1553-1559, 1988); Morrison, S. L., Science 229:1202-1207, 1985; Oi et al., BioTechniques 4:214, 1986; Winter U.S. Pat. No. 5,225,539; Jones et al., Nature 321:552-525, 1986; Verhoeyan et al., Science 239:1534, 1988; and Beidler et al., J. Immunol. 141:4053-4060, 1988.

In addition, a human monoclonal antibody directed against a LRb-STAT3 signaling molecule, e.g., na LRb or STAT3 described herein, or an LRb-ERK signaling molecule, e.g., an LRb or ERK described herein, can be made using standard techniques. For example, human monoclonal antibodies can be generated in transgenic mice or in immune deficient mice engrafted with antibody-producing human cells. Methods of generating such mice are describe, for example, in Wood et al. PCT publication WO 91/00906, Kucherlapati et al. PCT publication WO 91/10741; Lonberg et al. PCT publication WO 92/03918; Kay et al. PCT publication WO 92/03917; Kay et al. PCT publication WO 93/12227; Kay et al. PCT publication 94/25585; Rajewsky et al. Pct publication WO 94/04667; Ditullio et al. PCT publication WO 95/17085; Lonberg, N. et al. (1994) Nature 368:856-859; Green, L. L. et al. (1994) Nature Genet. 7:13-21; Morrison, S. L. et al. (1994) Proc. Natl. Acad. Sci. USA 81:6851-6855; Bruggeman et al. (1993) Year Immunol 7:33-40; Choi et al. (1993) Nature Genet. 4:117-123; Tuaillon et al. (1993) PNAS 90:3720-3724; Bruggeman et al. (1991) Eur J Immunol 21:1323-1326); Duchosal et al. PCT publication WO 93/05796; U.S. Pat. No. 5,411,749; McCune et al. (1988) Science 241:1632-1639), Kamel-Reid et al. (1988) Science 242:1706; Spanopoulou (1994) Genes & Development 8:1030-1042; Shinkai et al. (1992) Cell 68:855-868). A human antibody-transgenic mouse or an immune deficient mouse engrafted with human antibody-producing cells or tissue can be immunized with a LRb-STAT3 signaling molecule, e.g., a LRb or STAT3, described herein or an antigenic peptide thereof, or an LRb-ERK signaling molecule, e.g., an LRb or ERK described herein or an antigenic peptide thereof, and splenocytes from these immunized mice can then be used to create hybridomas. Methods of hybridoma production are well known.

Human monoclonal antibodies against a LRb-STAT3 signaling molecule, e.g., a LRb or STAT3 described herein, or an LRb-ERK signaling molecule, e.g., an LRb or ERK described herein, can also be prepared by constructing a combinatorial immunoglobulin library, such as a Fab phage display library or a scFv phage display library, using immunoglobulin light chain and heavy chain cDNAs prepared from mRNA derived from lymphocytes of a subject. See, e.g., McCafferty et al. PCT publication WO 92/01047; Marks et al. (1991) J. Mol. Biol. 222:581-597; and Griffths et al. (1993) EMBO J 12:725-734. In addition, a combinatorial library of antibody variable regions can be generated by mutating a known human antibody. For example, a variable region of a human antibody known to bind a LRb-STAT3 signaling molecule can be mutated, by for example using randomly altered mutagenized oligonucleotides, to generate a library of mutated variable regions which can then be screened to bind to a LRb-STAT3 signaling molecule, e.g., a LRb or STAT3. Methods of inducing random mutagenesis within the CDR regions of immunoglobin heavy and/or light chains, methods of crossing randomized heavy and light chains to form pairings and screening methods can be found in, for example, Barbas et al. PCT publication WO 96/07754; Barbas et al. (1992) Proc. Nat'l Acad. Sci. USA 89:4457-4461.

The immunoglobulin library can be expressed by a population of display packages, preferably derived from filamentous phage, to form an antibody display library. Examples of methods and reagents particularly amenable for use in generating antibody display library can be found in, for example, Ladner et al. U.S. Pat. No. 5,223,409; Kang et al. PCT publication WO 92/18619; Dower et al. PCT publication WO 91/17271; Winter et al. PCT publication WO 92/20791; Markland et al. PCT publication WO 92/15679; Breitling et al. PCT publication WO 93/01288; McCafferty et al. PCT publication WO 92/01047; Garrard et al. PCT publication WO 92/09690; Ladner et al. PCT publication WO 90/02809; Fuchs et al. (1991) Bio/Technology 9:1370-1372; Hay et al. (1992) Hum Antibod Hybridomas 3:81-85; Huse et al. (1989) Science 246:1275-1281; Griffths et al. (1993) supra; Hawkins et al. (1992) J Mol Biol 226:889-896; Clackson et al. (1991) Nature 352:624-628; Gram et al. (1992) PNAS 89:3576-3580; Garrad et al. (1991)Bio/Technology 9:1373-1377; Hoogenboom et al. (1991) Nuc Acid Res 19:4133-4137; and Barbas et al. (1991) PNAS 88:7978-7982. Once displayed on the surface of a display package (e.g., filamentous phage), the antibody library is screened to identify and isolate packages that express an antibody that binds a LRb-STAT3 signaling molecule, e.g., a LRb or STAT3, described herein. In a preferred embodiment, the primary screening of the library involves panning with an immobilized LRb-STAT3 signaling molecule, e.g., a LRb or STAT3 described herein and display packages expressing antibodies that bind immobilized proteins described herein are selected.

Although many of the methods described herein are discussed with regards to LRb-STAT3 signaling molecules, they are also applicable to LRb-ERK signaling molecules.

Antisense Nucleic Acid Sequences

Nucleic acid molecules which are antisense to a nucleotide encoding a LRb-STAT3 signaling molecule described herein, e.g., a LRb or STAT3, or an LRb-ERK signaling molecule, e.g., an LRb or ERK described herein, can be used as an agent which inhibits expression of the LRb-STAT3 signaling molecule or an LRb-ERK signaling molecule, respectively. An “antisense” nucleic acid includes a nucleotide sequence which is complementary to a “sense” nucleic acid encoding the component, e.g., complementary to the coding strand of a double-stranded cDNA molecule or complementary to an mRNA sequence. Accordingly, an antisense nucleic acid can form hydrogen bonds with a sense nucleic acid. The antisense nucleic acid can be complementary to an entire coding strand, or to only a portion thereof. For example, an antisense nucleic acid molecule which antisense to the “coding region” of the coding strand of a nucleotide sequence encoding the component can be used.

The coding strand sequences encoding LRb-STAT3 signaling molecules or the LRb-ERK signaling molecules described herein are known. Given the coding strand sequences encoding these proteins, antisense nucleic acids can be designed according to the rules of Watson and Crick base pairing. The antisense nucleic acid molecule can be complementary to the entire coding region of mRNA, but more preferably is an oligonucleotide which is antisense to only a portion of the coding or noncoding region of mRNA. For example, the antisense oligonucleotide can be complementary to the region surrounding the translation start site of the mRNA. An antisense oligonucleotide can be, for example, about 5, 10, 15, 20, 25, 30, 35, 40, 45 or 50 nucleotides in length. An antisense nucleic acid can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest.

Administration

An agent which modulates the level of expression of a LRb-STAT3 signaling molecule or an LRb-ERK signaling molecule described herein can be administered to a subject by standard methods. For example, the agent can be administered by any of a number of different routes including intravenous, intradermal, subcutaneous, oral (e.g., inhalation), transdermal (topical), and transmucosal. In one embodiment, the modulating agent can be administered orally. In another embodiment, the agent is administered by injection, e.g., intramuscularly, or intravenously.

The agent which modulates protein levels, e.g., nucleic acid molecules, polypeptides, fragments or analogs, modulators, and antibodies (also referred to herein as “active compounds”) can be incorporated into pharmaceutical compositions suitable for administration to a subject, e.g., a human. Such compositions typically include the nucleic acid molecule, polypeptide, modulator, or antibody and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances arc known. Except insofar as any conventional media or agent is incompatible with the active compound, such media can be used in the compositions of the invention. Supplementary active compounds can also be incorporated into the compositions.

A pharmaceutical composition can be formulated to be compatible with its intended route of administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a LRb-STAT3 signaling molecule, e.g., a LRb or STAT3 polypeptide or antibody) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

Systemic administration can also be by transmucosal or transdermal means. For transmucosal or transdermal administration, penetrants appropriate to the barrier to be permeated are used in the formulation. Such penetrants are generally known, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

The nucleic acid molecules described herein can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al., PNAS 91:3054-3057, 1994). The pharmaceutical preparation of the gene therapy vector can include the gene therapy vector in an acceptable diluent, or can include a slow release matrix in which the gene delivery vehicle is imbedded. Alternatively, where the complete gene delivery vector can be produced intact from recombinant cells, e.g. retroviral vectors, the pharmaceutical preparation can include one or more cells which produce the gene delivery system.

The pharmaceutical compositions can be included in a container, pack, or dispenser together with instructions for administration.

Gene Therapy

The nucleic acids described herein, e.g., a nucleic acid encoding a LRb-STAT3 signaling molecule, e.g., a LRb or STAT3 described herein, or an antisense nucleic acid, can be incorporated into gene constructs to be used as a part of a gene therapy protocol to deliver nucleic acids encoding either an agonistic or antagonistic form of a LRb-STAT3 signaling molecule described herein, e.g., a LRb or STAT3, or LRb-ERK signaling molecules described hereom. The invention features expression vectors for in vivo transfection and expression of a LRb-STAT3 signaling molecule or LRb-ERK signaling molecule described herein in particular cell types so as to reconstitute the function of, or alternatively, antagonize the function of the component in a cell in which that polypeptide is misexpressed. Expression constructs of such components may be administered in any biologically effective carrier, e.g. any formulation or composition capable of effectively delivering the component gene to cells in vivo. Approaches include insertion of the subject gene in viral vectors including recombinant retroviruses, adenovirus, adeno-associated virus, and herpes simplex virus-1, or recombinant bacterial or eukaryotic plasmids. Viral vectors transfect cells directly; plasmid DNA can be delivered with the help of, for example, cationic liposomes (lipofectin) or derivatized (e.g. antibody conjugated), polylysine conjugates, gramacidin S, artificial viral envelopes or other such intracellular carriers, as well as direct injection of the gene construct or CaPO4 precipitation carried out in vivo.

A preferred approach for in vivo introduction of nucleic acid into a cell is by use of a viral vector containing nucleic acid, e.g. a cDNA, encoding a LRb-STAT3 or LRb-ERK signaling molecule, e.g., a LRb or STAT3 or ERK, described herein. Infection of cells with a viral vector has the advantage that a large proportion of the targeted cells can receive the nucleic acid. Additionally, molecules encoded within the viral vector, e.g., by a cDNA contained in the viral vector, are expressed efficiently in cells which have taken up viral vector nucleic acid.

Retrovirus vectors and adeno-associated virus vectors can be used as a recombinant gene delivery system for the transfer of exogenous genes in vivo, particularly into humans. These vectors provide efficient delivery of genes into cells, and the transferred nucleic acids are stably integrated into the chromosomal DNA of the host. The development of specialized cell lines (termed “packaging cells”) which produce only replication-defective retroviruses has increased the utility of retroviruses for gene therapy, and defective retroviruses are characterized for use in gene transfer for gene therapy purposes (for a review see Miller, A. D. (1990) Blood 76:271). A replication defective retrovirus can be packaged into virions which can be used to infect a target cell through the use of a helper virus by standard techniques. Protocols for producing recombinant retroviruses and for infecting cells in vitro or in vivo with such viruses can be found in Current Protocols in Molecular Biology, Ausubel, F. M. et al. (eds.) Greene Publishing Associates, (1989), Sections 9.10-9.14 and other standard laboratory manuals. Examples of suitable retroviruses include pLJ, pZIP, pWE and pEM which are known to those skilled in the art. Examples of suitable packaging virus lines for preparing both ecotropic and amphotropic retroviral systems include *Crip, *Cre, *2 and *Am. Retroviruses have been used to introduce a variety of genes into many different cell types, including epithelial cells, in vitro and/or in vivo (see for example Eglitis, et al. (1985) Science 230:1395-1398; Danos and Mulligan (1988) Proc. Natl. Acad. Sci. USA 85:6460-6464; Wilson et al. (1988) Proc. Natl. Acad. Sci. USA 85:3014-3018; Armentano et al. (1990) Proc. Natl. Acad. Sci. USA 87:6141-6145; Huber et al. (1991) Proc. Natl. Acad. Sci. USA 88:8039-8043; Ferry et al. (1991) Proc. Natl. Acad. Sci. USA 88:8377-8381; Chowdhury et al. (1991) Science 254:1802-1805; van Beusechem et al. (1992) Proc. Natl. Acad. Sci. USA 89:7640-7644; Kay et al. (1992) Human Gene Therapy 3:641-647; Dai et al. (1992) Proc. Natl. Acad. Sci. USA 89:10892-10895; Hwu et al. (1993) J. Immunol. 150:4104-4115; U.S. Pat. No. 4,868,116; U.S. Pat. No. 4,980,286; PCT Application WO 89/07136; PCT Application WO 89/02468; PCT Application WO 89/05345; and PCT Application WO 92/07573).

Another viral gene delivery system useful in the present invention utilizes adenovirus-derived vectors. The genome of an adenovirus can be manipulated such that it encodes and expresses a gene product of interest but is inactivated in terms of its ability to replicate in a normal lytic viral life cycle. See, for example, Berkner et al. (1988) BioTechniques 6:616; Rosenfeld et al. (1991) Science 252:431-434; and Rosenfeld et al. (1992) Cell 68:143-155. Suitable adenoviral vectors derived from the adenovirus strain Ad type 5 dl324 or other strains of adenovirus (e.g., Ad2, Ad3, Ad7 etc.) are known to those skilled in the art. Recombinant adenoviruses can be advantageous in certain circumstances in that they are not capable of infecting nondividing cells and can be used to infect a wide variety of cell types, including epithelial cells (Rosenfeld et al. (1992) cited supra). Furthermore, the virus particle is relatively stable and amenable to purification and concentration, and as above, can be modified so as to affect the spectrum of infectivity. Additionally, introduced adenoviral DNA (and foreign DNA contained therein) is not integrated into the genome of a host cell but remains episomal, thereby avoiding potential problems that can occur as a result of insertional mutagenesis in situ where introduced DNA becomes integrated into the host genome (e.g., retroviral DNA). Moreover, the carrying capacity of the adenoviral genome for foreign DNA is large (up to 8 kilobases) relative to other gene delivery vectors (Berkner et al. cited supra; Haj-Ahmand and Graham (1986) J. Virol. 57:267).

Yet another viral vector system useful for delivery of the subject gene is the adeno-associated virus (AAV). Adeno-associated virus is a naturally occurring defective virus that requires another virus, such as an adenovirus or a herpes virus, as a helper virus for efficient replication and a productive life cycle. (For a review see Muzyczka et al. (1992) Curr. Topics in Micro. and Immunol. 158:97-129). It is also one of the few viruses that may integrate its DNA into non-dividing cells, and exhibits a high frequency of stable integration (see for example Flotte et al. (1992) Am. J. Respir. Cell. Mol. Biol. 7:349-356; Samulski et al. (1989) J. Virol. 63:3822-3828; and McLaughlin et al. (1989) J. Virol. 62:1963-1973). Vectors containing as little as 300 base pairs of AAV can be packaged and can integrate. Space for exogenous DNA is limited to about 4.5 kb. An AAV vector such as that described in Tratschin et al. (1985) Mol. Cell. Biol. 5:3251-3260 can be used to introduce DNA into cells. A variety of nucleic acids have been introduced into different cell types using AAV vectors (see for example Hermonat et al. (1984) Proc. Natl. Acad. Sci. USA 81:6466-6470; Tratschin et al. (1985) Mol. Cell. Biol. 4:2072-2081; Wondisford et al. (1988) Mol. Endocrinol. 2:32-39; Tratschin et al. (1984) J. Virol. 51:611-619; and Flotte et al. (1993) J. Biol. Chem. 268:3781-3790).

In addition to viral transfer methods, such as those illustrated above, non-viral methods can also be employed to cause expression of a LRb-STAT3 or LRb-ERK signaling molecule described herein, e.g., a LRb or STAT3 or ERK, in the tissue of a subject. Most nonviral methods of gene transfer rely on normal mechanisms used by mammalian cells for the uptake and intracellular transport of macromolecules. In preferred embodiments, non-viral gene delivery systems of the present invention rely on endocytic pathways for the uptake of the subject gene by the targeted cell. Exemplary gene delivery systems of this type include liposomal derived systems, poly-lysine conjugates, and artificial viral envelopes. Other embodiments include plasmid injection systems such as are described in Meuli et al. (2001) J Invest Dermatol. 116(1):131-135; Cohen et al. (2000) Gene Ther 7(22):1896-905; or Tam et al. (2000) Gene Ther 7(21):1867-74.

In a representative embodiment, a gene encoding a LRb-STAT3 or LRb-ERK signaling molecule described herein (e.g., LRb or STAT3 or ERK) can be entrapped in liposomes bearing positive charges on their surface (e.g., lipofectins) and (optionally) which are tagged with antibodies against cell surface antigens of the target tissue (Mizuno et al. (1992) No Shinkei Geka 20:547-551; PCT publication WO91/06309; Japanese patent application 1047381; and European patent publication EP-A-43075).

In clinical settings, the gene delivery systems for the therapeutic gene can be introduced into a patient by any of a number of methods, each of which is familiar in the art. For instance, a pharmaceutical preparation of the gene delivery system can be introduced systemically, e.g. by intravenous injection, and specific transduction of the protein in the target cells occurs predominantly from specificity of transfection provided by the gene delivery vehicle, cell-type or tissue-type expression due to the transcriptional regulatory sequences controlling expression of the receptor gene, or a combination thereof. In other embodiments, initial delivery of the recombinant gene is more limited with introduction into the animal being quite localized. For example, the gene delivery vehicle can be introduced by catheter (see U.S. Pat. No. 5,328,470) or by stereotactic injection (e.g. Chen et al. (1994) PNAS 91: 3054-3057).

The pharmaceutical preparation of the gene therapy construct can consist essentially of the gene delivery system in an acceptable diluent, or can comprise a slow release matrix in which the gene delivery vehicle is imbedded. Alternatively, where the complete gene delivery system can be produced in tact from recombinant cells, e.g. retroviral vectors, the pharmaceutical preparation can comprise one or more cells which produce the gene delivery system.

Cell Therapy

A LRb-STAT3 or LRb-ERK signaling molecule described herein, e.g., a LRb or STAT3 or ERK, can also be increased in a subject by introducing into a cell, e.g., an endothelial cell, a nucleotide sequence that modulates the production of an LRb-STAT3 or an LRb-ERK signaling molecule, e.g., a LRb or STAT3 or ERK, e.g., a nucleotide sequence encoding a LRb-STAT3 or LRb-ERK signaling molecule, e.g., a LRb or STAT3 or ERK, polypeptide or functional fragment or analog thereof, a promoter sequence, e.g., a promoter sequence from a LRb-STAT3 or LRb-ERK signaling molecule, e.g., a LRb or STAT3 or ERK, gene or from another gene; an enhancer sequence, e.g., 5′ untranslated region (UTR), e.g., a 5′ UTR from a LRb-STAT3 or LRb-ERK signaling molecule, e.g., a LRb or STAT3 or ERK, gene or from another gene, a 3′ UTR, e.g., a 3′ UTR from a LRb-STAT3 or LRb-ERK signaling molecule gene or from another gene; a polyadenylation site; an insulator sequence; or another sequence that modulates the expression of the LRb-STAT3 or LRb-ERK signaling molecule, e.g., a LRb or STAT3 or ERK. The cell can then be introduced into the subject.

Primary and secondary cells to be genetically engineered can be obtained form a variety of tissues and include cell types which can be maintained propagated in culture. For example, primary and secondary cells include fibroblasts, keratinocytes, epithelial cells (e.g., mammary epithelial cells, intestinal epithelial cells), endothelial cells, glial cells, neural cells, formed elements of the blood (e.g., lymphocytes, bone marrow cells), muscle cells (myoblasts) and precursors of these somatic cell types. Primary cells are preferably obtained from the individual to whom the genetically engineered primary or secondary cells are administered. However, primary cells may be obtained for a donor (other than the recipient).

The term “primary cell” includes cells present in a suspension of cells isolated from a vertebrate tissue source (prior to their being plated i.e., attached to a tissue culture substrate such as a dish or flask), cells present in an explant derived from tissue, both of the previous types of cells plated for the first time, and cell suspensions derived from these plated cells. The term “secondary cell” or “cell strain” refers to cells at all subsequent steps in culturing. Secondary cells are cell strains which consist of secondary cells which have been passaged one or more times.

Primary or secondary cells of vertebrate, particularly mammalian, origin can be transfected with an exogenous nucleic acid sequence which includes a nucleic acid sequence encoding a signal peptide, and/or a heterologous nucleic acid sequence, e.g., encoding a LRb-STAT3 or LRb-ERK signaling molecule, e.g., a LRb or STAT3 or ERK, or an agonist or antagonist thereof, and produce the encoded product stably and reproducibly in vitro and in vivo, over extended periods of time. A heterologous amino acid can also be a regulatory sequence, e.g., a promoter, which causes expression, e.g., inducible expression or upregulation, of an endogenous sequence. An exogenous nucleic acid sequence can be introduced into a primary or secondary cell by homologous recombination as described, for example, in U.S. Pat. No. 5,641,670, the contents of which are incorporated herein by reference. The transfected primary or secondary cells may also include DNA encoding a selectable marker which confers a selectable phenotype upon them, facilitating their identification and isolation.

Vertebrate tissue can be obtained by standard methods such a punch biopsy or other surgical methods of obtaining a tissue source of the primary cell type of interest. For example, punch biopsy is used to obtain skin as a source of fibroblasts or keratinocytes. A mixture of primary cells is obtained from the tissue, using known methods, such as enzymatic digestion or explanting. If enzymatic digestion is used, enzymes such as collagenase, hyaluronidase, dispase, pronase, trypsin, elastase and chymotrypsin can bc used.

The resulting primary cell mixture can be transfected directly or it can be cultured first, removed from the culture plate and resuspended before transfection is carried out. Primary cells or secondary cells are combined with exogenous nucleic acid sequence to, e.g., stably integrate into their genomes, and treated in order to accomplish transfection. As used herein, the term “transfection” includes a variety of techniques for introducing an exogenous nucleic acid into a cell including calcium phosphate or calcium chloride precipitation, microinjection, DEAE-dextrin-mediated transfection, lipofection or electrophoration, all of which are routine in the art.

Transfected primary or secondary cells undergo sufficient number doubling to produce either a clonal cell strain or a heterogeneous cell strain of sufficient size to provide the therapeutic protein to an individual in effective amounts. The number of required cells in a transfected clonal heterogeneous cell strain is variable and depends on a variety of factors, including but not limited to, the use of the transfected cells, the functional level of the exogenous DNA in the transfected cells, the site of implantation of the transfected cells (for example, the number of cells that can be used is limited by the anatomical site of implantation), and the age, surface area, and clinical condition of the patient.

The transfected cells, e.g., cells produced as described herein, can be introduced into an individual to whom the product is to be delivered. Various routes of administration and various sites (e.g., renal sub capsular, subcutaneous, central nervous system (including intrathecal), intravascular, intrahepatic, intrasplanchnic, intraperitoneal (including intraomental), intramuscularly implantation) can be used. One implanted in individual, the transfected cells produce the product encoded by the heterologous DNA or are affected by the heterologous DNA itself. For example, an individual who suffers from an insulin related disorder is a candidate for implantation of cells producing an antagonist of LRb-STAT3 signaling molecule described herein.

An immunosuppressive agent e.g., drug, or antibody, can be administered to a subject at a dosage sufficient to achieve the desired therapeutic effect (e.g., inhibition of rejection of the cells). Dosage ranges for immunosuppressive drugs are known in the art. See, e.g., Freed et al. (1992) N. Engl. J. Med. 327:1549; Spencer et al. (1992) N. Engl. J. Med. 327:1541′ Widner et al. (1992) n. Engl. J. Med. 327:1556). Dosage values may vary according to factors such as the disease state, age, sex, and weight of the individual.

### EXAMPLES

### Example 1

### Transgenic Mice

In one embodiment, the inventors created a transgenic mouse in which the gene for the leptin receptor (lepr) was replaced in murine R1 ES cells with an allele containing a substitution mutant of Tyr1138 (leprS1138) by homologous targeting to generate “knock-in” mice that express LRbS1138 in a manner identical to wild-type LRb (FIG. 1A). 129Sv ES cell clones that contained a correctly targeted Neo cassette and the leprS1138 mutation (which creates a novel Sph I site) were injected into C57BL/6J blastocyts to produce chimeric animals, which were crossed with C57BL/6J mice to generate heterozygous animals on a mixed C57BL/6Jx129Sv background. Interbreeding of heterozygous animals yielded wild type (+/+), heterozygous (s/+), and homozygous (s/s) mice for study at Mendelian frequencies (FIG. 1B).

### Example 2

### Animals

C57BL/6J db/+ and db/db animals were obtained from Jackson Laboratories (Bar Harbor, Me.) at four weeks of age. C57BL/6J db/+ breeder pairs were also obtained from Jackson labs to generate additional mice for study. All mice were housed in the Joslin Diabetes Center's accredited mouse barrier facility with ad libidum access to standard chow and water, except those mice that were pair-fed to wild-type mice, which were given ad libidum access to water and fed the amount of chow eaten the previous day by a wild-type littermate. Except for mice in breeding studies, which were housed in pairs, all mice were housed singly from the time of weaning at 21 days.

### Example 3

### In situ Hybridization

Brains were removed immediately after decapitation, frozen in a bed of crushed dry ice, sectioned in a coronal plane at 14 μm with a cryostat, mounted on RNase-free slides, and treated with 4% paraformaldehyde, acetic anhydride, ethanol and chloroform²⁸. For each animal, 6 slides (12 brain sections) containing hypothalamus were selected from the midregion of the rostro-caudal extent of the arcuate nucleus (which also contains the ventromedial nuclei). The anatomical equivalence of hypothalamic sections among animals was obtained by selecting slides (viewed with a darkfield stereomicroscope) with the aid of a rat brain atlas prior to hybridization. All brain slices were concurrently prepared for hybridization and used in the same assay. Hybridization to LRb mRNA was performed with an antisense riboprobe transcribed from a cDNA template using (³³P) UTP²⁹, and unincorporated label was separated using a QIAquick Nucleotide Removal Kit (Qiagen). The hybridization signal was detected using a phosphoimager system (Packard).

### Example 4

### Semi-Quantitative RT-PCR

Hypothalami were isolated from ad libidum-fed mice in the eighth week of age between 1000 and 1200 hours and snap-frozen. Total hypothalamic RNA was isolated using Ultraspec reagent (Biotecx), and subjected to automated fluorescent RT-PCR on an ABI 7700 for determination of relative RNA concentration. Primers used were: ctgcttcagacctccatagatgtg (forward POMC), cagcgagaggtcgagtttgc (reverse POMC), 6FAM-caacctgctggcttgcatccgg-TAMRA (probe POMC); tcagacctcttaatgaaggaaagca (forward NPY), gagaacaagtttcatttcccatca (reverse NPY), 6FAM-ccagaacaaggcttgaagacccttccat-TAMRA (probe NPY); cagaagctttggcggaggt (forward AgRP), aggactcgtgcagccttacac (reverse AgRP), 6FAM-ctagatccacagaaccgcgagtctcgtt-TAMRA (probe AgRP). Each predicted RT-PCR product spanned an intron/exon junction. Each RT-PCR reaction was determined to be in the linear range for quantitation by comparison to serial dilutions of input standard hypothalamic RNA.

### REFERENCES

1. Chua, S. C., Jr. et al. Phenotypes of mouse diabetes and rat fatty due to mutations in the OB (Leptin) receptor. Science 271, 994-996 (1996).

2. Lee, G. H. et al. Abnormal splicing of the leptin receptor in diabetic mice. Nature 379, 632-635 (1996).

3. Chen, H. et al. Evidence that the diabetes gene encodes the leptin receptor: Identification of a mutation in the leptin receptor gene in db/db mice. Cell 84, 491-495 (1996).

4. Tartaglia, L. A. et al. Identification and expression cloning of a leptin receptor, OB-R. Cell 83, 1263-1271 (1995).

5. Baumann, H. et al. The full-length leptin receptor has signaling capabilities of interleukin 6-type cytokine receptors. Proc. Natl. Acad. Sci. U.S.A. 93, 8374-8378 (1996).

6. Bjorbaek, C., Uotani, S., da Silva, B. & Flier, J. S. Divergent signaling capacities of the long and short isoforms of the leptin receptor. J. Biol. Chem. 272, 32686-32695 (1997).

7. Banks, A. S., Davis, S. M., Bates, S. H. & Myers, M. G., Jr. Activation of downstream signals by the long form of the leptin receptor. J Biol Chem 275, 14563-14572 (2000).

8. Stahl, N. et al. Choice of STATs and other substrates specified by modular tyrosine-based motifs in cytokine receptors. Science 267, 1349-1353 (1995).

9. White, D. W., Kuropatwinski, K. K., Devos, R., Baumann, H. & Tartaglia, L. A. Leptin receptor (OB-R) signaling. J. Biol. Chem. 272, 4065-4071 (1997).

10. Bjorbaek, C. et al. SOCS3 mediates feedback inhibition of the leptin receptor via Tyr985. J Biol Chem. 2000.

Ref Type: In Press

11. Vaisse, C. et al. Leptin activation of Stat3 in the hypothalamus of wild-type and ob/ob mice but not db/db mice. Nat Genet 14, 95-97 (1996).

12. Bjorbaek, C. et al. Divergent roles of SHP-2 in ERK activation by leptin receptors. J Biol Chem 276, 4747-4755 (2001).

13. Ahima, R. S. et al. Role of leptin in the neuroendocrine response to fasting. Nature 382, 250-252 (1996).

14. Heiman, M. L. et al. Leptin inhibition of the hypothlamic-pituitary-adrenal axis in response to stress. Endocrinology 138, 3859-3863 (1997).

Chehab, F. F., Lim, M. E. & Lu, R. Correction of the sterility defect in homozygous obese female mice by treatment with the human recombinant leptin. Nat Genet 12, 318-320 (1996).

Montague, C. T. et al. Congenital leptin deficiency is associated with severe early onset obesity in humans. Nature 387, 903-908 (1997).

17. Clement, K. et al. A mutation in the human leptin receptor gene causes obesity and pituitary dysfunction. Nature 392, 398-401 (1998).

18. Elias, C. F. et al. Leptin differentially regulates NPY and POMC neurons projecting to the lateral hypothalamic area. Neuron 23, 775-786 (1999).

19. Elmquist, J. K., Maratos-Flier, E., Saper, C. B. & Flier, J. S. Unraveling the central nervous system pathways underlying responses to leptin. Nature Neuroscience. 1, 445-449 (1998).

20. Cowley, M. A. et al. Leptin activates anorexigenic POMC neurons through a neural network in the arcuate nucleus. Nature 411, 480-484 (2001).

21. Spanswick, D., Smith, M. A., Groppi, V. E., Logan, S. D. & Ashford, M. L. Leptin inhibits hypothalamic neurons by activation of ATP-sensitive potassium channels. Nature 390, 521-525 (1997).

22. Ollmann, M. M. et al. Antagonism of central melanocortin receptors in vitro and in vivo by agouti-related protein. Science 278, 135-138 (1997).

23. Erickson, J. C., Hollopeter, G. & Palmiter, R. D Attenuation of the obesity syndrome of ob/ob mice by the loss of neuropeptide Y. Science 274, 1704-1707 (1996).

24. Gruaz, N. M., Pierroz, D. D., Rohner-Jeanrenaud, F., Sizonenko, P. C. & Aubert, M. L. Evidence that neuropeptide Y could represent a neuroendocrine inhibitor of sexual maturation in unfavorable metabolic conditions in the rat. Endocrinology 133, 1891-1894 (1993).

25. Catzeflis, C. et al. Neuropeptide Y administered chronically into the lateral ventricle profoundly inhibits both the gonadotropic and the somatotropic axis in intact adult female rats. Endocrinology 132, 224-234 (1993).

26. Ray, D. W., Ren, S. G. & Melmed, S. Leukemia inhibitory factor (LIF) stimulates proopiomelanocortin (POMC) expression in a corticotroph cell line. Role of STAT pathway. J Clin Invest 97, 1852-1859 (1996).

27. Spanswick, D., Smith, M. A., Mirshamsi, S., Routh, V. H. & Ashford, M. L. Insulin activates ATP-sensitive K+ channels in hypothalamic neurons of lean, but not obese rats. Nat Neurosci 3, 757-758 (2000).

28. Schwartz, M. W. et al. Leptin increases hypothalamic pro-opiomelanocortin mRNA expression in the rostral arcuate nucleus. Diabetes 46, 2119-2123 (1997).

29. Baskin, D. G., Breininger, J. F. & Schwartz, M. W. Leptin receptor mRNA identifies a subpopulation of neuropeptide Y neurons activated by fasting in rat hypothalamus. Diabetes 48, 828-833 (1999).

30. Salmon, D. M. & Flatt, J. P. Effect of dietary fat content on the incidence of obesity among ad libitum fed mice. Int. J Obes. 9, 443-449 (1985).

We claim:

1. A method of modulating appetite or body weight in a mammal, comprising:
identifying a mammal in need of modulating appetite and/or body weight; and
administering to the mammal an agent that modulates leptin receptor long form (LRb)-STAT3 signaling.

2. The method of claim 1, wherein appetite or body weight are modulated in the mammal without an effect on a reproductive function.

3. The method of claim 1, wherein the agent promotes or increases LRb-STAT3 signaling, to thereby decrease appetite and/or body weight.

4. The method of claim 3, wherein the mammal is obese.

5. The method of claim 3, wherein LRb-STAT3 signaling is increased by administering an agent that promotes an LRb-STAT3 signaling activity.

6. The method of claim 5, wherein the agent that promotes LRb-STAT3 signaling activity increases or mimics the phosphorylation of the tyrosine located at about amino acid 1141 of human LRb or the corresponding tyrosine residue in another mammal.

7. The method of claim 5, wherein the agent is a kinase or a kinase agonist.

8. The method of claim 5, wherein the agent is a STAT3 or functional fragment thereof.

9. The method of claim 5, wherein the agent is a small molecule that increases expression of STAT3, POMC or αMSH.

10. The method of claim 5, wherein the agent is a nucleotide sequence encoding STAT3, POMC or αMSH.

11. The method of claim 1, wherein LRb-STAT3 signaling is decreased, to thereby increase appetite or body weight.

12. The method of claim 11, wherein the mammal suffers from a disorder that causes weight loss or decreased appetite.

13. The method of claim 11, wherein the mammal is, has or will be administered a treatment which results in weight loss or decreased appetite.

14. The method of claim 11, wherein LRb-STAT3 signaling is decreased by administering an agent that decreases an LRb-STAT3 signaling activity.

15. The method of claim 14, wherein the agent inhibits phosphorylation of human LRb Tyr¹¹⁴¹ or a corresponding tyrosine in another mammal.

16. The method of claim 15, wherein the agent is a kinase inhibitor or a phosphatase.

17. The method of claim 14, wherein the agent is an anti-STAT3, anti-POMC or anti-αMSH antibody.

18. The method of claim 14, wherein the agent is a STAT3, POMC or αMSH antisense molecule.

19. A non-human transgenic rodent whose genome is heterozygous or homozygous for an engineered mutation in an LRb gene, wherein the mutation affects body weight or appetite in the mammal, but does not substantially affect reproductive function.

20. The transgenic rodent of claim 19, wherein the mutation causes the disruption of LRb Tyr1138 in a mouse or a corresponding tyrosine in another mammal.

21. The transgenic rodent of claim 19, wherein the rodent is a mouse.

22. A cell having a mutation in the LRb gene wherein the mutation causes a disruption in LRb-STAT3 signaling within the cell but does not cause a disruption in LRb-ERK signaling.

23. The cell of claim 22, wherein LRb Tyr985 or a corresponding tyrosine is not mutated.

24. A method of evaluating an agent for the ability to modulate appetite or body weight, the method comprising:
administering a test agent to a transgenic mammal having a mutation in the LRb gene wherein the mutation causes a reduction in LRb-STAT3 signaling but not in LRb-ERK signaling; and
determining whether the test agent affects appetite or body weight in the transgenic mammal.

25. The method of claim 24, wherein the transgenic mammal is evaluated for one or more of: (1) body weight; (2) glucose levels; (3) glucocorticoid levels; (4) leptin levels; (5) whole body triglyceride levels; (6) adiposity; (7) feeding behavior; (8) energy expenditure; (9) rate of weight gain; (10) insulin levels; (11) melanocortin levels; (12) ability to lactate post-partum; (13) fertility; (14) presence of estrous cycles; (15) morphology of reproductive tract; (16) animal length or height.

26. A method of evaluating an agent for the ability to modulate appetite or body weight, the method comprising:
administering a test agent to a cell or tissue having a mutation in the LRb gene wherein the mutation causes a disruption in LRb-STAT3 signaling but not in LRb-ERK signaling; and
determining whether the test agent affects LRb-STAT3 signaling in the cell or tissue.

27. The method of claim 26, wherein determining whether the test agent affects LRb-STAT3 signaling comprises evaluating one or more of the following parameters: 1) phosphorylation state of LRb1138 (or a corresponding tyrosine); 2) Jak3 expression levels or activity; 3) STAT3 expression levels or activity; 4) POMC expression levels or activity; 5) αMSH expression levels or activity; 6) phosphorylation state of LRb985 (or a corresponding tyrosine); 7) ERK expression levels or activity; 8) SHP-2 expression levels or activity; and 9) SOCS3 expression levels or activity.

28. The method of claim 27, further comprising the step of administering the test agent to an experimental animal.

29. The method of claim 24 or 26, wherein the test agent is selected from the group consisting of: a nucleic acid, a polypeptide and a small molecule.

30. A method of determining if a subject is at risk for obesity, the method comprising: (a) evaluating a LRb-STAT3 activity in the subject, and (b) correlating the LRb-STAT3 activity to a risk for obesity, wherein a lower LRb-STAT3 activity in the subject compared to a reference value indicates that the subject is at risk for obesity.

31. A method of determining if a subject is at risk for obesity, the method comprising: (a) determining the presence or absence of a genetic lesion in the LRb gene of a subject, wherein the genetic lesion disrupts the tyrosine located at about amino acid 1141 of LRb, and (b) correlating the presence or absence of the genetic lesion to a risk for obesity, wherein the presence of the genetic lesion is indicative of risk for obesity in the subject.

32. A method of modulating fertility in a mammal, the method comprising modulating LRb-ERK signaling to thereby modulate fertility.

33. A method of modulating height in a mammal, the method comprising modulating LRb-ERK signaling to thereby modulate height.

34. A non-human transgenic rodent whose genome is heterozygous or homozygous for an engineered mutation in an LRb gene, wherein the mutation affects reproductive function or height in the mammal, but does not substantially affect body weight or appetite.

35. A cell having a mutation in the LRb gene wherein the mutation causes a disruption in LRb-ERK signaling within the cell but does not cause a disruption in LRb-STAT3 signaling.

36. The cell of claim 35, wherein LRb Tyr1141 or a corresponding tyrosine is not mutated.

37. A method of evaluating an agent for the ability to modulate reproductive function or height, the method comprising:
administering a test agent to a transgenic mammal having a mutation in the LRb gene wherein the mutation causes a reduction in LRb-ERK signaling but not in LRb-STAT signaling; and
determining whether the test agent affects reproductive function or height in the transgenic mammal.

38. A method of evaluating an agent for the ability to modulate reproductive function or height, the method comprising:
administering a test agent to a cell or tissue having a mutation in the LRb gene wherein the mutation causes a reduction in LRb-ERK signaling but not in LRb-STAT3 signaling; and
determining whether the test agent affects LRb-ERK signaling in the cell or tissue.

39. The method of claim 38, further comprising the step of administering the test agent to an experimental animal.

40. A method of determining if a subject is at risk for a reproductive disorder, the method comprising:
(a) evaluating a LRb-ERK activity in the subject, and (b) correlating the LRb-ERK activity to a risk for reproductive disorder, wherein a lower LRb-ERK activity in the subject compared to a reference value indicates that the subject is at risk for reproductive disorder.

41. A method of determining if a subject is at risk for reproductive disorder, the method comprising:
(a) determining the presence or absence of a genetic lesion in the LRb gene of a subject, wherein the genetic lesion disrupts the tyrosine located at about amino acid 985 of LRb, and
(b) correlating the presence or absence of the genetic lesion to a risk for reproductive disorder, wherein the presence of the genetic lesion is indicative of risk for reproductive disorder in the subject.

42. An isolated LRb polypeptide wherein the tyrosine residue corresponding to murine LRb Tyr 1138 has been altered, but the amino acid residue corresponding to murine LRb Tyr 985 has not been altered.

43. The polypeptide of claim 42, wherein the polypeptide comprises SEQ ID NO:4 wherein the amino acid residue at position 1141 has been altered.

44. The polypeptide of claim 42, wherein the polypeptide comprises SEQ ID NO:2 wherein the amino acid residue at position 1138 has been altered.
